SEQUENCE LISTING (4 sequences)

SEQ ID NO: 1 - DNA, 16 bp, Artificial Sequence
  Primer - P1 AcGFP1.FOR.V4
cagtcgacggtaccgc

SEQ ID NO: 2 - DNA, 18 bp, Artificial Sequence
  Primer - P2 AcGFP1.REV.V4
gccctcgaacttcacctc

SEQ ID NO: 3 - DNA, 18 bp, Artificial Sequence
  P3 AcGFP1.FOR.V2
cgcgggcccgggatccac

SEQ ID NO: 4 - DNA, 19 bp, Artificial Sequence
  P4 AcGFP.REV.V2
acctcggcgcgcgacttgt

(19) United States
(12) Patent Application Publication
(10) Pub. No.: US 2024/0082416 A1
(43) Pub. Date: Mar. 14, 2024

(54) AMATOXIN ANALOGS AND USES THEREOF

(71) Applicants: THE UNIVERSITY OF BRITISH COLUMBIA, Vancouver (CA); HEIDELBERG PHARMA RESEARCH GMBH, Ladenburg (DE)

(72) Inventors: David Perrin, Vancouver (CA); Kaveh Matinkhoo, Vancouver (CA); Francesca Gallo, Ladenburg (DE); Alexandra Braun, Ladenburg (DE); Torsten Hechler, Ladenburg (DE); Christoph Müller, Ladenburg (DE); Andreas Pahl, Ladenburg (DE)

(21) Appl. No.: 18/256,193

(22) PCT Filed: Dec. 7, 2021
(86) PCT No.: PCT/CA2021/051756

§ 371 Date: Jun. 6, 2023

Related U.S. Application Data
(60) Provisional application No. 63/122,655, filed on Dec. 8, 2020.

(51) Int. Cl. A61K 47/68; A61K 47/54; A61P 35/00; C07K 7/64

(52) U.S. Cl. CPC A61K 47/6831; A61K 47/545; A61K 47/6855; A61P 35/00; C07K 7/64

(57) ABSTRACT

This application relates to amatoxin analogs in which the trans-4-substituent on the proline residue of the amatoxin is an R group other than a hydroxy, constructs comprising such amatoxin analogs coupled to a linker and conjugates comprising such amatoxin analog-linker constructs conjugated to a target moiety. The application also relates to uses of such amatoxin analogs, for example, in treatment of cancer. The analog is a compound of Formula I, wherein R1 is not OH:

### CROSS-REFERENCE TO RELATED APPLICATIONS

The present disclosure claims the benefit of priority from co-pending U.S. provisional application No. 63/122,655 filed on Dec. 8, 2020, the contents of which are incorporated herein by reference in their entirety.

### FIELD

The present disclosure relates to amatoxin analogs, constructs comprising such amatoxin analogs coupled to a linker and conjugates comprising such amatoxin analogs. The present disclosure also relates to uses of such amatoxin analogs, for example, in treatment of cancer.

### BACKGROUND

α-Amanitin, the principal toxin found in Amanita phalloides—the notorious “death-cap” mushroom—is one of the deadliest toxins found in Nature (ID₅₀=50-100 μg/kg).^{1,2,3} In addition to its unique bicyclic peptide structure owing to a 6-hydroxytryptathionine-(R)-sulfoxide staple, α-amanitin contains two oxidized unnatural amino acids that have been shown or are thought to be related to its cytotoxicity: trans-4-hydroxyproline (Hyp) and (2S,3R,4R)-4,5-dihydroxyisoleucine (DHIle).^{4,5} Isolated over 70 years ago,¹ α-amanitin is featured in most modem biochemistry textbooks as a fascinating example of a highly selective allosteric inhibitor of eukaryotic RNA polymerase II (Pol II) with a Kᵢ value of 1-10 nM.^{6,7}

Because Pol II is essential for cellular growth and homeostasis, α-amanitin kills dividing and quiescent cells by inhibiting Pol II, which leads to rapid proteolytic degradation of Pol II and finally cell death.⁸ This unique mechanism of action distinguishes α-amanitin from other chemotherapeutics and toxins for targeted therapy that act primarily on rapidly growing cells, therefore α-amanitin may be a useful payload for antibody drug conjugates (ADC). Following early reports on an amanitin-antibody conjugate against Thy 1.2 antigen towards T lymphoma S49.1 cells,⁹ Moldenhauer et al. demonstrated the promise of α-amanitin as a payload for ADC development; an anti-EpCAM antibody-amanitin conjugate cured 60% (3 of 5) mice with pancreatic tumor xenografts,¹⁰ paving the way for HDP-101, an amanitin-based ADC for the treatment of multiple myeloma targeting B cell maturation antigen (BCMA) and the first amanitin ADC advancing towards clinical trials.¹¹ Others have explored the applications of targeted amanitin, underscoring impending interest in this payload and its chemistry.¹² In contrast, direct intraperitoneal injection of α-amanitin was reported to prevent cancer relapse in mice bearing tumor xenografts that are resistant to common chemotherapeutics, suggesting potential for using α-amanitin and analogs as systemic chemotherapeutics without the use of a targeting agent.¹³

Recent X-ray diffraction (XRD) studies on α-amanitin-Pol II co-crystal obtained by Komberg and coworkers revealed multiple interactions between this toxin and the bridge helix of Pol II.¹⁴ Evidenced by this co-crystal structure are the numerous interactions with backbone amides, π-stacking/π-cation interactions with the hydroxytryptathionine, and notable H-bonds to the hydroxyl groups of Hyp and DHIle, the significance of which was further corroborated by recent cryo-EM structures of the same complex obtained by Cramer and coworkers.^{15,16} Notable from the crystal structures is, for example, the H-bonding interactions between the hydroxyl group of Hyp and Glu822 and His1085 of the Rpb1 subunit of Pol II.

These structural studies cohere with structure-activity relationship (SAR) reports dating from the 1980s that were based on derivatives of the natural product along with both naturally occurring and synthetic amatoxins bearing fewer hydroxyl groups. For example, reductive deoxygenation of the (R)-sulfoxide to the thioether or oxidation to the sulfone resulted in no loss of activity in the case of the 6′-O-methyl ether of α-amanitin.¹⁷ Meta-periodate-oxidation of 4,5-dihydroxyisoleucine (DHIle) followed by reduction to hydroxyvaline gave a toxin that displayed an 8-fold higher Kᵢ value.¹⁸ Naturally occurring amatoxins, amanullin and proamanullin, which lack the hydroxyl groups on DHIle or on both DHIle and Hyp, showed Kᵢ values of 10-40 nM (near-native) and 5-20 μM (greatly attenuated) respectively,^{4,18,19}. Synthetic “tetradeoxy-amanitin” (lacking the sulfoxide, the 6-hydroxy group on tryptathionine, and where Ile replaced DHIle) gave a Kᵢ value of 80 nM in an in vitro transcription assay¹⁹ in the same report, yet a gave a much elevated Kᵢ of 1 μM in a different report by the same lab.⁵ The role of DHIle has been further explored using 4-hydroxy-Ile diastereomers: an amanitin analog bearing (2S,3R,4S)-4-hydroxyisoleucine showed no change in Kᵢ in an in vitro Pol II assay, while others gave variable reduction in toxicity of approximately 10-20 fold.²⁰

In amanitin, trans-(4R)-hydroxyproline, which exists in the favored trans-amide bond, adopts the classic Cγ-exo ring pucker that is seen for Hyp in collagen and known to be favored by electron-withdrawing substituents (EWG) that intensify the gauche interaction between the amide bond and the electron-withdrawing group (EWG, Scheme 1).^{21,22,23}

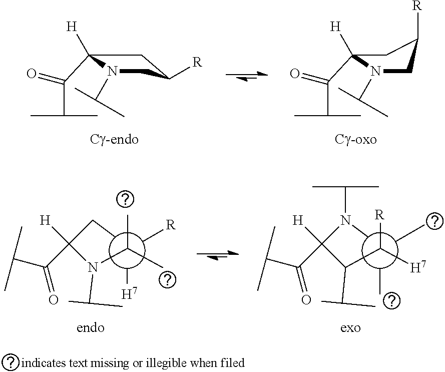

Scheme 1. exo and endo ring puckers observed in the proline residue of a trans-4-substituted proline-containing peptide. (R=EWG) owing to the hyperconjugation of the electron-rich σ(C—H) orbital and the electron-deficient σ(C—R).

In 2018, the first total synthesis of α-amanitin was completed, which overcame several synthetic challenges, most notably an enantioselective synthesis of (2S,3R,4R)-4,5-dihydroxyisoleucine on scale.²⁴ In 2020, two other total syntheses were reported.^{25,26}

### SUMMARY

Herein the synthesis and biochemical evaluation of several derivatives of α-amanitin that contain analogs of its trans-hydroxyproline (Hyp) residue is reported. These α-amanitin analogs were characterized by in vitro transcription assays and cytotoxicity assays. Surprisingly, α-amanitin is exquisitely intolerant of substitution of the hydroxyl group on proline. However, the reduced but still substantial inhibitory activity of some α-amanitin analogs show the potential of these analogs for therapeutic applications, for example, if used as payload for conjugates such as antibody-drug conjugates (ADCs). The combination of being a poor substrate for OATP1B3 transporters whilst retaining inhibitory activity of amanitin to a certain degree might, for example help to develop conjugates with reduced payload-mediated toxicity and/or an improved target-specific effect.

Accordingly, in one aspect the present invention relates to a compound of formula Ama-R¹, wherein Ama is an amatoxin, and R¹ is a trans-4-substituent on the proline residue of the amatoxin, wherein R¹ is not OH.

In an embodiment of the present invention, Ama-R¹ is a compound of Formula I:

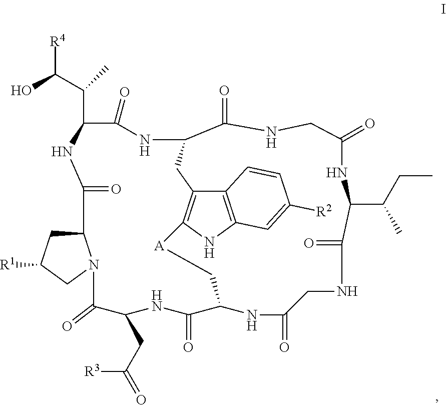

wherein
   R¹ is as defined for Ama-R¹;
   R² is H or OH;
   R³ is NHR⁵ or OR⁶;
   R⁴ is H, CH₃, CH₂OH or CH₂OC(O)R⁷;
   R⁵ is selected from H, NHR^{B}, NHOR⁹, C₁₋₆alkyl and aryl;
   R⁶, R⁷, R⁸ and R⁹ are each independently selected from H, C₁₋₆alkyl and aryl; and
   A is S, (R)—SO or SO₂.

In an embodiment of the present invention, R³ is NH₂.

In an embodiment of the present invention, R⁴ is CH₂OH.

In an embodiment of the present invention, the compound of Formula I is a compound of Formula I(a):

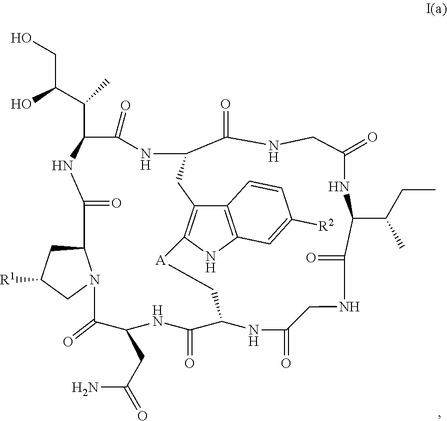

wherein
   R¹ is as defined for Ama-R¹;
   R² is H or OH; and
   A is S, (R)—SO or SO₂.

In an embodiment of the present invention, R² is H. In another embodiment of the present invention, R² is OH.

In an embodiment of the present invention, A is S. In another embodiment of the present invention, A is (R)—SO.

In an embodiment of the present invention, R¹ is —NH₂, —NC(NH₂)², —CN or —SH. In another embodiment of the present invention, R¹ is —CN. In another embodiment of the present invention, R¹ is —NC(NH₂)₂. In another embodiment of the present invention, R¹ is —SH.

In another aspect the present invention relates to a compound-linker construct comprising a compound of the present invention coupled to a linker, wherein the linker comprises a reactive group R¹⁰ for conjugating the compound-linker construct to a target-binding moiety.

In an embodiment of the present invention, the linker is a stable linker. In another embodiment of the present invention, the linker is a cleavable linker. In another embodiment of the present invention, the linker further comprises a self-immolating moiety. In another embodiment of the present invention, the linker is cleavable by at least one agent selected from the group consisting of cysteine protease, metalloproteinase, serine protease, threonine protease and aspartic protease. In another embodiment of the present invention, the linker comprises a motif selected from the group consisting of: Val-Ala, Val-Cit, Val-Lys, Val-Arg, Phe-Lys-Gly-Pro-Leu-Gly, Ala-Ala-Pro-Val, β-glucuronide and β-galactoside.

In an embodiment of the present invention, R¹⁰ is selected from:

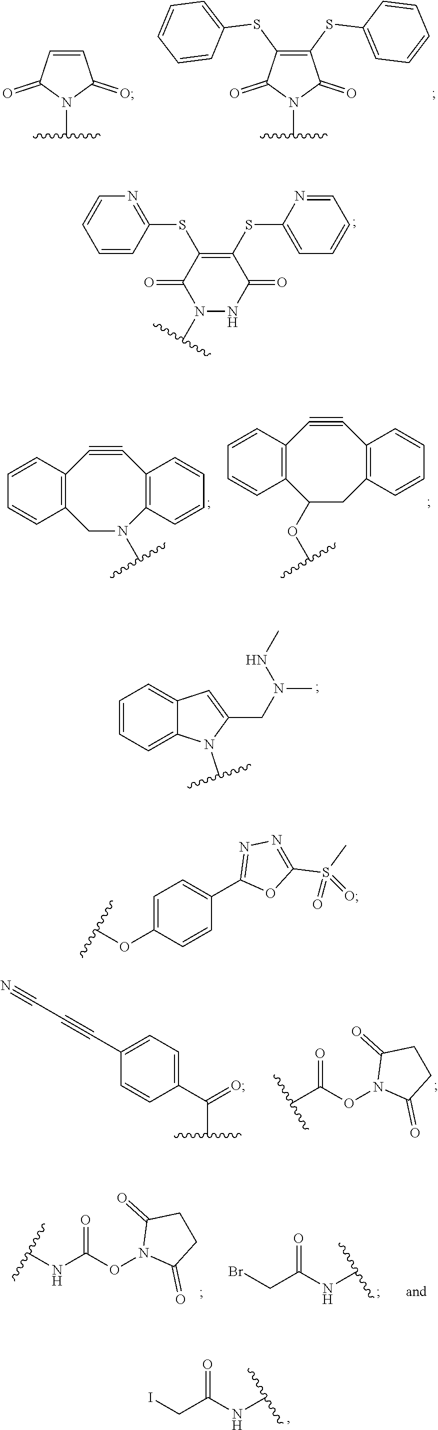

wherein [symbol] represents the site of attachment of R¹⁰ to the remainder of the linker.

In another embodiment of the present invention, R¹⁰ is

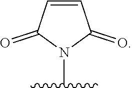 [SMILES: *N1C(=O)C=CC1=O]

In a preferred embodiment of the present invention, the linker-R¹⁰ comprises a motif of the following structure:

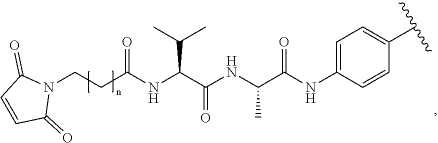

wherein n is an integer from 1 to 6 and [symbol] represents the site of coupling of the linker to the compound or to a functional group that couples the linker to the compound.

In an embodiment of the present invention, Ama comprises a 4,5-dihydroxyleucine moiety and the linker is coupled to the compound via a cyclic acetal obtained from reaction of the hydroxyl groups of the 4,5-dihydroxyisoleucine moiety with a ketone moiety on the linker.

In an embodiment of the present invention, n is 1.

In an embodiment of the present invention, the linker is coupled to the compound via reaction of R¹ with a reactive group thereto on the linker. In another embodiment of the present invention, the reactive group is a para-nitrophenyl ester.

In an embodiment of the present invention, n is 4.

In another aspect, the present invention relates to a conjugate comprising a target-binding moiety conjugated to a compound or a compound-linker construct as described herein.

In an embodiment of the present invention, the conjugate comprises the compound-linker construct, R¹⁰ is a thiol-reactive group, the target-binding moiety comprises an engineered cysteine residue and the compound-linker construct is conjugated to the target-binding moiety via a moiety resulting from the reaction of the thiol of the engineered cysteine residue with R¹⁰. In an embodiment of the present invention, the engineered cysteine residue is selected from the group consisting of heavy chain 118Cys, heavy chain 239Cys, and heavy chain 265Cys. In another embodiment of the present invention, the engineered cysteine residue is heavy chain 265Cys.

In an embodiment of the present invention, the target-binding moiety is an antibody, an antigen-binding fragment thereof or an antibody-like protein. In another embodiment of the present invention, the target-binding moiety is an anti-HER2 antibody.

In another aspect, the present invention relates to a pharmaceutical composition comprising a compound of the present invention or a conjugate of the present invention and a pharmaceutically acceptable carrier.

In another aspect, the present invention relates to a use of an effective amount of a compound of the present invention, or a conjugate of the present invention for treatment of a disease associated with cells presenting a target in a subject in need thereof, wherein the target-binding moiety is specific for the target. In another aspect, the present invention relates to a use of an effective amount of a compound of the present invention, or a conjugate of the present invention for preparation of a medicament for treatment of a disease associated with cells presenting a target in a subject in need thereof, wherein the target-binding moiety is specific for the target.

In an embodiment of the present invention, the target-binding moiety is an anti-HER2 antibody and the disease is HER2-positive breast cancer.

In another aspect, the present invention relates to a use of an effective amount of a compound of the present invention, or a conjugate of the present invention for treatment of cancer in a subject in need thereof. In another aspect, the present invention relates to a use of an effective amount of a compound of the present invention, or a conjugate of the present invention for preparation of a medicament for treatment of cancer in a subject in need thereof.

In another aspect, the present invention relates to a compound of the present invention, or a conjugate of the present invention for use to treat a disease associated with cells presenting a target in a subject, wherein the target-binding moiety is specific for the target.

In an embodiment of the present invention, the target-binding moiety is an anti-HER2 antibody and the disease is HER2-positive breast cancer.

In another aspect, the present invention relates to a compound of the present invention, or a conjugate of the present invention for use to treat cancer in a subject.

In another aspect, the present invention relates to a method of treating a disease associated with cells presenting a target in a subject in need thereof, the method comprising administering an effective amount of a compound of the present invention, or a conjugate of the present invention to the subject, wherein the target-binding moiety is specific for the target.

In an embodiment of the present invention, the target-binding moiety is an anti-HER2 antibody and the disease is HER2-positive breast cancer.

In another aspect, the present invention relates to a method of treating cancer in a subject in need thereof, the method comprising administering an effective amount of a compound of the present invention, or a conjugate of the present invention to the subject.

Other features and advantages of the present invention will become apparent from the following detailed description. It should be understood, however, that the detailed description and the specific examples, while indicating embodiments of the invention, are given by way of illustration only and the scope of the claims should not be limited by these embodiments, but should rather be given the broadest interpretation consistent with the description as a whole.

### BRIEF DESCRIPTION OF THE DRAWINGS

The embodiments of the invention will now be described in greater detail with reference to the attached drawings, in which:

FIG. 1 shows the ¹H NMR (upper spectrum) and COSY45 (lower spectrum) of N₃-Pro-amanitin (400 MHz, CD₃OD).

FIG. 2 shows the ¹H NMR (upper spectrum) and COSY45 (lower spectrum) of CN-Pro-amanitin (400 MHz, CD₃OD).

FIG. 3 shows the ¹H NMR (upper spectrum) and COSY45 (lower spectrum) of SAcm-Pro-amanitin (400 MHz, CD₃OD).

FIG. 4 shows the ¹H NMR (upper spectrum) and COSY45 (lower spectrum) of SH-Pro-amanitin (600 MHz, CD₃OD).

FIG. 5 shows the ¹H NMR (upper spectrum) and COSY45 (lower spectrum) of MeOCONH-Pro-amanitin (400 MHz, CD₃OD).

FIG. 6 shows the ¹H NMR (upper spectrum) and COSY45 (lower spectrum) of NH₂-Pro-amanitin (600 MHz, CD₃OD).

FIG. 7 shows the ¹H NMR (upper spectrum) and COSY45 (lower spectrum) of a Boc-Gdn-Pro-amanitin (600 MHz, CD₃OD).

FIG. 8 shows the ¹H NMR (upper spectrum) and COSY45 (lower spectrum) of a Gdn-Pro-amanitin (600 MHz, CD₃OD).

FIG. 9 shows circular dichroism (CD) spectra of α-amanitin in comparison to the synthetic amanitins containing analogs of hydroxyproline (Hyp) in methanol (MeOH).

FIG. 10 shows CD spectra of α-amanitin in comparison to the synthetic amanitins containing analogs of Hyp in MeOH/(0.1% formic acid (FA) in H₂O), 10:1 at pH 4.5.

FIG. 11 shows results of a 3-(4,5-dimethylthiazol-2-yl)-2,5-diphenyltetrazolium bromide (MTT) colorimetric assay for evaluation of the toxicity of the azido, cyano and Acm-S-proline synthetic analogs of amanitin against Chinese hamster ovary (CHO) cells compared to α-amanitin.

FIG. 12 shows the results of an MTT colorimetric assay for evaluation of the toxicity of the mercapto, MeOCONH, amino, Boc-Gdn and Gdn-proline synthetic analogs of amanitin against CHO cells compared to α-amanitin.

FIG. 13 shows IC₅₀ curves and values for a viability assay of amanitin analogs NH₃-Pro-amanitin and NH₂-Pro-amanitin (left); SH-Pro-amanitin and CN-Pro-amanitin (center); and Gdn-Pro-amanitin and MeOCONH-Pro-amanitin (right) in comparison to α-amanitin against HEK293 cells (normal cells, no overexpression of OATP1B3).

FIG. 14 shows IC₅₀ curves and values for a viability assay of amanitin analogs NH₃-Pro-amanitin and NH₂-Pro-amanitin (left); SH-Pro-amanitin and CN-Pro-amanitin (center); and Gdn-Pro-amanitin and MeOCONH-Pro-amanitin (right) in comparison to α-amanitin against HEK293-OATP1B3 cells (overexpressing OATP1B3).

FIG. 15 shows IC₅₀ curves obtained for amanitin analogs CN-Pro-amanitin, SH-Pro-amanitin, NH₂-Pro-amanitin and Gdn-Pro-amanitin in comparison to α-amanitin in an in vitro transcription assay using the HeLaScribe® nuclear extract (n=3).

FIG. 16 shows characteristic inhibition of transcription by α-amanitin in comparison to its analogs CN-Pro-amanitin, SH-Pro-amanitin, NH₂-Pro-amanitin and Gdn-Pro-amanitin under varied concentrations (n=3); [³²P]-autoradiogram of transcription runoff assay resolved by 8% denaturing PAGE (left); and IC₅₀ curves of α-amanitin and its analogs (right).

FIG. 17 shows characteristic inhibition of transcription by α-amanitin (right hand side on each row) in comparison to its analogs N₃-Pro-amanitin, SAcm-Pro-amanitin and GdnBoc-Pro-amanitin (left hand side on each row) at [I]=1.0 μM (n=3). [³²P]-autoradiogram of transcription runoff assay resolved by 8% denaturing PAGE.

FIG. 18 (upper image) shows a schematic of the H-bonding interactions between the hydroxyl group of Hyp in α-amanitin and Pol II (upper schematic). The oxygen of the OH can accept a proton from Glu822 and the proton of the OH acts as an H-bond donor to His1085. FIG. 18 (lower image) shows a schematic of the expected interactions between the ammonium group of the NH₂-Pro-amanitin analog and Pol II. While not wishing to be limited by theory, the proton from the NH₃⁺ group may form a salt bridge with Glu822, and another proton can act as an H-bond donor to His1085. The same concept could be proposed for the Gdn-Pro-amanitin analog. Hydrogen bonds and salt bridges are shown with dashed lines.

FIG. 19 shows the results of the in vitro cell-based assay of ADCs of CN-Pro-amanitin and NH₂-Pro-amanitin against SK-BR-3 (HER2⁺) cells in comparison to the ADC of α-amanitin.

FIG. 20 shows the results of the in vitro cell-based assay of ADCs of CN-Pro-amanitin and NH₂-Pro-amanitin against SK-OV-3 (HER2⁺) cells in comparison to the ADC of α-amanitin.

FIG. 21 shows the results of the in vitro cell-based assay of ADCs of CN-Pro-amanitin and NH₂-Pro-amanitin against JIMT-1 (HER2⁺) cells in comparison to the ADC of α-amanitin.

FIG. 22 shows the results of the in vitro cell-based assay of ADCs of CN-Pro-amanitin and NH₂-Pro-amanitin against MDA-MB-231 (HER2⁻) cells in comparison to the ADC of α-amanitin.

### DETAILED DESCRIPTION

### I. Definitions

Unless otherwise indicated, the definitions and embodiments described in this and other sections are intended to be applicable to all embodiments and aspects of the invention herein described for which they would be understood to be suitable by a person skilled in the art.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The term “alkyl” as used herein, whether it is used alone or as part of another group, means straight or branched chain, saturated alkyl groups. The number of carbon atoms that are possible in the referenced alkyl group are indicated by the numerical prefix “Cₙ₁₋ₙ₂”. For example, the term C₁₋₆alkyl means an alkyl group having 1, 2, 3, 4, 5 or 6 carbon atoms.

The term “alkylene” as used herein, means a straight or branched chain, bivalent form of an alkane. The number of carbon atoms that are possible in the referenced alkylene group are indicated by the numerical prefix “Cₙ₁₋ₙ₂”. For example, the term C₂₋₁₆alkylene means an alkylene group having 2, 3, 4, 5, 6, 7, 8, 9, 10, 11, 12, 13, 14, 15 or 16 carbon atoms.

The term “aryl” as used herein, whether it is used alone or as part of another group, refers to cyclic groups that contain at least one aromatic ring. In an embodiment, the aryl group contains from 6, 9, 10 or 14 atoms, such as phenyl, naphthyl, indanyl or anthracenyl.

The term “subject” as used herein includes all members of the animal kingdom including mammals. In an embodiment of the present invention, the subject is a human.

The term “pharmaceutically acceptable” as used herein means compatible with the treatment of subjects, for example, mammals such as humans.

The terms “to treat”, “treating” and “treatment” as used herein and as is well understood in the art, mean an approach for obtaining beneficial or desired results, including clinical results. Beneficial or desired clinical results include, but are not limited to alleviation or amelioration of one or more symptoms of a disease (e.g. cancer), diminishment of the extent of the disease, stabilization (i.e. not worsening) of the disease, delay or slowing of the progression of the disease, amelioration or palliation of the state of the disease, diminishment of the recurrence of the disease and/or remission (whether partial or total) of the disease, whether detectable or undetectable. To “treat”, “treating” and “treatment” as used herein also include prophylactic treatment of the disease. For example, in an embodiment of the present invention, a subject with early stage disease is treated to prevent progression or alternatively a subject in remission is treated to prevent recurrence.

As used herein, the term “effective amount” means an amount effective, at dosages and for periods of time necessary to achieve a desired result. For example, in the context of treating cancer, an effective amount of the compound or conjugate of the present invention is an amount that, for example, reduces the cancer compared to the cancer without administration of the conjugate. Effective amounts may vary according to factors such as the disease state, age, sex and/or weight of the subject. The amount of a given compound or conjugate that will correspond to such an amount will vary depending upon various factors, such as the given compound or conjugate, the pharmaceutical formulation, the type of disease being treated, the identity of the subject being treated, and the like, but can nevertheless be routinely determined by one skilled in the art.

### II. Compounds, Compound-Linker Constructs and Conjugates

Herein the synthesis and biochemical evaluation of several derivatives of α-amanitin that contain analogs of its trans-hydroxyproline residue is reported. Surprisingly, α-amanitin is exquisitely intolerant of substitution of the hydroxyl group on proline. However, the reduced but still substantial inhibitory activity of some α-amanitin analogs show the potential of these analogs for therapeutic applications, for example, used as payload for conjugates such as ADCs.

Accordingly, in one aspect the present invention relates to a compound of formula Ama-R¹, wherein Ama is an amatoxin, and R¹ is a trans-4-substituent on the proline residue of the amatoxin, wherein R¹ is not OH.

The amatoxin can be any suitable amatoxin. Amatoxins specifically inhibit the DNA-dependent RNA polymerase II of mammalian cells, and thereby also the transcription and protein biosynthesis of the affected cells. Inhibition of transcription in a cell causes stop of growth and proliferation. Though not covalently bound, the complex between amanitin and RNA-polymerase II is very tight (KD=3 nM). Dissociation of amanitin from the enzyme is a very slow process, thus making recovery of an affected cell unlikely. When the inhibition of transcription lasts sufficiently long, the cell will undergo programmed cell death (apoptosis). The term “amatoxin” as used herein includes all cyclic peptides composed of 8 amino acids as isolated from the genus Amanita and described, for example, by Wieland, T. and Faulstich, H. in CRC Critical Reviews in Biochemistry 1978, 5 (3), 185-260 as well as chemical derivatives thereof, and further all semisynthetic analogs thereof, further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin. As used herein, a “derivative” of a compound refers to a species having a chemical structure that is similar to the compound, yet containing at least one chemical group not present in the compound and/or deficient of at least one chemical group that is present in the compound. The compound to which the derivative is compared is known as the “parent” compound. Typically, a “derivative” may be produced from the parent compound in one or more chemical reaction steps. As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%, more preferably at least 40%, and more preferably at least 50%) of the activity of the parent compound. Thus, an “analogue of an amatoxin”, as it is used herein, refers to a compound that is structurally related to any one of α-amanitin, β-amanitin, γ-amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, and more preferably at least 50%) of the inhibitory activity against mammalian RNA polymerase II as compared to at least one of α-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of α-amanitin, β-amanitin, γ-amanitin, F-amanitin, amanin, amaninamide, amanullin, or amanullinic acid. The inhibitory activity might be measured by determining the concentration at which 50% inhibition occurs (IC₅₀ value). The inhibitory activity against mammalian RNA polymerase II can be determined indirectly by measuring the inhibitory activity on cell proliferation. A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes. According to some embodiments of the present invention, the amatoxin can be selected from the group consisting of α-amanitin, β-amanitin, amanin, amaninamide and analogues, derivatives and salts thereof. Functionally, amatoxins are defined as peptides or depsipeptides that inhibit mammalian RNA polymerase II. Preferred amatoxins are those with a functional group (e.g. a carboxylic group, an amino group, a hydroxy group, a thiol or a thiol-capturing group) that can be reacted with linker molecules or target-binding moieties as defined below. In the context of the present invention, the term “amanitins” particularly refers to bicyclic structures that are based on an aspartic acid or asparagine residue in position 1, a proline residue, other than a hydroxyproline residue in position 2, an isoleucine, hydroxyisoleucine or dihydroxyisoleucine in position 3, a tryptophan or hydroxytryptophan residue in position 4, glycine residues in positions 5 and 7, an isoleucine residue in position 6, and a cysteine residue in position 8, such as a derivative of cysteine that is oxidized to a sulfoxide or sulfone derivative, and furthermore includes all chemical derivatives thereof; further all semisynthetic analogues thereof; further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase II. In an embodiment of the present invention, the amatoxin is an α-amanitin, β-amanitin, γ-amanitin, ε-amanitin, amanullin, amanullinic acid, amanin, amaninamide, γ-amanin or γ-amaninamide or a chemical derivative thereof.

In an embodiment of the present invention, Ama-R¹ is a compound of Formula I.

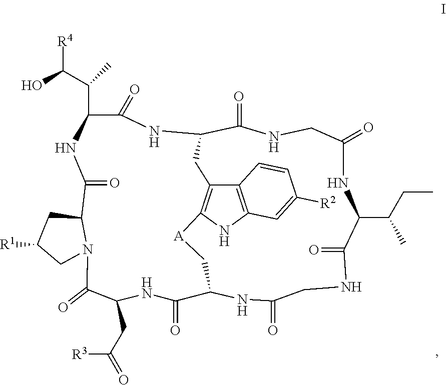

wherein
   R¹ is as defined for Ama-R¹;
   R² is H or OH;
   R³ is NHR⁵ or OR⁶;
   R⁴ is H, CH₃, CH₂OH or CH₂OC(O)R⁷;
   R⁵ is selected from H, NHR⁸, NHOR⁹, C₁₋₆alkyl and aryl;
   R⁶, R⁷, R⁸ and R⁹ are each independently selected from H, C₁₋₆alkyl and aryl; and
   A is S, (R)—SO or SO₂.

In an embodiment of the present invention, R³ is NHR⁵. In another embodiment of the present invention, R⁵ is H (i.e. R³ is NH₂). In another embodiment of the present invention, R⁵ is NHR⁸. In another embodiment of the present invention, R⁵ is NHOR⁹. In another embodiment of the present invention, R⁵ is C₁₋₆alkyl. In another embodiment of the present invention, R⁵ is methyl. In another embodiment of the present invention, R⁵ is aryl.

In an embodiment of the present invention, R³ is OR⁶.

In an embodiment of the present invention, R⁴ is H. In another embodiment of the present invention R⁴ is CH₃. In another embodiment of the present invention R⁴ is CH₂OH. In another embodiment of the present invention R⁴ is or CH₂OC(O)R⁷.

In another embodiment of the present invention, R⁶, R⁷, R⁸ and R⁹ are each H. In another embodiment of the present invention, R⁶, R⁷, R⁸ and R⁹ are each C₁₋₆alkyl. In another embodiment of the present invention, R⁶, R⁷, R⁸ and R⁹ are each methyl. In another embodiment of the present invention, R⁶, R⁷, R⁸ and R⁹ are each aryl. In another embodiment of the present invention, R⁶ is H. In another embodiment of the present invention, R⁶ is C₁₋₆alkyl. In another embodiment of the present invention, R⁶ is methyl. In another embodiment of the present invention, R⁶ is aryl. In another embodiment of the present invention, R⁷ is H. In another embodiment of the present invention, R⁷ is C₁₋₆alkyl. In another embodiment of the present invention, R⁷ is methyl. In another embodiment of the present invention, R⁷ is aryl. In another embodiment of the present invention, R⁸ is H. In another embodiment of the present invention, R⁸ is C₁₋₆alkyl. In another embodiment of the present invention, R⁸ is methyl. In another embodiment of the present invention, R⁸ is aryl. In another embodiment of the present invention, R⁹ is H. In another embodiment of the present invention, R⁹ is C₁₋₆alkyl. In another embodiment of the present invention, R⁹ is methyl. In another embodiment of the present invention, R⁹ is aryl.

In an embodiment of the present invention, Ama-R¹ is a compound of Formula I(a):

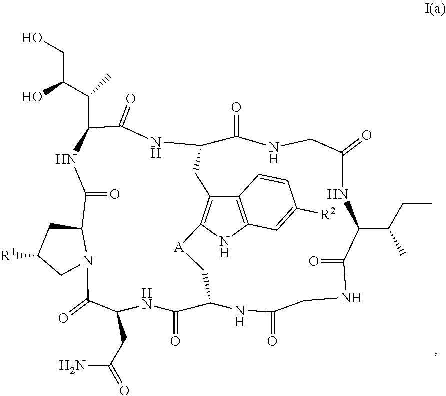

wherein
   R¹ is as defined for Ama-R¹;
   R² is H or OH; and
   A is S, (R)—SO or SO₂.

In an embodiment of the present invention, R² is H. In another embodiment of the present invention, R² is OH.

In an embodiment of the present invention, A is S or (R)—SO. In another embodiment of the present invention, A is S. In a further embodiment of the present invention, A is (R)—SO. In another embodiment of the present invention, A is SO₂.

In an embodiment of the present invention, R¹ is —NH₂, —NC(NH₂)₂, —CN or —SH. In another embodiment of the present invention, R¹ is —CN or —NH₂. In a further embodiment of the present invention, R¹ is —CN. In another embodiment of the present invention, R¹ is —NH₂. In another embodiment of the present invention, R¹ is —NC(NH₂)₂. In another embodiment of the present invention, R¹ is —SH.

The compounds of the present invention may, for example, be coupled to a linker to provide a compound-linker construct that may, for example, be used in the manufacture of conjugates which may, for example, be useful in the treatment of diseases such as cancer.

Accordingly, another aspect of the present invention relates to a compound-linker construct comprising a compound of the present invention (e.g. a compound of formula Ama-R, Formula I or Formula I(a) as described herein) coupled to a linker, wherein the linker comprises a reactive group R¹⁰ for conjugating the compound-linker construct to a target-binding moiety.

The linker can be any suitable linker. For example, it will be appreciated by a person skilled in the art that amatoxins are relatively non-toxic when coupled to a biomolecule carrier such as a target-binding moiety, and advantageously only exert their cytotoxic activity after internalization in the target cells. Accordingly, a conjugate comprising the target-binding moiety conjugated to the compound coupled to the linker is advantageously substantially stable in the plasma after administration and a suitable linker may desirably allow, for example, for substantially releasing the compound subsequent to internalization in the target cells.

In an embodiment of the present invention, the linker is stable linker. The term “stable linker” as used herein refers to a linker that typically releases the compound after the target-binding moiety to which it is conjugated is degraded intracellularly, for example, in the lysosomes. In other words, the linker is substantially stable in an intracellular reducing environment and in the presence of enzymes such as lysosomal peptidases (e.g. Cathepsin B). In an embodiment of the present invention, the stable linker is devoid of an enzyme-cleavable structure (e.g. a dipeptide sequence cleavable by Cathepsin B) and/or a disulfide group. In another embodiment of the present invention, the linker is of the structure -L-, wherein L is C₂₋₁₆alkylene or (CH₂CH₂O)ₘCH₂CH₂ wherein m is an integer of from 2 to 4; and −represents the sites of attachment to the compound of Ama-R¹, Formula I or Formula I(a) and the reactive group R¹⁰.

In another embodiment of the present invention, the linker is a cleavable linker. The term “cleavable linker” as used herein refers to a linker that is cleavable by an enzyme and/or in a reducing environment. In an embodiment of the present invention, the cleavable linker is cleavable by an intracellular protease. In another embodiment of the present invention, the linker is cleavable by at least one agent selected from the group consisting of a cysteine protease, a metalloproteinase, a serine protease, a threonine protease and an aspartic protease. Such linkers may, for example, comprise a peptide motif cleavable by such an enzyme. In another embodiment of the present invention, the cleavable linker comprises a dipeptide that is valine-citrulline (Val-Cit), phenylalanine-citrulline (Phe-Cit), valine-alanine (Val-Ala), phenylalanine-alanine (Phe-Ala), valine-lysine (Val-Lys) or phenylalanine-lysine (Phe-Lys). In another embodiment of the present invention, the cleavable linker comprises a motif that is valine-alanine (Val-Ala).

In an embodiment of the present invention, the linker further comprises a self-immolating moiety. The term “self-immolating moiety” as used herein, refers to a moiety, which, after enzymatic cleavage of the linker, spontaneously cleaves from the remainder of the compound-linker construct, thereby releasing the compound. In an embodiment of the present invention, the self-immolating moiety is a p-aminobenzyl alcohol (PAB) moiety. In an embodiment of the present invention, the PAB is conjugated to a peptide (e.g. a dipeptide) portion of the linker via the aromatic amine group of the PAB. In another embodiment of the present invention, the PAB is conjugated to the compound via a carbamate group coupled to a primary or secondary amine or in an alternative embodiment of the present invention, the PAB is coupled directly to the compound.

The reactive group R¹⁰ is any reactive group suitable for conjugating the compound-linker construct to an antibody or antigen-binding fragment thereof. In an embodiment of the present invention, R¹⁰ is selected from

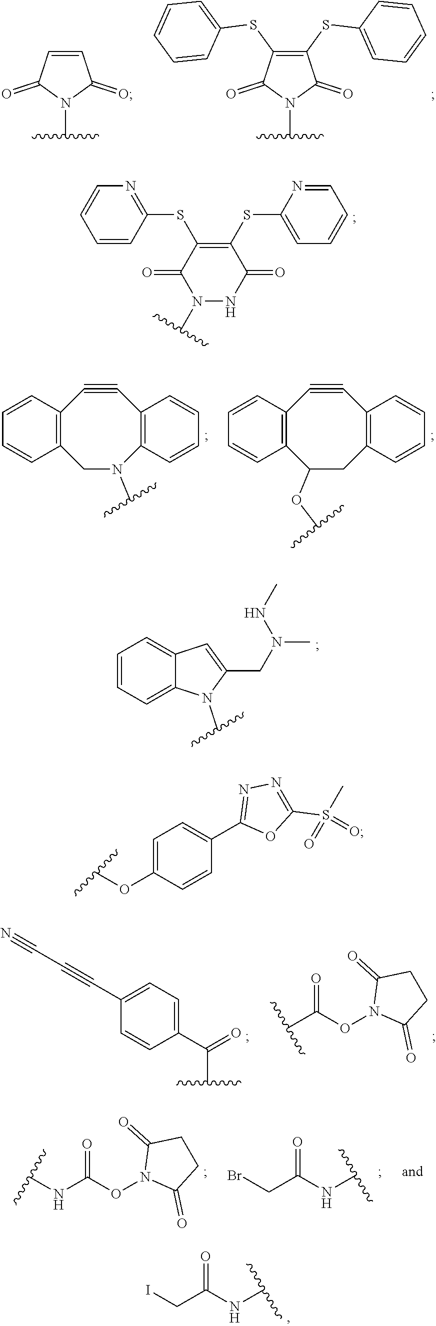

wherein [symbol] represents the site of attachment of R¹⁰ to the remainder of the linker. In another embodiment of the present invention, R¹⁰ is

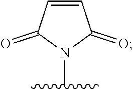 [SMILES: *N1C(=O)C=CC1=O]

i.e. is a maleimide reactive group.

In a preferred embodiment of the present invention, the linker-R¹⁰ comprises a motif of the following structure:

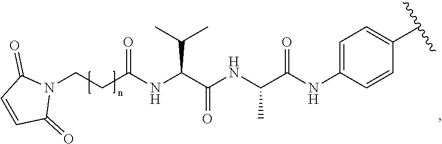

wherein n is an integer from 1 to 6 and [symbol] represents the site of coupling of the linker to the compound or to a functional group that couples the linker to the compound.

The compounds of the present invention are coupled to the linker in any suitable configuration, the selection of which can be made by a person skilled in the art. For example, the location of the coupling may depend, for example, on the identity of Ama, R¹ and/or the linker.

In an embodiment of the present invention, Ama comprises a 4,5-dihydroxyisoleucine moiety and the linker is coupled to the compound via a cyclic acetal obtained from reaction of the hydroxyl groups of the 4,5-dihydroxyisoleucine moiety with a ketone moiety on the linker. In a further embodiment of the present invention, n is 1.

In an embodiment of the present invention, the compound-linker construct is of the following structure:

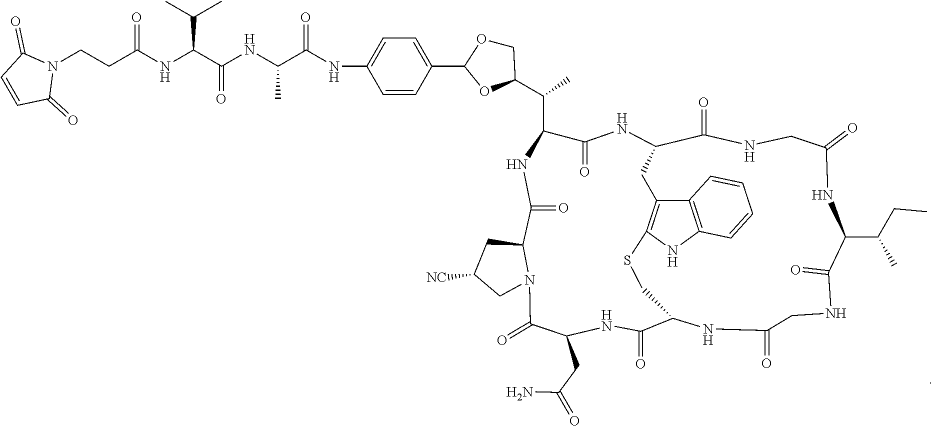

In an alternative embodiment of the present invention, the linker is coupled to the compound (e.g. the compound of Formula I or the compound of Formula I(a)) via reaction of R¹ with a reactive group thereto on the linker. In a further embodiment of the present invention, the reactive group is a para-nitrophenyl ester. In another embodiment of the present invention, n is 4.

In an embodiment of the present invention, the compound-linker construct is of the following structure:

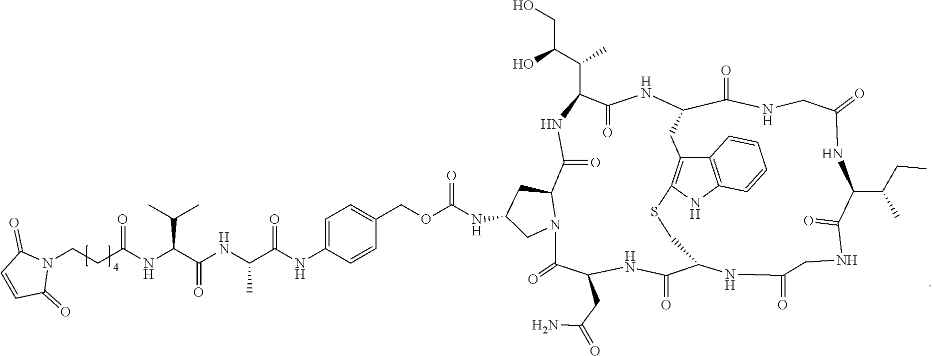

In another aspect, the present invention relates to a conjugate comprising a target-binding moiety conjugated to a compound or a compound-linker construct as described herein. In an embodiment of the present invention, the conjugate comprises the target-binding moiety conjugated to the compound. In another embodiment of the present invention, the conjugate comprises the target-binding moiety conjugated to the compound-linker construct.

The term “target-binding moiety” as used herein refers to any suitable moiety that can specifically bind to a target molecule (e.g. protein) or epitope. In an embodiment of the present invention, the target-binding moiety is an antibody, antigen-binding fragment thereof or an antibody-like protein. The term “antigen-binding fragment thereof” as used herein means that the fragment of the antibody comprises at least a functional antigen-binding domain. The term “antibody-like protein” as used herein refers to a protein that is not strictly an antibody but has the capability of binding to a target molecule (e.g. protein) or epitope. The “antibody-like protein” is a protein that has been engineered (e.g. by mutagenesis of Ig loops) to specifically bind to the target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. In an embodiment of the present invention, the scaffold protein is a small globular protein. The scaffold of antibody-like proteins can be based on for example, without limitation, affilin proteins, affibodies, anti-calins, lipocalins, ubiquitin, leucine-rich repeat proteins, and designed ankyrin repeat proteins (see, for example: Binz et al., “Engineering novel binding proteins from nonimmunoglobulin domains” Nat Biotechnol. 2005, 23:10, 1257-68). Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins. The antibody or antigen-binding fragment thereof can be from any suitable immunoglobulin type (e.g. IgG, IgE, IgM, IgD, IgA and/or IgY), class (e.g., IgG1, IgG2, IgG3, IgG4, IgA1 and/or IgA2) or subclass. Suitable antibodies and/or antigen-binding fragments thereof may include but are not limited to polyclonal, monoclonal, monovalent, bispecific, heteroconjugate, multispecific, human, humanized (e.g. CDR-grafted), deimmunized, and/or chimeric antibodies, single chain antibodies (e.g. scFv), Fab fragments, F(ab′)₂ fragments, fragments produced by a Fab expression library, diabodies or tetrabodies, nanobodies, anti-idiotypic (anti-Id) antibodies (including, but not limited to anti-Id antibodies to antibodies of the present invention), and epitope-binding fragments of any of the above.

In some embodiments of the present invention, the antigen-binding fragments are human antigen-binding antibody fragments and include, but are not limited to, Fab, Fab′ and F(ab′)₂, Fd, single-chain Fvs (scFv), single-chain antibodies, disulfide-linked Fvs (dsFv) and fragments comprising either a VL or VH domain. Antigen-binding antibody fragments, including single-chain antibodies, may comprise the variable domain(s) alone or in combination with the entirety or a portion of the following: hinge region, CL, CH1, CH2, and CH3 domains. Also included are antigen-binding fragments also comprising any combination of variable domain(s) with a hinge region, CL, CH1, CH2, and CH3 domains. The antibody, antigen-binding fragment thereof or antibody-like protein may be from any animal origin including birds and mammals. For example, in an embodiment of the present invention, the antibody, antigen-binding fragment thereof or antibody-like protein is from human, rodent (e.g. mouse, rat, guinea pig, or rabbit), chicken, pig, sheep, goat, camel, cow, horse, donkey, cat, or dog origin. In another embodiment of the present invention, the antibody, antigen-binding fragment thereof or antibody-like protein is of human or murine origin. The term “human” as used herein in reference to antibodies includes antibodies having the amino acid sequence of a human immunoglobulin and also includes antibodies isolated from human immunoglobulin libraries or from animals transgenic for one or more human immunoglobulin and that do not express endogenous immunoglobulins, as described for example in U.S. Pat. No. 5,939,598.

The term “specifically bind” as used herein in reference to a target-binding moiety specifically binding to a target molecule or epitope means that it has a dissociation constant K_{D} to the target molecule or epitope of at most about 100 μM. In an embodiment of the present invention, K_{D} is about 100 PM or lower, about 50 μM or lower, about 30 μM or lower, about 20 μM or lower, about 10 μM or lower, about 5 μM or lower, about 1 μM or lower, about 900 nM or lower, about 800 nM or lower, about 700 nM or lower, about 600 nM or lower, about 500 nM or lower, about 400 nM or lower, about 300 nM or lower, about 200 nM or lower, about 100 nM or lower, about 90 nM or lower, about 80 nM or lower, about 70 nM or lower, about 60 nM or lower, about 50 nM or lower, about 40 nM or lower, about 30 nM or lower, about 20 nM or lower or about 10 nM or lower, about 1 nM or lower, about 900 pM or lower, about 800 pM or lower, about 700 pM or lower, about 600 pM or lower, about 500 pM or lower, about 400 pM or lower, about 300 pM or lower, about 200 pM or lower, about 100 pM or lower, about 90 pM or lower, about 80 pM or lower, about 70 pM or lower, about 60 pM or lower, about 50 pM or lower, about 40 pM or lower, about 30 pM or lower, about 20 pM or lower or about 10 pM or lower.

The terms “target molecule” and “target epitope” as used herein refer to an antigen and an epitope of an antigen, respectively, that is specifically bound by the antibody, antigen-binding fragment thereof or antibody-like protein. The term “epitope” as used herein, also sometimes referred to in the art as an “antigenic determinant”, refers to a molecular structure to which, in the context of an adaptive immune response, antibodies or T cell receptors are directed and/or generated, and/or which can elicit a specific immune response. In the context of antibodies or antigen-binding fragments thereof, the term “epitope” as used herein relates to the specific molecular structure on the antigen to which the antigen-recognition site or paratop of the antibody or antigen-binding fragment thereof, binds. In an embodiment of the present invention, the target molecule or target epitope is associated with cancer, non-cancerous neoplasms, an autoimmune disease or an inflammatory disease. In an embodiment of the present invention, the target molecule or epitope is associated with cancer or non-cancerous neoplasms. In another embodiment of the present invention, the target molecule or epitope associated with cancer or non-cancerous neoplasms is present on the surface of one or more tumor cell types or tumor-associated cells in an increased concentration and/or in a different steric configuration as compared to the surface of non-tumor cells. In an embodiment of the present invention, the target molecule or target epitope is associated with cancer. In a further embodiment of the present invention, the target molecule or epitope associated with cancer is an epitope of human epidermal growth receptor 2 (HER2), prostate-specific membrane antigen (PSMA), CD20, CD269, sialyl Lewis^{X}, HER-2/neu or epithelial cell adhesion molecule (EpCAM). In a further embodiment of the present invention, the target molecule or epitope associated with cancer is an epitope of human epidermal growth receptor 2 (HER2). In another embodiment of the present invention, the target-binding moiety is an anti-HER2 antibody. In an embodiment of the present invention, the target molecule or target epitope is associated with autoimmune disease. In another embodiment of the present invention, the target molecule or epitope associated with autoimmune disease is preferentially expressed on cells involved in an autoimmune disease. In an embodiment of the present invention, the target molecule or target epitope is associated with inflammatory disease. In another embodiment of the present invention, the target molecule or epitope associated with inflammatory disease is preferentially expressed on cells involved in an inflammatory disease.

In an embodiment of the present invention wherein the conjugate comprises the compound-linker construct, R¹⁰ is a thiol-reactive group, the target-binding moiety comprises an engineered cysteine residue and the compound-linker construct is conjugated to the target-binding moiety via a moiety resulting from the reaction of the thiol of the engineered cysteine residue with R¹⁰.

The term “engineered cysteine residue” as used herein refers to a cysteine residue that is introduced into the peptide sequence of the target-binding moiety that is generally not present in the native peptide sequence of the target-binding moiety. Such cysteine residues are available for conjugation but desirably do not substantially affect immunoglobulin folding, antibody assembly, antigen binding and/or Fc domain effector functions. For example, the engineered cysteine residue can take the place of the amino acid that naturally occurs at a given position in the peptide sequence; i.e. in an embodiment of the present invention, the engineered cysteine residue is a cysteine substitution. Engineered cysteine residues can be introduced into the peptide sequence of the target-binding moiety through suitable techniques such as site-directed mutagenesis, the selection of which can be made by a person skilled in the art. In an embodiment of the present invention, the target-binding moiety comprises one engineered cysteine residue. In another embodiment of the present invention, the target-binding moiety comprises greater than one engineered cysteine residue, for example, two engineered cysteine residues. For example, antibodies wherein the amino acid aspartic acid at position 265 has been exchanged to cysteine (D265) contains two introduced cysteines at each chain of the Fc region, which can serve as conjugation sites for the compound-linker construct to produce a conjugate with DAR=2.

In an embodiment of the present invention, the engineered cysteine residue is selected from the group consisting of heavy chain 118Cys, heavy chain 239Cys, and heavy chain 265Cys. In another embodiment of the present invention, the engineered cysteine residue is heavy chain 265Cys.

### III. Uses

In another aspect, the present invention relates to a composition comprising a compound or conjugate of the present invention and a carrier. In an embodiment of the present invention, the composition comprises the compound of the present invention and the carrier. In another embodiment of the present invention, the composition comprises the conjugate of the present invention and the carrier. The compounds and/or conjugates of the present invention are optionally formulated into pharmaceutical compositions for administration in a biologically compatible form, for example, a form suitable for administration to or for use in subjects in vivo. Accordingly, in another aspect, the present invention relates to a pharmaceutical composition comprising a compound or conjugate of the present invention and a pharmaceutically acceptable carrier. In an embodiment of the present invention, the pharmaceutical composition comprises the compound of the present invention and the pharmaceutically acceptable carrier. In another embodiment of the present invention, the pharmaceutical composition comprises the conjugate of the present invention and the pharmaceutically acceptable carrier.

The compound or conjugate of the present invention can be administered to a subject or used in a variety of forms depending on the selected route of administration or use, as will be understood by a person skilled in the art. For example, the compound or conjugate of the present invention is suitably administered to the subject or for use parenterally; i.e. taken into the body or administered or used in a manner other than through the gastrointestinal tract. In an embodiment of the present invention, the compound or conjugate of the present invention is administered or for use as an injectable or infusion. Injectables can be formulated in the form of ampules and/or as a ready-for-use injectable such as a ready-to-use syringe, a single-use syringe and/or in a puncturable flask for multiple withdrawal. In another embodiment of the present invention, the injectable is administered or for use in the form of a subcutaneous (s.c.), intramuscular (i.m.), intravenous (i.v.) or intracutaneous (i.c.) injection. In an embodiment of the present invention, the infusion is in the form of an isotonic solution, fatty emulsion, liposomal formulation or micro-emulsion. A person skilled in the art would know how to prepare suitable formulations. In some embodiments of the present invention, the injectable or infusion formulation is in the form of a concentrate which can be dissolved or dispersed with aqueous isotonic diluents. Injectable formulations can also be administered or for use in the form of a permanent infusion e.g. via a mini-pump.

In some embodiments of the present invention, the parenteral formulation further comprises albumin, plasma, expander, surface-active substances, organic diluents, pH-influencing substances, complexing substances, polymeric substances or combinations thereof, for example to influence the adsorption of the compound or conjugate of the present invention to proteins or polymers and/or to reduce the adsorption of the compound or conjugate of the present invention to materials like injection instruments or packaging-materials, for example, plastic or glass.

In some embodiments of the present invention, adjuvants and carriers in the pharmaceutical compositions of the present invention formulated as parenterals are one or more of aqua sterilisata (sterilized water), pH value influencing substances (for example, suitable organic or inorganic acids or bases and salts thereof), buffering substances for adjusting pH values, substances for isotonization (for example, sodium chloride, sodium hydrogen carbonate, glucose or fructose), surfactants (for example, partial esters of fatty acids of polyoxyethylene sorbitans such as a Tween™ surfactant or fatty acid esters of polyoxyethylenes such as a Cremophor™ surfactant), fatty oils (such as soybean oil or castor oil), synthetic esters of fatty acids (for example, ethyl oleate or isopropyl myristate), polymeric adjuvants (for example, gelatine, dextran or polyvinylpyrrolidone), additives which increase the solubility of organic solvents (for example, propylene glycol, ethanol or N,N-dimethylacetamide) complex forming substances (for example, citrate or urea), preservatives (for example, benzoic acid hydroxypropyl ester, benzoic acid methyl ester or benzyl alcohol), antioxidants (for example, sodium sulfite) and stabilizers (for example, ethylenediaminetetraacetic acid, EDTA).

In another aspect, the present invention relates to all uses for the compounds, compound-linker constructs and conjugates of the present invention, including use in therapeutic methods, diagnostic assays and as research tools whether alone or in combination with another active pharmaceutical ingredient.

Derivatives of α-amanitin that contain analogs of its trans-hydroxyproline (Hyp) residue analogs were characterized by in vitro transcription assays and cytotoxicity assays. Surprisingly, α-amanitin is exquisitely intolerant of substitution of the hydroxyl group on proline. However, the reduced but still substantial inhibitory activity of some α-amanitin analogs show the potential of these analogs for therapeutic applications, for example, used as payload for conjugates such as antibody-drug conjugates (ADCs). The combination of being a poor substrate for OATP1B3 transporters whilst retaining inhibitory activity of amanitin to a certain degree might, for example help to develop conjugates with reduced payload-mediated toxicity and/or an improved target-specific effect. Therefore, the compounds and conjugates of the present invention are useful as medicaments. Accordingly, in another aspect the present invention relates to a compound or conjugate of the present invention for use as a medicament. In an embodiment of the present invention, the compound of the present invention is for use as a medicament. In another embodiment of the present invention, the conjugate of the present invention is for use as a medicament.

In another aspect, the present invention relates to a method of treating a disease associated with cells presenting a target in a subject in need thereof, the method comprising administering an effective amount of a conjugate of the present invention to the subject, wherein the target-binding moiety is specific for the target. In another aspect, the present invention relates to a method of treating a disease associated with cells presenting a target in a subject in need thereof, the method comprising administering an effective amount of a compound of the present invention to the subject, wherein the target-binding moiety is specific for the target. In another aspect, the present invention relates to a use of an effective amount of a conjugate of the present invention for treating a disease associated with cells presenting a target in a subject in need thereof, wherein the target-binding moiety is specific for the target. In another aspect, the present invention relates to a use of an effective amount of a compound of the present invention for treating a disease associated with cells presenting a target in a subject in need thereof, wherein the target-binding moiety is specific for the target. In another aspect, the present invention relates to a use of an effective amount of a conjugate of the present invention for preparation of a medicament for treating a disease associated with cells presenting a target in a subject in need thereof, wherein the target-binding moiety is specific for the target. In another aspect, the present invention relates to a use of an effective amount of a compound of the present invention for preparation of a medicament for treating a disease associated with cells presenting a target in a subject in need thereof, wherein the target-binding moiety is specific for the target. In another aspect, the present invention relates to a conjugate of the present invention for use to treat a disease associated with cells presenting a target in a subject, wherein the target-binding moiety is specific for the target. In another aspect, the present invention relates to a compound of the present invention for use to treat a disease associated with cells presenting a target in a subject, wherein the target-binding moiety is specific for the target.

In an embodiment of the present invention, the disease to be treated according to the invention is cancer, a disease associated with non-cancerous neoplasms, an autoimmune disease or an inflammatory disease. The term “cancer” as used herein refers to diseases caused by uncontrolled cell division and/or the ability of cells to metastasize, or to establish new growth in additional sites. The terms “malignant”, “malignancy”, “neoplasm”, “tumor,” “cancer” and variations thereof refer to cancerous cells or groups of cancerous cells. Particular types of cancer include, but are not limited to, skin cancers (e.g., melanoma), connective tissue cancers (e.g., sarcomas), adipose cancers, breast cancers, head and neck cancers, lung cancers (e.g., mesothelioma), stomach cancers, pancreatic cancers, ovarian cancers, cervical cancers, uterine cancers, anogenital cancers (e.g., testicular cancer), kidney cancers, bladder cancers, colon cancers, prostate cancers, central nervous system (CNS) cancers, retinal cancer, blood, neuroblastomas, multiple myeloma, and lymphoid cancers (e.g., Hodgkin's and non-Hodgkin's lymphomas). In an embodiment of the present invention, the cancer is breast cancer. In a further embodiment of the present invention the cancer is HER2-positive breast cancer. In a further embodiment of the present invention, the disease is an autoimmune disease and/or an inflammatory disease. The term “autoimmune disease” can be used interchangeably with the term “autoimmune disorder” to refer to a condition in a subject characterized by cellular, tissue and/or organ injury caused by an immunologic reaction of the subject to its own cells, tissues and/or organs. The term “inflammatory disease” can be used interchangeably with the term “inflammatory disorder” to refer to a condition in a subject characterized by inflammation, such as chronic inflammation. Autoimmune disorders may or may not be associated with inflammation. Moreover, inflammation may or may not be caused by an autoimmune disorder. Thus, the skilled person would appreciate that certain disorders may e.g. be characterized as both autoimmune and inflammatory disorders. Exemplified autoimmune diseases which may be treated according to the invention include systemic Lupus Erythematosus (SLE), Rheumatoid Arthritis (RA), Autoimmune Hemolytic Anaemia (AIHA), or Sjögren's syndrome.

According to one embodiment, the present invention provides pharmaceutical compositions, such as the parenteral formulations as described herein for use in the treatment of a disease, such as cancer, or autoimmune disease as described herein.

In another aspect, the present invention relates to a method of treating cancer in a subject in need thereof, the method comprising administering an effective amount of a conjugate of the present invention to the subject. In another aspect, the present invention relates to a method of treating cancer in a subject in need thereof, the method comprising administering an effective amount of a compound of the present invention to the subject. In another aspect, the present invention relates to a use of an effective amount of a conjugate of the present invention for treating cancer in a subject in need thereof. In another aspect, the present invention relates to a use of an effective amount of a compound of the present invention for treating cancer in a subject in need thereof. In another aspect, the present invention relates to a use of an effective amount of a conjugate of the present disclosure for preparation of a medicament for treating cancer in a subject in need thereof. In another aspect, the present invention relates to a use of an effective amount of a compound of the present disclosure for preparation of a medicament for treating cancer in a subject in need thereof. In another aspect, the present invention relates to a conjugate of the present invention for use to treat a cancer in a subject. In another aspect, the present invention relates to a compound of the present invention for use to treat a cancer in a subject. In an embodiment of the present invention, the cancer is HER2-positive breast cancer.

Treatment methods or uses comprise administering to a subject or use of an effective amount of a compound or conjugate of the present invention, optionally consisting of a single administration or use, or alternatively comprising a series of administrations or uses. For example, the compounds or conjugates of the present invention are administered or used at least once a week. However, in another embodiment of the present invention, the compound or conjugate is administered to the subject or for use from one time per three weeks, or one time per week to once daily for a given treatment or use. The length of the treatment period or use depends on a variety of factors, such as the severity of the disease, the age of the subject, the activity of the compound or conjugate of the present invention and/or a combination thereof. It will also be appreciated by the person skilled in the art that the effective amount of a compound or conjugate used for the treatment or use may increase or decrease over the course of a particular treatment regime or use. Changes in dosage may result and become apparent by standard diagnostic assays known in the art. In some instances, chronic administration or use is required. For example, the compound or conjugate of the present invention is administered or for use in an amount and for a duration suitable to treat the subject.

The compound or conjugate of the present invention may be administered or used alone or in combination with other therapeutic agents useful for treating a disease (e.g. cancer). When administered or for use in combination with other known therapeutic agents, it is an embodiment of the present invention that the compound or conjugate of the present invention is administered or for use contemporaneously with those therapeutic agents. As used herein, the term “contemporaneous” in reference to administration of two substances to a subject or use means providing each of the two substances so that they are both biologically active in the individual at the same time. The exact details of the administration or use will depend on the pharmacokinetics of the two substances in the presence of each other, and can include administration or use of the two substances within a few hours of each other, or even administration or use of one substance within 24 hours of administration or use of the other, if the pharmacokinetics are suitable. Design of suitable dosing regimens is routine for a person skilled in the art. In some embodiments of the present invention, two substances will be administered or for use substantially simultaneously, i.e. within minutes of each other, or in a single composition that includes both substances. It is a further embodiment of the present invention that a combination of the two substances is administered to a subject or for use in a non-contemporaneous fashion.

The dosage of the compound or conjugate of the invention can vary depending on many factors such as the pharmacodynamic properties of the compound or conjugate, the age, health and/or weight of the subject, the nature and/or extent of the symptoms of the disease, the frequency of the treatment or use and the type of concurrent treatment or use, if any, and the clearance rate of the compound or conjugate in the subject. A person skilled in the art can determine the appropriate dosage based on the above factors. In an embodiment of the present invention, the compound or conjugate of the present invention is administered or for use initially in a suitable dosage that is optionally adjusted as required, depending on the clinical response.

In another aspect, the present invention relates to a kit comprising a compound as described herein, a linker as described herein and optionally instructions for preparing a compound-linker construct as described herein from the compound and the linker. In another aspect, the present invention relates to a kit comprising a compound as described herein, a linker as described herein, a target-binding moiety as described herein and optionally instructions for preparing a conjugate as described herein from the compound, the linker and the target-binding moiety. In another aspect, the present invention relates to a kit comprising a compound-linker construct as described herein, a target-binding moiety and optionally instructions for preparing a conjugate as described herein from the compound-linker construct and the target-binding moiety.

The following non-limiting examples are illustrative of the present invention:

### EXAMPLES

### Example 1: Design, Synthesis, and Biochemical Evaluation of Alpha-Amanitin Derivatives Containing Analogs of the Trans-Hydroxyproline Residue

### I. Materials and Methods

(a) General: All reactions were performed under argon atmosphere in flame-dried glassware and dried solvents at room temperature, unless otherwise stated. Controlled temperature reactions were performed using a mineral oil bath and a temperature controlled hot plate (IKA Ceramag Midi). Temperatures below room temperature were achieved in an ice/water bath (0° C.), dry ice/ethylene glycol bath (−20° C.), dry ice/ethanol/ethylene glycol bath (−20° C. to −75° C.) and dry ice/acetone bath (−78° C.). Solvents were removed under reduced pressure using a Büchi rotary evaporator. Anhydrous solvents were prepared by distillation under nitrogen atmosphere or drying over 3 Å or 4 Å molecular sieves for at least 48 hours. Ethers were distilled from sodium in the presence of benzophenone as indicator. Triethylamine, dichloromethane and hexanes were distilled over calcium hydride. Methanol was distilled from magnesium. Dimethylsulfoxide (DMSO) and dimethylformamide (DMF) were dried over 4 Å molecular sieves under argon atmosphere. All reagents and solvents were purchased from Sigma-Aldrich, Alfa Aesar, Acros Organics, Strem Chemicals, Matrix Scientifics, AK Scientific, Oakwood Chemicals or TCI America, unless otherwise stated. Authentic α-amanitin was purchased from Sigma-Aldrich.

(b) Instrumentation: Thin-layer chromatography (TLC) was performed using silica gel 60 F254 precoated aluminum plates (EM Science). Detection of TLC spots was performed using an ultraviolet (UV) lamp at 254 nm, or by staining with p-anisaldehyde, potassium permanganate, ninhydrin or 2,4-dinitrophenylhydrazine, prepared according to literature procedures. Flash column chromatography purifications were performed using silica gel 60 (230-400 mesh, Silicycle, Quebec). Low-resolution mass spectra (LRMS) in electrospray ionization (ESI) mode were obtained from a Bruker Esquire spectrometer. Proton (¹H-NMR), carbon (¹³C-NMR), boron (¹¹B-NMR) and fluorine (¹⁹F-NMR) spectra were obtained using Bruker AV-300 (300 MHz), AV-400inv (400 MHz) and AV600-CRP (600 MHz) spectrometers. Circular Dichroism (CD) spectra were obtained using a Jasco J-815 spectrophotometer.

(c) High performance liquid chromatography (HPLC) purification methods: HPLC chromatograms were generated on an Agilent 1100 system equipped with an auto injector, a fraction collector and a diode array detector. Analytical injections were performed on an Agilent Eclipse XDB C-18 (4.6×250 mm) column with a flow rate of 2 mL/min. The column was fitted with a column guard. In cases of closely-eluting peaks, integration was performed by standard data analysis software package whereby a line was drawn between both peaks and then integration was performed without peak correction. Chromatograms were obtained with a solvent gradient of 0.1% formic acid in water (Solvent B) and 0.1% formic acid in acetonitrile (Solvent A). The solvent gradients were: Gradient A: 0-15 min 5%-40% A, 15-17 min 40%-100% A; 17-19 min 100% A, 19-20 min 100%-5% A, 20-25 min 5% A. Gradient B: 0-18 min 5%-50% A, 18-20 min 50%-100% A; 20-23 min 100% A, 23-25 min 100%-5% A, 25-30 min 5% A. Gradient C: 0-18 min 5%-35% A, 18-21 min 35%-100% A; 21-24 min 100% A, 24-25 min 100%-5% A, 25-30 min 5% A. Gradient D: 0-22 min 5%-35% A, 22-27 min 35%-100% A; 27-32 min 100% A, 32-35 min 100%-5% A, 35-40 min 5% A. Gradient E: 0-22 min 5%-35% A, 22-24 min 35%-100% A; 24-28 min 100% A, 28-30 min 100%-5% A, 30-35 min 5% A. Gradient F: 0-18 min 5%-35% A, 18-25 min 35%-75% A; 25-27 min 75%-100% A, 27-32 min 100% A, 32-34 min 100%-5% A, 34-39 min 5% A. Gradient G: 0-18 min 5%-30% A, 18-28 min 30%-100% A; 28-33 min 100% A, 33-35 min 100%-5% A, 35-40 min 5% A.

(d) Peptide quantification: Quantifications of different peptides were performed using a reported extinction coefficient of 10,000 M⁻¹ cm⁻¹ at the wavelength with the maximum absorbance (290-305 nm), with the exception of α-amanitin that has an extinction coefficient of 13,500 M⁻¹ cm⁻¹. All UV wavelength scans and measurements were performed using a Cary5000 Spectrophotometer and readings were acquired at values of approximately 0.1-0.5 AU. Peptides were purified by HPLC and eluates were lyophilized. The dry compound was re-suspended in a known amount of solvent (0.1% formic acid in H₂O:MeCN 1:1) and the concentration was measured using the UV absorbance at the λₘₐₓ, assuming the extinction coefficient of 10,000 M⁻¹ cm⁻¹ for all tryptathionine-containing peptides, with the exception of α-amanitin that has an extinction coefficient of 13,500 M⁻¹ cm⁻¹. Typically, 5.4-65.1 nmol (5-60 μg) was obtained and accurately quantified by UV-Vis spectroscopy using a 0.5 mL cuvette. For illustrative purposes, a quantity of 5.4 nmol of α-amanitin in a volume of 0.5 mL gives a concentration of 10.8 μM and an absorbance reading of 0.136 AU, a value that is fully within the ideal range for quantitative ultraviolet-visible (UV-Vis) spectroscopy.

(e) Sample preparation for cell toxicity assays: Following quantification, solutions with toxic peptides were re-lyophilized and re-suspended in a given volume of DMSO or H₂O to provide a 1 mM solution, which was then used in cell toxicity assays.

(f) Cell culture: Cells were cultured in Minimum Essential Medium Eagle—Alpha Modification (α-MEM) or high-sucrose Dulbecco's Modified Eagle Medium (DMEM), purchased from Gibco. Fetal bovine serum (FBS), 0.25% trypsin (with 1.3 mM ethylenediaminetetraacetic acid, EDTA), 0.85% Trypan blue, and the antibiotic mixture Pen/Strep (10K U/mL penicillin, 10K mg/mL streptomycin) were also purchased from Gibco. All cell culture plastic ware was obtained from Coming or Falcon. Cells were cultured at 37° C. in a humidified chamber with 5% CO₂. When used in cell culture, DMSO was purified by filtration through a 0.2 mm filter. All experiments were carried out in a laminar flow culture cabinet, unless otherwise noted. Absorbance measurements of the 96-well plates were obtained using a Beckman-Coulter DTX 880 multimode detector, equipped with an excitation filter of 595 nm.

Immortalized CHO cells had been stored in liquid nitrogen. To revive cells, a 1-mL tube of the frozen cells in medium containing 10% DMSO was warmed in a 30° C. water bath and diluted with 9 mL of fresh media. Media contained 10% FBS and 100 U/mL penicillin and 100 mg/mL streptomycin, unless otherwise indicated. The cells were incubated in a T-25 flask at 37° C. at 5% CO₂. After 24 hours, the medium was aspirated and replaced with fresh medium. When cells reached a level of 90-100% confluence, they were sub-cultured. The medium was removed, and the cells were treated with 0.25% trypsin containing 1.3 mM EDTA in the incubator. Once the cells were detached from the tissue culture flask, 3-5 mL media was added to quench the trypsin and the mixture was transferred to a 10 mL centrifuge tube. The mixture was centrifuged for 5 min at 8000 rpm, and the supernatant was discarded. The cells were suspended in fresh medium, diluted as required, and transferred to a new culture flask.

To assay cell viability, a nearly confluent tissue culture flask was trypsinized, and the cells were counted following treatment with Trypan blue, using a hemocytometer. The cells were then diluted to the appropriate stock concentrations in fresh medium and transferred in 100 μL to a 96-well plate using a multi-channel pipette. The number of cells plated varied from experiment to experiment. These were incubated at 37° C. and 5% CO₂ for a 24-hour period to allow for adherence. The medium was aspirated, and fresh medium was added, which contained the desired additives in DMSO. The cells were then re-incubated for 72 h. At the completion of the experiment, a 100 μL aliquot of 25 mg/mL MTT in phosphate-buffered saline (PBS) was added to each well. The plate was incubated three hours further to allow for the formation of the formazan product in viable cells. The media was carefully aspirated, and the purple product was solubilized in DMSO. The absorbance of each well was recorded at 595 nm. Data was processed in Microsoft Excel and GraphPad Prism. Experiments were performed in triplicates unless otherwise noted, and the error bars were calculated as the standard error of the mean.

Trypsinized cells were diluted to a concentration of 3.3×10⁴ cells/mL for CHO cells. Each cell line was plated in a 96-well plate, with 100 μL of the stock solution per well (5000 cells per well) and incubated for 24 hours. Stocks of α-amanitin or analogs were prepared at various concentrations, containing a maximum of 0.8% DMSO, and added to various wells, according to the desired final concentration. The cells were incubated for 72 hours, at which point viability was assessed as described.

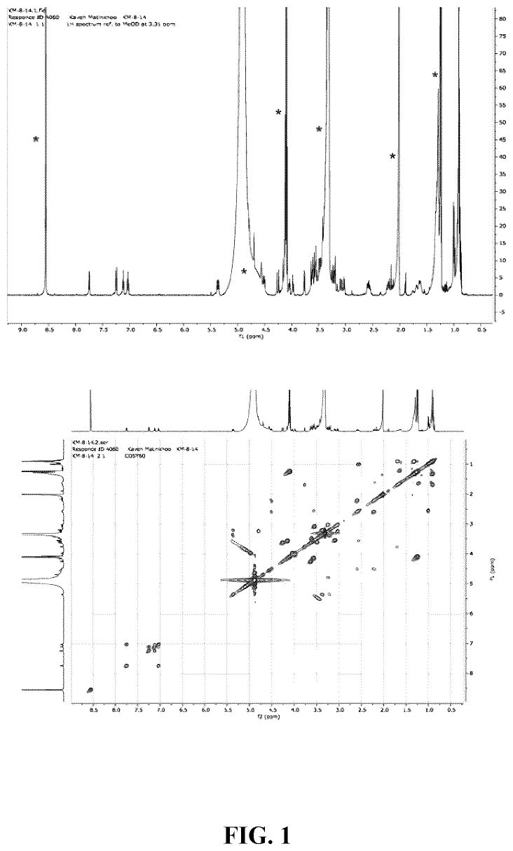

(g) NMR Spectra of the amanitins containing different Hyp analogs: FIGS. 1-8 contain the ¹H-NMR and COSY45 spectra (600 MHz, CD₃OD as NMR solvent) of the amanitins containing the Hyp analogs. Due to the small quantity of the synthetic analogs (100-500 nmol), the spectra provided are for comparison and demonstrating the purity of each compound. To prepare the samples for the NMR spectra, following the HPLC purification of each analog, pure compounds (100-300 nmol) were lyophilized from 0.1% formic acid in H₂O/acetonitrile (ACN; varying compositions) at least twice. The dry residue was re-suspended in CD₃OD (dried over 3 Å molecular sieves) and subjected to NMR studies on Bruker Avance 600 (with cryoprobe) or Bruker Avance 400inv spectrometers. The peaks resulting from sample preparation (e.g. formic acid, H₂O, MeOH) and solvent stabilizers are marked with an asterisk (*). Samples that had a quantity lower than 150 nmol were placed in a MeOD-Shigemi tube for NMR characterization.

(h) In vitro transcription assay of synthetic amanitins containing analogs of Hyp: Primers were purchased from IDT via standard phosphoramidite chemistry. dNTP and rNTP were purchased from Thermofisher. GoTaq polymerase and HeLaScribe*Nuclear Extract in vitro Transcription System was purchased from Promega. Taq polymerase was purchased from NEB. [³²P] α-dGTP (3000 Ci/mmol 10 mCi/ml EasyTide) and [³²P] α-GTP (3000 Ci/mmol 10 mCi/ml EasyTide) were purchased from Perkin Elmer. QIAquick PCR purifcation kit was purchased from Qiagen. AcGFP1-N1 was a gift (Addgene plasmid #54705; http://n2t.net/addgene:54705; RRID:Addgene_54705). DNA Primer sequences were:

P1 AcGFP1.FOR.V4
CAGTCGACGGTACCGC;
P2 AcGFP1.REV.V4
GCCCTCGAACTTCACCTC;
P3 AcGFP1.FOR.V2
CGCGGGCCCGGGATCCAC;
P4 AcGFP.REV.V2
ACCTCGGCGCGCGACTTGT.

The DNA template for transcription runoff was synthesized by PCR with template pAcGFP-N1, and primers P1 and P2. To a final volume of 20 μL, 1 x GoTaq Buffer, 250 μM of each dNTP, 500 nM of each primers P1 and P2, 10 μg/μL pAcGFP-N₁ and 0.05 U/μL GoTaq were employed to thermocycle for 30 cycles (30 s at 95° C., 30 s at 50° C. and 60 s at 72° C., Bio-Rad). A 1 μL aliquot of the amplified solution was resolved with 6×DNA loading dye in a 1% agarose gel containing 1% ethidium bromide and then visualized using GelDoc XR imager (Bio-Rad). Purification was completed via QIAquick PCR purification kit (Qiagen). The radioactive chromatographic standard was synthesized by PCR with template pAcGFP-N₁, and primers P3 and P4. To a final volume of 50 μL, 1 x Thermopol Buffer, 200 μM of each dNTP, 500 nM of each primers P3 and P4, 10 pg/μL pAcGFP-N₁ and 0.02 U/μL Vent polymerase, 1 μL [³²P] α-dGTP were employed to thermocycle for 30 cycles (30 s at 95° C., 30 s at 62° C. and 30 s at 72° C., Bio-Rad). A 1 μL aliquot of the amplified solution was resolved with 6×DNA loading dye in 8% denaturing PAGE and then visualized by autoradiography via the Typhoon 9200 imager (Molecular namics-Amersham-GE). Purification was completed via QIAquick PCR purification kit (Qiagen). The transcription runoff assay was modified from HeLaScribe® Nuclear Extract in vitro Transcription System (Promega). A 1.35× master mixture was formulated with 1.35× HeLa Nuclear Extract Transcription Buffer, 4.05 mM MgCl2, 540 μM rATP, 540 μM rCTP, 540 μM rUTP and 21.6 μM rGTP, 5.4 ng/μL DNA template and 3 μL [³²P] α-GTP. To a final volume of 8 μL, 1 X master mixture, 0-3 μM aqueous solutions containing α-amanitin or amanitin analogs (B-H), 0.32 U/μL HeLaScribe*Nuclear Extract were combined to transcribe for 60 min. The reaction was quenched by 56 μL HeLa Extract Stop Solution, followed by phenol-chloroform extraction and EtOH precipitation. The pellet was resolved by 8% denaturing PAGE and then visualized by autoradiography via the Typhoon 9200 imager (Molecular dynamics-Amersham-GE). Dosimetry was calculated by ImageJ, and the 3 parameter logistic fit was completed by Origin 2019 (OriginLab). For inhibitor I that demonstrates transcription activity E with half inhibitory concentration IC₅₀,

$$E = \frac{{IC}_{50}}{{IC}_{50} + [ I ]}$$

This assumes symmetry around IC₅₀ (asymmetry factor S=1) and no cooperativity (Hill's slope H=−1). All data were presented in mean±standard deviation (n=3).

(i) Cell viability assays (HEK293 and HEK293-OATP1B3 cells): To assess the effects of the synthetic amanitins containing derivatives of Hyp on normal and on OATP1B3 overexpressing cells, wildtype HEK293 (ATCC, Manassas, VA) and HEK293-OATP1B3 cells (HDPR)²⁷ were incubated with α-amanitin or synthetic analogs, and cytotoxicity was determined by a BrdU assay. HEK293 and HEK-OATP1B3 cells were plated at 2.5×10³ cells/well in a 1:1 mixture of Ham's F12 with DMEM containing 10% charcoal-stripped fetal calf serum (FCS) onto poly-D-lysine-coated 96-well plates and grown for 24 hours. Subsequently, cells were incubated with amanitin analogs at 8 different concentrations (1×10⁻⁶ M to 1.28×10⁻¹¹ M, serial 1:5 dilutions). Following 96 h of drug exposure, cell viability was determined by a BrdU incorporation assay (Cell Proliferation ELISA, BrdU, Roche) and chemiluminescence was measured using a FLUOstar Optima plate reader (BMG LABtech). Data analysis was performed with GraphPad Prism 8 (GraphPad Software, Inc., La Jolla, CA) software to plot curve fits.

### II. Synthetic Procedures

### (a) tert-Butyl-(1S,4S)-3-oxo-2-oxa-5-azabicyclo[2.2.1]heptane-5-carboxylate (11)

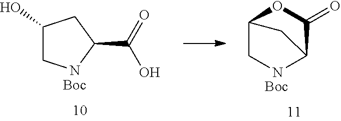

To a stirred ice-cold solution of Boc-Hyp-OH (10) (1.52 g, 6.58 mmol) and triphenylphosphine (PPh₃; 2.12 g, 7.89 mmol) in dry tetrahydrofuran (THF; 50 mL) under argon was added diisopropyl azodicarboxylate (DIAD, 1.54 mL, 7.89 mmol) dropwise at 0° C. The reaction mixture was allowed to warm up to room temperature and stirring was continued for 20 hours. The solvent was evaporated under reduced pressure, and the residue was directly purified by flash column chromatography using silica gel (ethyl acetate (EtOAc)/hex 30:70) to yield the product as a white solid (1.19 g, 85%). TLC (EtOAc:hex 60:40 v/v): R_{f}=0.35. ¹H NMR (300 MHz, Methylene Chloride-d₂) δ 5.05 (s, 1H), 4.50 (s, 1H), 3.50 (dd, J=9.9, 0.8 Hz, 1H), 3.39 (d, J=11.0 Hz, 1H), 2.23-2.13 (m, 1H), 1.98 (d, J=9.7 Hz, 1H), 1.45 (s, 9H). ¹³C NMR (75 MHz, Methylene Chloride-d₂) δ 171.60, 154.32, 81.43, 79.19, 50.39, 39.54, 28.54. HRMS ESI (m/z) calculated for C₁₀H₁₆NO₄ [M+H]⁺ 214.1079; found 214.1085.

### (b) 1-(tert-Butyl)-2-methyl (2S,4S)-4-hydroxypyrrolidine-1,2-dicarboxylate, cis-Boc-Hyp-OMe (12)

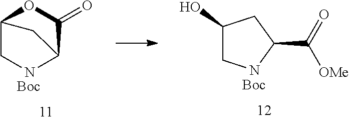

A solution of starting material (11) (570 mg, 2.69 mmol) and NaN₃ (352 mg, 5.38 mmol) in dry MeOH (80 mL) was stirred at 40° C. for 16 hours under argon. The solvent was evaporated under reduced pressure, and the residue was partitioned between H₂O and EtOAc. The aqueous layer was extracted with EtOAc (3×30 mL). The combined organic layers were washed with brine, dried over anhydrous Na₂SO₄, filtered and concentrated under reduced pressure. The residue was purified by flash column chromatography using silica gel (EtOAc/hex 30:70) to yield the product as a white solid (493 mg, 75%). TLC (EtOAc:hex 80:20 v/v): R_{f}=0.43. ¹H NMR (300 MHz, Methylene Chloride-d₂) δ 4.37-4.22 (m, 2H), 3.75 (s, 3H), 3.62-3.41 (m, 2H), 2.32 (tdd, J=13.8, 9.9, 4.7 Hz, 1H), 2.11-1.96 (m, 1H), 1.44 (s, 4H), 1.39 (s, 5H). ¹³C NMR (75 MHz, Methylene Chloride-d₂) δ 175.99, 154.89, 154.08, 80.56, 71.72, 70.74, 58.47, 58.25, 56.42, 55.93, 53.05, 52.93, 39.11, 38.33, 28.62, 28.52. HRMS ESI (m/z) calculated for C₁₁H₁₉NO₅Na [M+Na]⁺ 268.1161; found 268.1165.

### (c) 1-(tert-Butyl)-2-methyl-(2S,4S)-4-((methylsulfonyl)oxy)pyrolidine-1,2-dicarboxylate, cis-Boc-OMs-Pro-OMe (13)

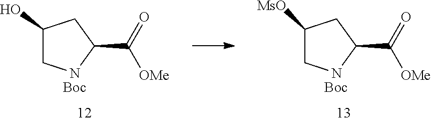 [SMILES: COC(=O)[C@@H]1C[C@H](O)CN1 ; COC(=O)[C@@H]1C[C@H](OS(C)(=O)=O)CN1]

A solution of 12 (1.5 g, 6.17 mmol) in dry dichloromethane (DCM, 45 mL) was cooled to 0° C. Triethylamine (1.22 mL, 8.76 mmol) and methanesulfonyl chloride (MsCl; 0.78 mL, 9.88 mmol) were added. The reaction was stirred at 0° C. overnight. Upon completion, the reaction mixture was successively washed with 0.1 M aq. HCl (40 mL), saturated aq. NaHCO₃ (40 mL) and brine. The organic layer was dried over anhydrous Na₂SO₄, filtered and concentrated under reduced pressure. The crude residue was purified by flash column chromatography using silica gel (EtOAc/hex 30:70 to 40:60, gradient) to yield the product as a colorless oil (1.87 g, 95%). TLC (EtOAc:hex 80:20 v/v): R_{f}=0.46. ¹H NMR (300 MHz, Chloroform-d) δ 5.34-5.06 (m, 1H), 4.60-4.32 (m, 1H), 3.87-3.63 (m, 5H), 3.00 (s, 3H), 2.60-2.38 (m, 2H), 1.46 (s, 4H), 1.41 (s, 5H). ¹³C NMR (75 MHz, Methylene Chloride-d₂) δ 172.50, 172.26, 154.19, 153.81, 80.87, 80.81, 79.45, 78.41, 57.90, 57.60, 53.04, 52.68, 52.57, 39.20, 37.60, 36.70, 32.30, 28.57, 28.45. HRMS ESI (m/z) calculated for C₁₂H₂₁NO₇SNa[M+Na]⁺ 346.0936; found 346.0934.

### (d) 1-(tert-Butyl)-2-methyl-(2S,4R)-4-azidopyrrolidine-1,2-dicarboxylate, trans-Boc-N₃-Pro-OMe (14)

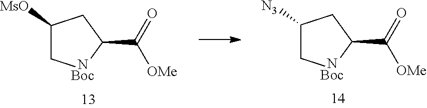 [SMILES: COC(=O)[C@@H]1C[C@H](OS(C)(=O)=O)CN1C(=O)OC(C)(C)C ; COC(=O)[C@@H]1C[C@@H](N=[N+]=[N-])CN1C(=O)OC(C)(C)C]

A solution of 13 (3 g, 9.28 mmol) and NaN₃ (1.23 g, 18.6 mmol) in dry DMSO (45 mL) was stirred at 80° C. for 4 hours. EtOAc (40 mL) and H₂O (40 mL) were then added to the reaction mixture. The aqueous phase was extracted with EtOAc (3×30 mL). The combined organic layers were washed with brine, dried over anhydrous Na₂SO₄, filtered and concentrated under reduced pressure. The crude residue was purified by flash column chromatography using silica gel (EtOAc/hex 40:60) to yield the product as a colorless oil (1.98 g, 79%). TLC (EtOAc:hex 80:20 v/v): R_{f}=0.62. ¹H NMR (300 MHz, Methylene Chloride-d) δ 4.40-4.26 (m, 1H), 4.24-4.13 (m, 1H), 3.71 (s, 3H), 3.69-3.59 (m, 1H), 3.57-3.41 (m, 1H), 2.41-2.24 (m, 1H), 2.22-2.08 (m, 1H), 1.44 (s, 4H), 1.38 (s, 5H). ¹³C NMR (75 MHz, Methylene Chloride-d₂) δ 173.48, 173.21, 154.42, 153.76, 80.86, 80.81, 60.00, 59.45, 58.29, 58.00, 52.70, 52.63, 51.98, 51.76, 36.78, 35.87, 28.58, 28.45. HRMS ESI (m/z) calculated for C₁₁H₁₈N₄O₄Na[M+Na]⁺ 293.1226; found 293.1222.

### (e) 1-(tert-Butyl)-2-methyl-(2S,4R)-4-cyanopyrrolidine-1,2-dicarboxylate, trans-Boc-CN-Pro-OMe (15)

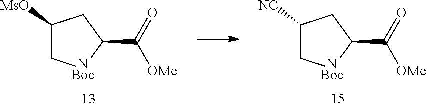 [SMILES: COC(=O)[C@@H]1C[C@H](OS(C)(=O)=O)CN1C(=O)OC(C)(C)C ; COC(=O)[C@@H]1C[C@@H](C#N)CN1C(=O)OC(C)(C)C]

To a stirred solution of 13 (420 mg, 1.3 mmol) in dry DMSO (7 mL) was added KCN (130 mg, 1.95 mmol). The resulting solution was heated to 80° C. for 4 hours. After addition of brine (5 mL) and H₂O (5 mL), the mixture was extracted with diethyl ether (4×10 mL). The combined organic layers were washed with brine, dried over anhydrous Na₂SO₄, filtered and concentrated under reduced pressure. The crude residue was purified by flash column chromatography using silica gel (EtOAc/hex 20:80 to 30:70, gradient) to yield the product as a colorless oil (99 mg, 30%). TLC (EtOAc:hex 80:20 v/v): R_{f}=0.63. ¹H NMR (300 MHz, Methylene Chloride-d₂) δ 4.50-4.29 (m, 1H), 3.94-3.78 (m, 1H), 3.72 (s, 3H), 3.67-3.56 (m, 1H), 3.34-3.16 (m, 1H), 2.61-2.41 (m, 1H), 2.40-2.26 (m, 1H), 1.44 (s, 3H), 1.38 (s, 6H). ¹³C NMR (75 MHz, Methylene Chloride-d₂) δ 172.89, 172.69, 153.92, 153.39, 119.77, 119.72, 81.29, 81.24, 58.48, 58.30, 52.89, 52.84, 49.83, 49.69, 35.13, 34.19, 28.53, 28.42, 27.67, 27.03. HRMS ESI (m/z) calculated for C₁₂H₁₈N₂O₄Na[M+Na]⁺ 277.1164; found 277.1161.

### (f) 1-(tert-Butyl)-2-methyl-(2S,4R)-4-(acetylthio)pyrrolidine-1,2-dicarboxylate, trans-Boc-SAc-Pro-OMe (16)

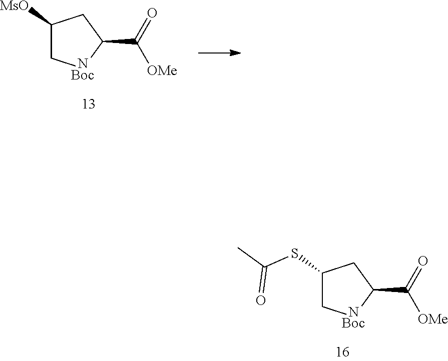 [SMILES: COC(=O)[C@@H]1C[C@H](OS(C)(=O)=O)CN1C(=O)OC(C)(C)C ; COC(=O)[C@@H]1C[C@@H](SC(C)=O)CN1C(=O)OC(C)(C)C]

A solution of the mesylated Hyp (13) (4 g, 12.4 mmol) and freshly prepared potassium thioacetate (1.84 g, 16.1 mmol) in dry DMF (50 mL) was stirred at 70° C. under argon for 4 hours. The reaction mixture was diluted with EtOAc (100 mL), the pH was adjusted to 2 by addition of 1 M aq. HCl, and the resulting mixture was washed with ice-cooled brine (2×75 mL). The organic layer was dried over anhydrous Na₂SO₄, filtered and concentrated under reduced pressure. The crude residue was purified by flash column chromatography using silica gel (EtOAc/hex 7:93 to 10:90 to 13:87, gradient) to afford the product as a red oil (1.95 g, 52%). TLC (EtOAc:hex 30:70 v/v): R_{f}=0.33. ¹H NMR (300 MHz, Methylene Chloride-d₂) δ 4.30 (ddd, J=16.0, 8.4, 4.7 Hz, 1H), 4.00 (p, J=6.7 Hz, 1H), 3.93-3.83 (m, 1H), 3.72 (s, 3H), 3.32 (ddd, J=17.4, 11.0, 6.1 Hz, 1H), 2.42-2.32 (m, 1H), 2.31 (s, 3H), 2.28-2.12 (m, 1H), 1.43 (s, 4H), 1.37 (s, 5H). ¹³C NMR (75 MHz, Methylene Chloride-d₂) δ 195.16, 173.43, 173.15, 154.35, 153.69, 80.64, 58.96, 58.69, 52.69, 52.63, 52.50, 52.15, 40.21, 39.88, 37.18, 36.06, 30.96, 28.60, 28.47. HRMS ESI (m/z) calculated for C₁₃H₂₁NO₅SNa[M+Na]⁺ 326.1038; found 326.1034.

### (g) (2S,4R)-1-(((9H-fluoren-9-yl)methoxy)carbonyl)-4-azidopyrrolidine-2-carboxylic acid, trans-Fmoc-N3-Pro-OH (21)

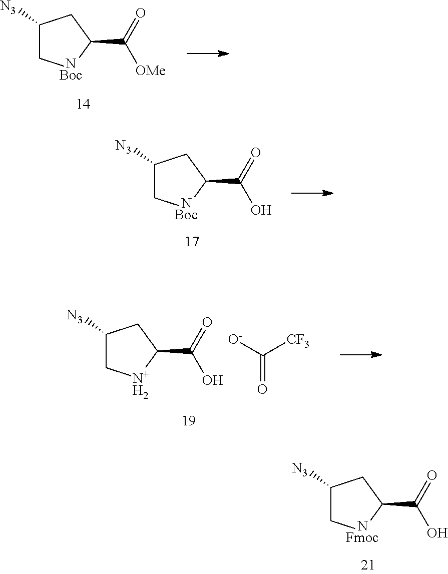

To a mixture of 14 (3.79 g, 14.03 mmol) in THF (125 mL) at 0° C. was added a 5% aqueous solution of LiOH (125 mL). The reaction mixture was stirred at 0° C. overnight, followed by removal of THF under reduced pressure. The remaining aqueous phase was washed with EtOAc (3×50 mL), acidified to a pH of about 2 with 1 M aq. HCl, and extracted with EtOAc (3×100 mL). The combined organic layers were washed with brine, dried over anhydrous Na₂SO₄, filtered and concentrated to yield the free acid (17) as a colorless oil. Trifluoroacetic acid (TFA, 45 mL) was added to a solution of the crude 17 in DCM (125 mL). After stirring at room temperature for 30 min, the reaction mixture was concentrated under reduced pressure. The residual TFA was co-evaporated with diethyl ether (2×50 mL) and toluene (2×30 mL) to yield the crude Boc-deprotected product (19) as a light brown solid. 19 was resuspended in 1,4-dioxane/H₂O (70 mL:30 mL) and NaHCO₃ (1.69 g, 28.06 mmol) was added at once. The reaction was stirred at room temperature for 10 min, followed by the addition of 9-fluorenylmethyl N-succinimidyl carbonate (Fmoc-Osu; 5.2 g, 15.43 mmol). After 3 hours, the reaction mixture was acidified to a pH of about 1 with 1 M aq. HCl, and was extracted with EtOAc (4×75 mL). The combined organic layers were washed with brine, dried over anhydrous Na₂SO₄, filtered and concentrated under reduced pressure. The crude residue was purified by flash column chromatography using silica gel (MeOH/DCM/(acetic acid (HOAc) 2:98:0.1 to 4:96:0.1, gradient). The product contained acetic acid from the column which was co-evaporated with DCM/heptane (2×20 mL) to yield the dry product as a white solid (3.67 g, 69% over 3 steps). TLC (MeOH:DCM:HOAc 10:90:1 v/v): R_{f}=0.43. ¹H NMR (300 MHz, Methylene Chloride-d₂) δ 9.56 (s, 1H), 7.84-7.69 (m, 2H), 7.66-7.50 (m, 2H), 7.47-7.23 (m, 4H), 4.52-4.15 (m, 5H), 3.75-3.52 (m, 2H), 2.48-2.18 (m, 2H). ¹³C NMR (75 MHz, Methylene Chloride-d₂) δ 177.03, 175.51, 156.04, 154.74, 144.48, 144.30, 144.25, 141.82, 128.32, 128.21, 127.65, 125.57, 125.46, 125.40, 120.53, 120.47, 68.68, 68.29, 59.84, 59.21, 58.47, 57.76, 52.35, 52.00, 47.67, 47.61, 36.88, 35.32. HRMS ESI (m/z) calculated for C₂₀H₁₉N₄O₄ [M+H]⁺ 379.1406; found 379.1402.

### (h) (2S,4R)-1-(((9H-fluoren-9-yl)methoxy)carbonyl)-4-cyanopyrrolidine-2-carboxylic acid, trans-Fmoc-CN-Pro-OH (22)

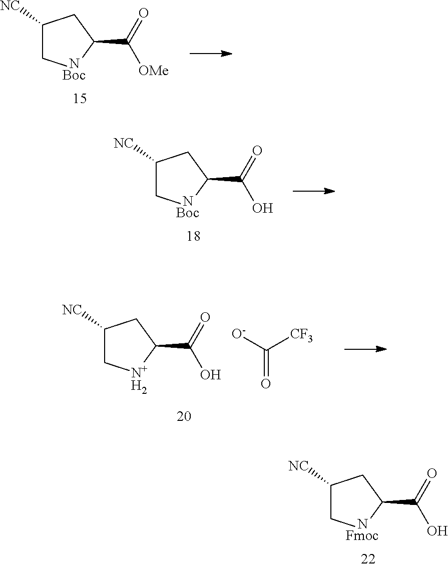

A procedure similar to that described above for trans-Fmoc-azido-Pro-OH (21) was followed. The product was a white solid. Yield over 3 steps: 32%. TLC (MeOH:DCM:HOAc 10:90:1 v/v): Rf=0.42. ¹H NMR (300 MHz, Methylene Chloride-d₂) δ 7.80 (d, J=7.5 Hz, 1.36H), *rotamer: δ 7.75 (d, J=7.5 Hz, 0.64H), 7.59 (d, J=7.3 Hz, 1.34H), *rotamer: 7.53 (d, J=6.3 Hz, 0.69H), 7.47-7.39 (m, 2H), 7.38-7.27 (m, 2H), 4.53 (dd, J=8.4, 2.9 Hz, 0.65H), *rotamer: 4.37 (dd, J=8.6, 2.9 Hz, 0.35H), 4.47 (d, J=6.8 Hz, 1H), 4.29 (t, J=6.7 Hz, 0.67H), *rotamer: 4.21-4.14 (m, 0.33H), 3.92-3.79 (m, 1H), 3.75-3.61 (m, 1H), 3.33-3.15 (m, 1H), 2.66-2.55 (m, 1H), 2.55-2.38 (m, 1H). ¹³C NMR (75 MHz, Methylene Chloride-d₂) δ 175.52, 173.71, 155.98, 154.15, 144.12, 141.86, 128.42, 128.28, 127.72, 125.49, 120.61, 119.29, 119.08, 69.03, 68.24, 58.85, 57.85, 50.11, 49.72, 47.58, 35.17, 33.33, 27.76, 26.89. HRMS ESI (m/z) calculated for C₂1H₁₈N₂O₄Na[M+Na]⁺ 385.1164; found 385.1172.

### (i) (2S,4R)-1-(((9H-fluoren-9-yl)methoxy)carbonyl)-4-((acetamidomethyl)thio) pyrrolidine-2-carboxylic acid, trans-Fmoc-SAcm-Pro-OH (26)

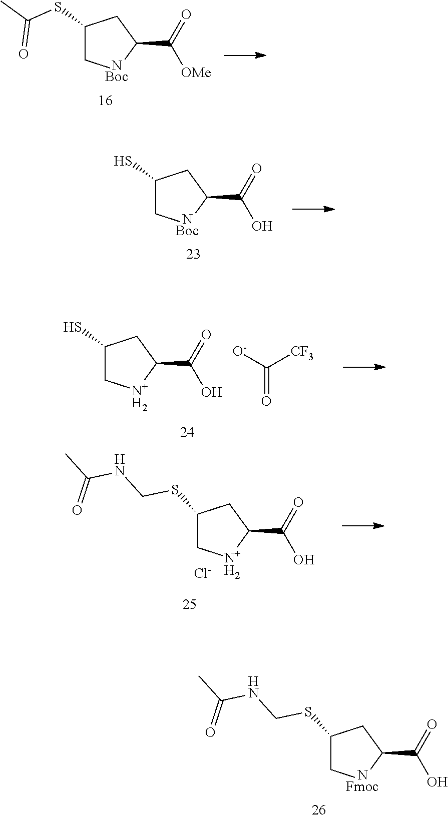

To a solution of 16 (2.48 g, 8.18 mmol) in THF (70 mL) was added a 5% aqueous solution of LiOH (70 mL) at 0° C. The reaction mixture was stirred at 0° C. overnight. The THF was removed under reduced pressure. The remaining aqueous phase was washed with EtOAc (2×20 mL), acidified to a pH of about 2 by adding 1M aq. HCl, extracted with EtOAc (5×20 mL), washed with brine, dried over anhydrous Na₂SO₄, filtered and concentrated under reduced pressure to obtain the crude trans-Boc-SH-Pro-OH (23). Crude 23 was re-dissolved in DCM (17 mL), and TFA (9 mL) was added to the mixture. The reaction was then stirred at room temperature for 30 min, and the solvent and TFA were evaporated under reduced pressure. The residual TFA was co-evaporated with Et₂O, followed by toluene. The fully unprotected proline obtained from the last reaction (24) was suspended in H₂O (5 mL). To the resulting mixture was added 12M HCl (0.63 mL) at 0° C., followed by the addition of (N-hydroxymethyl)acetamide to provide the acetamidomethyl (Acm) group (0.87 g, 9.74 mmol). The reaction mixture was warmed up to room temperature and stirred for 48-72 hours, at which point it was washed with EtOAc (2×15 mL). The remaining aqueous layer was evaporated under reduced pressure to yield the crude trans-SAcm-Pro-OH (25). The crude product (25) was resuspended in 1,4-dioxane/H₂O (41 mL:17.5 mL) and NaHCO₃ (1.52 g, 18 mmol) was added at once. The reaction was stirred at room temperature for 10 min, followed by the addition of Fmoc-OSu (2.16 g, 9.0 mmol). After 3 hours, the reaction mixture was acidified to a pH of about 1 with 1 M aq. HCl. The resulting mixture was then extracted with EtOAc (4×10 mL). The combined organic layers were washed with brine, dried over anhydrous Na₂SO₄, filtered then concentrated under reduced pressure. The crude residue was purified by flash column chromatography using silica gel (MeOH/DCM/HOAc 2:98:0.1 to 4:96:0.1, gradient). The product contained acetic acid from the column which was co-evaporated with DCM/heptane (2×20 mL) to yield the dry product as a white solid (900 mg, 25% over 4 steps). TLC (MeOH:DCM:HOAc 10:90:1 v/v/v): R_{f}=0.22 ¹H NMR (300 MHz, Methanol-d₄) δ 7.73 (dd, J=7.5, 4.5 Hz, 2H), 7.61-7.51 (m, 2H), 7.38-7.21 (m, 4H), 4.41-4.23 (m, 5H), 4.15 (dt, J=22.3, 6.9 Hz, 1H), 3.85 (dt, J=10.8, 7.1 Hz, 1H), 3.53 (h, J=6.7 Hz, 1H), 3.34 (td, J=10.9, 5.5 Hz, 1H), 2.45-2.28 (m, 1H), 2.28-2.12 (m, 1H), 1.91 (s, 1.5H), 1.91 (s, 1.5H). ¹³C NMR (75 MHz, Methanol-d4) δ 175.60, 175.38, 173.10, 156.37, 156.22, 145.26, 145.03, 142.57, 128.83, 128.20, 126.19, 126.12, 120.96, 69.20, 68.80, 60.03, 59.86, 54.43, 53.98, 41.89, 41.66, 41.13, 38.86, 37.82, 22.62. HRMS ESI (m/z) calculated for C₂₃H₂₄N₂O₅SNa[M+Na]⁺ 463.1304; found 463.1303.

### (j) Synthesis of (N₃-Pro)-hexapeptide (27a)

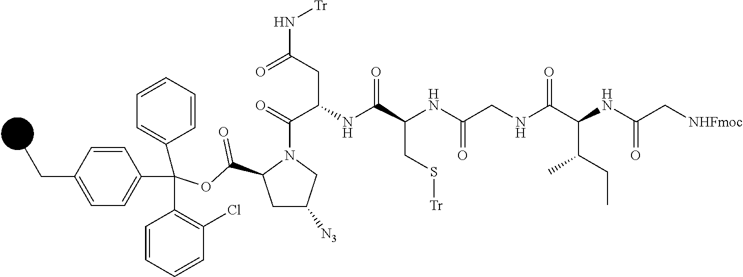

trans-Fmoc-N₃-Pro-OH was loaded on a 2-chlorotrityl chloride (CTC) resin according to the following protocol. To a flame-dried flask was added CTC resin (1.1 g, 1.2 mmol/g, 200-400 mesh), which was then suspended in dry CH₂Cl₂ (9 mL). To this flask was added trans-Fmoc-N₃-Pro-OH (1.13 g, 3 mmol) and N,N-diisopropylethylamine (DIPEA; 1.3 mL, 7.5 mmol). The reaction was stirred under argon at room temperature overnight and transferred to a spin column. The resin was washed three times with DMF and DCM (8 mL for each wash). Unreacted sites of the resin were capped by applying a solution of CH₂Cl₂:MeOH:DIPEA (8 mL of an 80:15:5 mixture, 20 min), and then washed with CH₂Cl₂ (3×8 mL), then DMF (3×8 mL), then CH₂Cl₂ (3×8 mL) again. The resin was dried in vacuo with P₂O₅ to remove residual solvent. Resin loading was determined using manufacturer's protocols. Briefly, a weighed amount of resin (2-3 mg) was treated with a 2% solution of DBU in DMF for 30 minutes. The solution was diluted and the UV absorbance of the liberated dibenzofulvene was measured at 304 nm, with an absorption coefficient of ε₃₀₄=7624 M⁻¹ cm⁻¹. Measured resin loading: 0.9 mmol/g. Five equivalents of the following N^{α}-Fmoc-amino acids (Asn(NTr), Cys(STr), Gly, Ile, Gly) and five equivalents of coupling agent (benzotriazol-1-yl-oxytripyrrolidinophosphonium hexafluorophosphate (PyBOP) or 2-(1H-benzotriazol-1-yl)-1,1,3,3-tetramethyluronium hexafluorophosphate (HBTU)/hydroxybenzotriazole (HOBt).H₂O) in DMF were applied sequentially to the growing N-terminus. In general, the following protocol was followed for coupling: Resin was placed in a Zeba spin column (up to 400 mg in a 5 mL column or 1 g in a 10 mL column) and pre-swollen in DMF (8 mL) for 30 min (3×8 mL, 10 min each, draining DMF after each swelling). The solvent was drained and the N-terminal Fmoc protecting group was removed by shaking with 20% piperidine in DMF (8 mL for 5 min, 8 mL for 10 min). Following deprotection, the resin was washed with DMF (3×8 mL), followed by CH₂Cl₂ (3×8 mL) and again with DMF (3×8 mL). The second amino acid in the linear sequence, Fmoc-Asn(Trt)-OH, was coupled to the first amino acid on the resin (Hyp analog) using PyBOP (5 eq.), DIPEA (10 eq.) in DMF (8 mL). The next amino acids were coupled to the resin using the suitably protected amino acid (Fmoc-Xaa(R)-OH, 5 eq.), coupling agent HBTU (5 eq.), HOBt.H₂O (5 eq.) and DIPEA (10 eq.) in DMF (8 mL). The reactions were slowly shaken on a vortexer at minimum speed for 2 h. For procedures in which a non-commercially available amino acid was used, fewer equivalents and longer coupling times were employed. Double coupling was performed when the free N-terminus on the resin was derived from a hydroxyproline residue or asparagine. Often, a Kaiser test was performed to check for complete couplings. Alternatively, a small amount of resin was removed from the batch and was deprotected with 25% hexafluoroisopropanol (HFIP) in CH₂Cl₂, and the released peptide was analyzed by LRMS-ESI. When the reaction was complete, the coupling mixture was drained, and washed with DMF (3×8 mL).

### (k) Synthesis of (CN-Pro)-hexapeptide (28a)

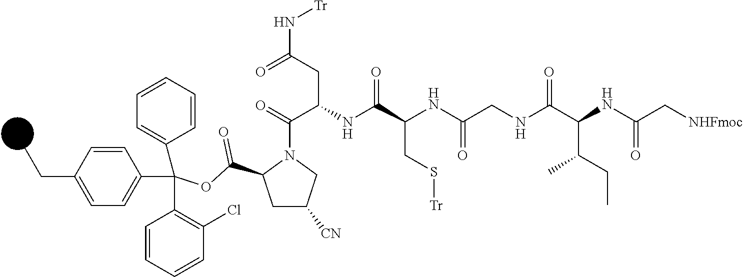

A procedure similar to the synthesis of (N₃-Pro)-hexapeptide (27a) was employed. To summarize, trans-Fmoc-CN-Pro-OH (650 mg, 1.8 mmol), DIPEA (0.78 mL, 4.5 mmol), CTC resin (660 mg) and dry DCM (5 mL) were stirred together overnight at room temperature. The Fmoc loading of the resin after the coupling of the first amino acid was measured to be 0.84 mmol/g. The subsequent amino acids were coupled to trans-CN-Pro-OH according to the procedure for 27a.

### (l) Synthesis of (SAcm-Pro)-hexapeptide (29a)

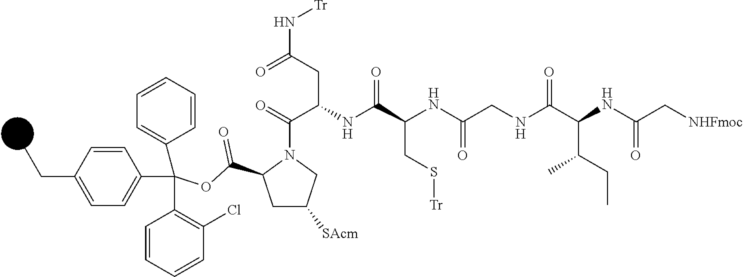

A procedure similar to the synthesis of (N₃-Pro)-hexapeptide (27a) was employed. To summarize, trans-Fmoc-SAcm-Pro-OH (500 mg, 1.13 mmol), DIPEA (0.49 mL, 2.82 mmol), CTC resin (500 mg) and dry DCM (4 mL) were stirred together overnight at room temperature. The Fmoc loading of the resin after the coupling of the first amino acid was measured to be 0.91 mmol/g. The subsequent amino acids were coupled to trans-SAcm-Pro-OH according to the procedure for 27a.

### (m) (2S)-1-(tert-butoxycarbonyl)-3a-fluoro-1,2,3,3a,8,8a-hexahydropyrrolo[2,3-b]indole-2-carboxylic acid, Boc-Fpi-OH (30)

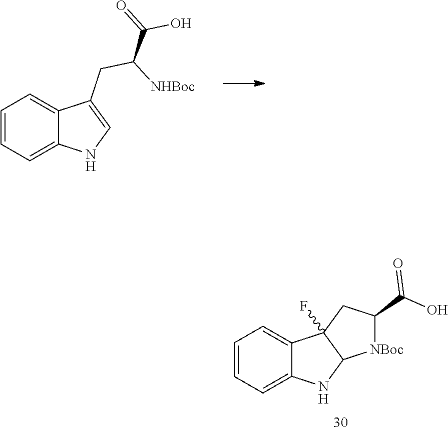 [SMILES: CC(C)(C)OC(=O)N[C@@H](Cc1c[nH]c2ccccc12)C(=O)O ; CC(C)(C)OC(=O)N1C2Nc3ccccc3C2(F)C[C@H]1C(=O)O]

N-Fluorocollidinium triflate (FP-T300, 5 g, 17.3 mmol) was added to a solution of Boc-Trp-OH (2.63 g, 8.65 mmol) in dry DCM (120 mL) in a dry round-bottom flask equipped with a condenser under argon. The resulting solution was stirred at 40° C. for 3 hours. Upon completion of the reaction (TLC), its contents were transferred to a separatory funnel and washed with 0.001 M aq. HCl (2×100 mL) and brine (100 mL). The organic layer was dried over anhydrous Na₂SO₄, filtered and concentrated under reduced pressure to yield the crude product as a light-brown foam, which was used in the next step without further purification. TLC (MeOH:DCM:HOAc 10:90:1 v/v): R_{f}=0.42.

### (n) Synthesis of (N₃-Pro)-heptapeptide (27c)

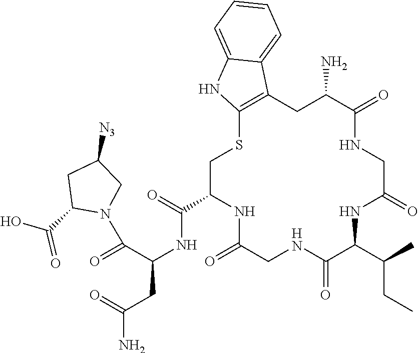 [SMILES: CC[C@H](C)[C@@H]1NC(=O)CNC(=O)[C@@H](N)Cc2c([nH]c3ccccc23)SC[C@@H](C(=O)N[C@@H](CC(N)=O)C(=O)N2C[C@H](N=[N+]=[N-])C[C@H]2C(=O)O)NC(=O)CNC1=O]

N^{α}-Boc-Fpi-OH (30) (crude, 1.56 g, 5 eq) was coupled to the N-terminus of the hexapeptide 27a (1.1 g of resin, 0.89 mmol/g loading) as described herein for amino acid couplings on CTC resin. After the coupling was completed (to yield the corresponding linear heptapeptide 27b), the resin was washed with DMF (3×8 mL), MeCN (3×8 mL), EtOAc/EtOH 1:1 (3×8 mL) and CH₂Cl₂ (3×8 mL). The resin-bound linear heptapeptide (27b) was transferred to a round-bottom flask and stirred in TFA/DCM 1:1 (30 mL) for 1 hour to induce the Savige-Fontana reaction and the global deprotection of acid-labile protecting groups. Triisopropyl silane (TIPS, 0.6 mL) and H₂O (0.6 mL) were added to the reaction and stirring was continued for 1 hour. The resin was filtered over glass wool and washed with CH₂Cl₂ (20 mL). The combined filtrate was evaporated under reduced pressure, followed by co-evaporation with Et₂O (2×15 mL), and then dried in vacuo. The residue was then re-dissolved in 0.1% FA (formic acid) in H₂O/MeCN 1:1 and purified by C₁₈ Sep Pak. Fractions containing the desired product were detected by analytical HPLC and mass spectrometry. These fractions were combined, lyophilized and re-suspended in a known volume of MeOH. Concentration and mass (180 mg) of the product (27c) were determined by its UV absorbance at 290 nm, with an extinction co-efficient of 10,000 M-^{l}cm⁻¹, that is lower than that of the natural product (13,500 M⁻¹ cm⁻¹). HPLC (gradient A): t_{R}=14.0 min; λₘₐₓ 290 nm. HRMS-ESI (m/z): [M+H]⁺ calcd. for C₃₃H₄₅N₁₂O₉S, 785.3148; found 785.3148. ¹H NMR (400 MHz, Methanol-d₄) δ 7.70-7.61 (m, 1H), 7.39-7.27 (m, 1H), 7.26-7.11 (m, 1H), 7.11-7.01 (m, 1H), 6.96-6.88 (m, 0.3H), 5.08-4.97 (m, 1H), 4.65-4.44 (m, 1H), 4.41-4.18 (m, 3H), 4.16-3.71 (m, 7H), 3.63-3.53 (m, 1H), 3.43 (dd, J=13.7, 4.7 Hz, 1H), 3.19 (ddd, J=35.0, 14.2, 7.6 Hz, 1H), 2.79-2.46 (m, 2H), 2.40-2.26 (m, 1H), 2.23-2.12 (m, 1H), 1.95-1.79 (m, 1H), 1.68-1.50 (m, 1H), 1.18 (dt, J=14.4, 8.4 Hz, 1H), 1.00-0.86 (m, 6H).

### (o) Synthesis of (CN-Pro)-heptapeptide (28c)

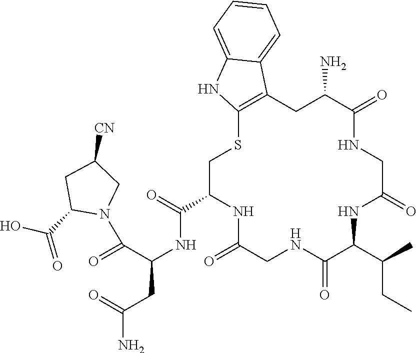 [SMILES: CC[C@H](C)[C@@H]1NC(=O)CNC(=O)[C@@H](N)Cc2c([nH]c3ccccc23)SC[C@@H](C(=O)N[C@@H](CC(N)=O)C(=O)N2C[C@H](C#N)C[C@H]2C(=O)O)NC(=O)CNC1=O]

N^{α}-Boc-Fpi-OH (30) (crude, 890 mg, 5 eq) was coupled to the N-terminus of the hexapeptide 28a (660 mg of resin, 0.84 mmol/g loading) as described herein for amino acid couplings on CTC resin. After the coupling was completed (to yield the corresponding linear heptapeptide 28b), the resin was washed with DMF (3×8 mL), MeCN (3×8 mL), EtOAc/EtOH 1:1 (3×8 mL) and CH₂Cl₂ (3×8 mL). The resin-bound linear heptapeptide (28b) was transferred to a round-bottom flask and stirred in TFA/DCM 1:1 (15 mL) for 1 hour to induce the Savige-Fontana reaction and the global deprotection of acid-labile protecting groups. Triisopropyl silane (TIPS, 0.3 mL) and H₂O (0.3 mL) were added to the reaction and stirring was continued for 1 hour. The resin was filtered over glass wool and washed with CH₂Cl₂ (20 mL). The combined filtrate was evaporated in vacuo, followed by co-evaporation with Et₂O (2×15 mL), and then dried under reduced pressure. The residue was then re-dissolved in 0.1% FA (formic acid) in H₂O/MeCN 1:1 and purified by C₁₈ Sep Pak. Fractions containing the desired product were detected by analytical HPLC and mass spectrometry. These fractions were combined, lyophilized and re-suspended in a known volume of MeOH. Concentration and mass (110 mg) of the product (28c) were determined by its UV absorbance at 290 nm, with an extinction co-efficient of 10,000 M⁻¹ cm⁻¹, that is lower than that of the natural product (13,500 M-^{l}cm⁻¹). HPLC (gradient A): t_{R}=7.5 min; λₘₐₓ 290 nm HRMS-ESI (m/z): [M+H]⁺ calcd. for C₃₄H₄₅N₁₀O₉S, 769.3086; found 769.3093. ¹H NMR (400 MHz, Methanol-d₄) δ 7.68-7.60 (m, 1H), 7.38-7.32 (m, 1H), 7.22-7.14 (m, 1H), 7.12-7.04 (m, 1H), 5.08-4.92 (m, 1H), 4.62-4.51 (m, 1H), 4.50-4.38 (m, 1H), 4.34-4.20 (m, 1H), 4.13-3.96 (m, 3H), 3.95-3.69 (m, 3H), 3.66-3.54 (m, 1H), 3.53-3.38 (m, 1H), 3.29-3.14 (m, 1H), 2.80-2.67 (m, 1H), 2.66-2.29 (m, 3H), 1.96-1.80 (m, 1H), 1.67-1.51 (m, 1H), 1.28-1.11 (m, 1H), 1.01-0.86 (m, 6H).

### (p) Synthesis of (SAcm-Pro)-heptapeptide (29c)

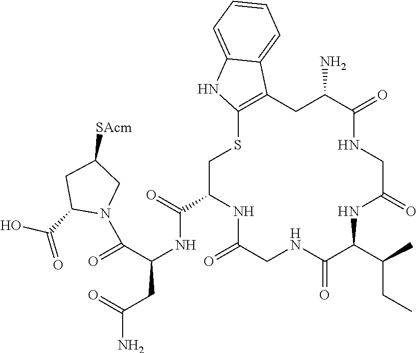

N^{α}-Boc-Fpi-OH (30) (crude, 720 mg, 5 eq) was coupled to the N-terminus of the hexapeptide 29a (500 mg of resin, 0.9 mmol/g loading) as described herein for amino acid couplings on CTC resin. After the coupling was completed (to yield the corresponding linear heptapeptide, 29b), the resin was washed with DMF (3×8 mL), MeCN (3×8 mL), EtOAc/EtOH 1:1 (3×8 mL) and CH₂Cl₂ (3×8 mL). The resin-bound linear heptapeptide (29b) was transferred to a round-bottom flask and stirred in TFA/DCM 1:1 (15 mL) for 1 hour to induce the Savige-Fontana reaction and the global deprotection of acid-labile protecting groups. Triisopropyl silane (TIPS, 0.3 mL) and H₂O (0.3 mL) were added to the reaction and stirring was continued for 1 hour. The resin was filtered over glass wool and washed with CH₂Cl₂ (20 mL). The combined filtrate was evaporated in vacuo, followed by co-evaporation with Et₂O (2×15 mL), and then dried under reduced pressure. The residue was then re-dissolved in 0.1% FA (formic acid) in H₂O/MeCN 1:1 and purified by C₁₈ Sep Pak. Fractions containing the desired product were detected by analytical HPLC and mass spectrometry. These fractions were combined, lyophilized and re-suspended in a known volume of MeOH. Concentration and mass (90 mg) of the product (29c) were determined by its UV absorbance at 290 nm, with an extinction co-efficient of 10,000 M⁻¹ cm⁻¹, that is lower than that of the natural product (13,500 M⁻¹ cm⁻¹). HPLC (gradient A): t_{R}=13.1 min; λmax 290 nm. HRMS-ESI (m/z): [M+H]⁺ calcd. for C₃₆H₅₁N₁₀O₁₀S₂, 847.3226; found 847.3229. ¹H NMR (400 MHz, Methanol-d₄) δ 7.67-7.59 (m, 1H), 7.38-7.28 (m, 1H), 7.26-7.11 (m, 1H), 7.11-7.02 (m, 1H), 6.96-6.88 (m, 0.4H), 5.07-4.98 (m, 1H), 4.70-4.47 (m, 2H), 4.45-4.27 (m, 3H), 4.27-4.17 (m, 1H), 4.15-3.82 (m, 5H), 3.82-3.36 (m, 5H), 3.29-3.11 (m, 1H), 2.91-2.44 (m, 3H), 2.39-2.26 (m, 1H), 2.20-2.05 (m, 1H), 1.95-1.76 (m, 1H), 1.68-1.49 (m, 1H), 1.30-1.10 (m, 1H), 1.00-0.86 (m, 6H).

### (q) Synthesis of (N₃-Pro)-monocyclic octapeptide (27d)

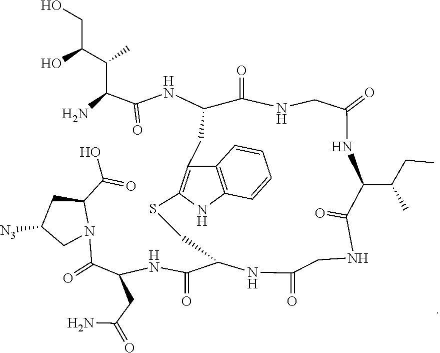 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](N=[N+]=[N-])C[C@H]2C(=O)O)NC(=O)CNC1=O]

(N₃-Pro)-heptapeptide 27c (20 mg, 25.5 μmol, 1 eq) was dissolved in 300 μL DMF. To this solution was added a solution of (2S,3R,4R)-O^{γ},O^{δ}-bis-TBS-N^{α}-Fmoc-dihydroxyisoleucine-OSu (prepared according to the synthesis in Matinkhoo et al., JACS, 2018, 140, 21, 6513-6517; 50 mg, 70.4 μmol, 2.8 eq) in 200 μL DMF, followed by 11 μL of DIPEA (final pH 8). The resulting mixture was set to stand at room temperature for 48 hours. After completion of the coupling (HPLC, MS), 20 μL of Et₂NH was added to the reaction mixture, and the reaction was stirred for another 2 hours. To the reaction mixture was added 500 μL of 1M tetra-n-butylammonium fluoride (TBAF) in THF and 6 μL of HOAc, and the resulting solution was set to stand at room temperature for 1 hour. The reaction was further acidified to a pH of about 3 with 1 M aq. HCl, and the solvent was removed in vacuo. The crude octapeptide was resuspended in 0.1% formic acid in H₂O/MeCN (1:4) and purified using C18 Sep Pak (isolated yield: 11 mg, 46.3% over 3 steps). HPLC (gradient D): t_{R}=20.0 min; λₘₐₓ 290 nm LRMS-ESI (m/z): [M−H]⁻ calcd. for C₃₉H₅₄N₁₃O₁₂S, 928.37; found 928.5.

### (r) Synthesis of (CN-Pro)-monocyclic octapeptide (28d)

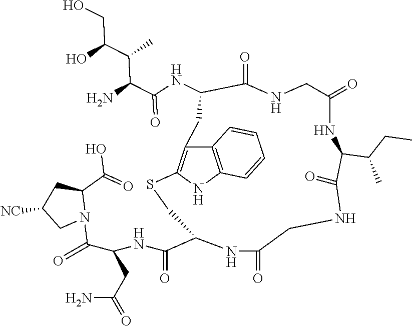 [SMILES: CC[C@H](C)[C@@H]1NC(=O)CNC(=O)[C@@H](NC(=O)[C@@H](N)[C@@H](C)[C@@H](O)CO)Cc2c([nH]c3ccccc23)SC[C@@H](C(=O)N[C@@H](CC(N)=O)C(=O)N2C[C@H](C#N)C[C@H]2C(=O)O)NC(=O)CNC1=O]

(CN-Pro)-heptapeptide 28c (9 mg, 11.7 μmol, 1 eq) was dissolved in 100 μL DMF. To this solution was added a solution of (2S,3R,4R)—O_{γ},O^{δ}-bis-TBS-N^{α}-Fmoc-dihydroxyisoleucine-OSu (prepared according to the synthesis in Matinkhoo et al., JACS, 2018, 140, 21, 6513-6517; 20 mg, 28.2 μmol, 2.4 eq) in 100 μL DMF, followed by 4 μL of DIPEA (final pH 8). The resulting mixture was let stand at room temperature for 48 hours. After completion of the coupling (HPLC, MS), 9 μL of Et₂NH was added to the reaction mixture, and the reaction was stirred for another 2 hours. To the reaction mixture was added 270 μL of 1M TBAF in THF and 2.4 μL of HOAc, and the resulting solution was let stand at room temperature for 1 hour. The reaction was further acidified to a pH of about 3 with 1 M aq. HCl, and the solvent was removed in vacuo. The crude octapeptide was resuspended in 0.1% formic acid in H₂O/MeCN (1:4) and purified using C₁₈ Sep Pak (isolated yield: 7.2 mg, 67.3% over 3 steps). HPLC (gradient B): t_{R}=16.8 min; max 290 nm LRMS-ESI (m/z): [M−H]⁻ calcd. for C₄₀H₅₄N₁₁O₁₂S, 912.37; found 912.6.

### (s) Synthesis of (SAcm-Pro)-monocyclic octapeptide (29d)

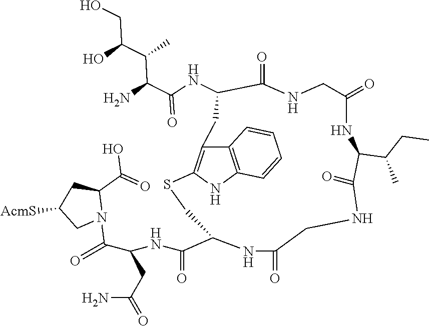

(SAcm-Pro)-heptapeptide 29c (8.7 mg, 10.3 μmol, 1 eq) was dissolved in 100 μL DMF. To this solution was added a solution of (2S,3R,4R)—O_{γ},O^{δ}-bis-TBS-N^{α}-Fmoc-dihydroxyisoleucine-OSu (prepared according to the synthesis in Matinkhoo et al., JACS, 2018, 140, 21, 6513-6517; 20 mg, 28.2 μmol, 2.7 eq) in 100 μL DMF, followed by 4 μL of DIPEA (final pH 8). The resulting mixture was let stand at room temperature for 48 hours. After completion of the coupling (HPLC, MS), 8 μL of Et₂NH was added to the reaction mixture, and the reaction was stirred for another 2 hours. To the reaction mixture was added 270 μL of 1M TBAF in THF and 2.4 μL of HOAc, and the resulting solution was let stand at room temperature for 1 hour. The reaction was further acidified to a pH of about 3 with 1 M aq. HCl, and the solvent was removed in vacuo. The crude octapeptide was resuspended in 0.1% formic acid in H₂O/MeCN (1:4) and purified using C₁₈ Sep Pak (isolated yield: 5.1 mg, 50% over 3 steps). HPLC (gradient B): t_{R}=18.4 min; λₘₐₓ 290 nm LRMS-ESI (m/z): [M−H]⁻ calcd. for C₄₂H₆₀N₁₁O₁₃S₂, 990.38; found 990.5.

### (t) Synthesis of (N₃-Pro)-bicyclic octapeptide (3)

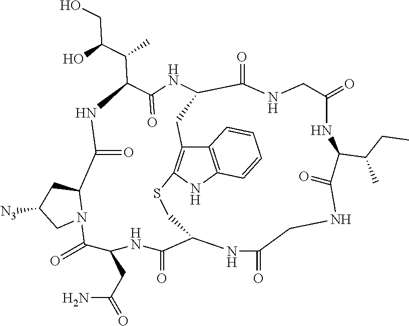 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](N=[N+]=[N-])C[C@H]1C(=O)N[C@@H]([C@@H](C)[C@@H](O)CO)C(=O)N2]

Monocyclic octapeptide 27d (3.81 mg, 4.1 μmol), 1-[bis(dimethylamino)methylene]-1H-1,2,3-triazolo[4,5-b]pyridinium 3-oxide hexafluorophosphate (HATU); 13.4 mg, 35.2 μmol) and DIPEA (6.6 μL) were dissolved in (dimethylacetamide DMA; 0.88 mL). The reaction mixture was let stand at room temperature for 2 hours, at which point 0.9 mL of 0.1% formic acid in H₂O/ACN (80:20) was added to it. This mixture was directly purified by HPLC to afford 1.58 mg of the bicyclic octapeptide 3 (42.2% isolated yield). The HPLC yield for this reaction was higher than the isolated yield, while not wishing to be limited by theory, possibly due to the unwanted retention of the product on the HPLC column. HPLC (gradient C): t_{R}=21.2 min; λₘₐₓ 290 nm HRMS-ESI (m/z): [M+H]⁺ calcd. for C₃₉H₅₄N₁₃O₁₁S, 912.3781; found 912.3787. FIG. 1 shows the ¹H NMR (upper spectrum) and COSY45 (lower spectrum) of 3 (400 MHz, CD₃OD).

### (u) Synthesis of (CN-Pro)-bicyclic octapeptide (4)

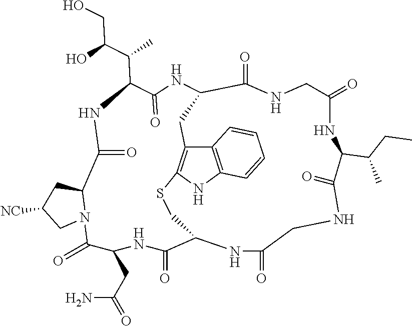 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](C#N)C[C@H]1C(=O)N[C@@H]([C@@H](C)[C@@H](O)CO)C(=O)N2]

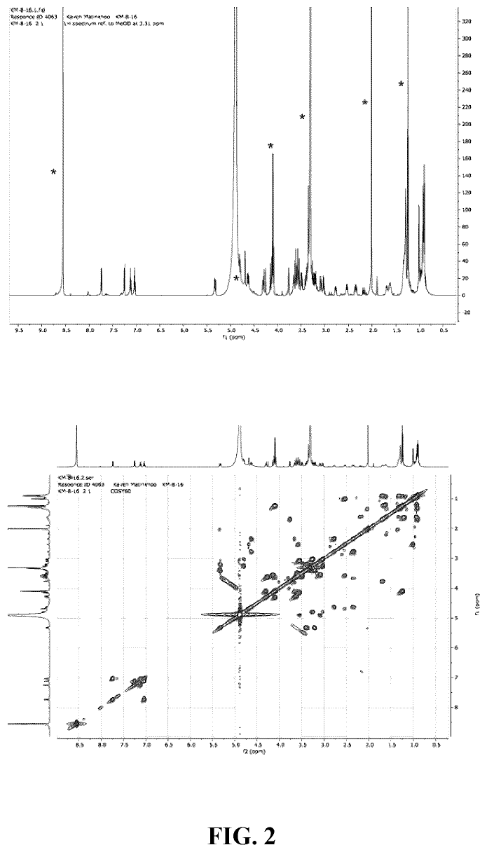

Monocyclic octapeptide 28d (7.5 mg, 8.2 μmol), HATU (25.5 mg, 67.1 μmol) and DIPEA (12.6 μL) were dissolved in DMA (1.5 mL). The reaction mixture was let stand at room temperature for 2 hours, at which point 1.5 mL of 0.1% formic acid in H₂O/ACN (80:20) was added to it. This mixture was directly purified by HPLC to afford 1.08 mg of the bicyclic octapeptide 4 (14.7% isolated yield). The HPLC yield for this reaction was higher than the isolated yield, while not wishing to be limited by theory, possibly due to the unwanted retention of the product on the HPLC column. HPLC (gradient C): t_{R}=19.9 min; λₘₐₓ 290 nm HRMS-ESI (m/z): [M+H]⁺ calcd. for C₄₀H₅₄N₁₁O₁₁S, 896.3719; found 896.3727. FIG. 2 shows the ¹H NMR (upper spectrum) and COSY45 (lower spectrum) of 4 (400 MHz, CD₃OD).

### (v) Synthesis of (SAcm-Pro)-bicyclic octapeptide (8)

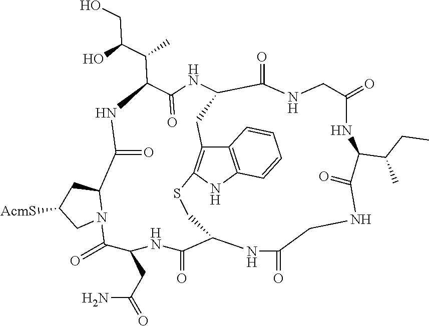

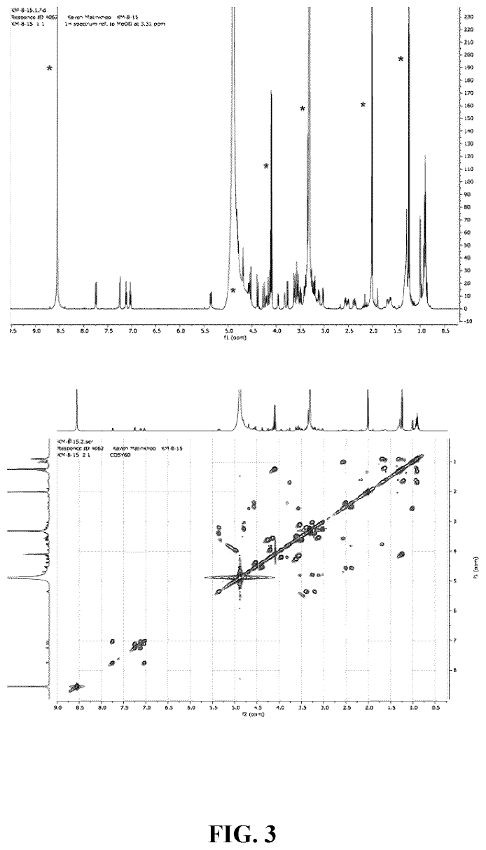

Monocyclic octapeptide 29d (5.1 mg, 5.1 μmol), HATU (16.7 mg, 43.9 μmol) and DIPEA (8.2 μL) were dissolved in DMA (1.1 mL). The reaction mixture was let stand at room temperature for 2 hours, at which point 1.1 mL of 0.1% formic acid in H₂O/ACN (80:20) was added to it. This mixture was directly purified by HPLC to afford 0.82 mg of the bicyclic octapeptide 8 (16.4% isolated yield). The HPLC yield for this reaction was higher than the isolated yield, while not wishing to be limited by theory, possibly due to the unwanted retention of the product on the HPLC column. HPLC (gradient B): t_{R}=19.4 min; λₘₐₓ 290 nm HRMS-ESI (m/z): [M+H]⁺ calcd. for C₄₂H₆₀N₁₁O₁₂S₂, 974.3859; found 974.3840. FIG. 3 shows the ¹H NMR (upper spectrum) and COSY45 (lower spectrum) of 8 (400 MHz, CD₃OD).

### (w) Synthesis of (SH-Pro)-bicyclic octapeptide (5)

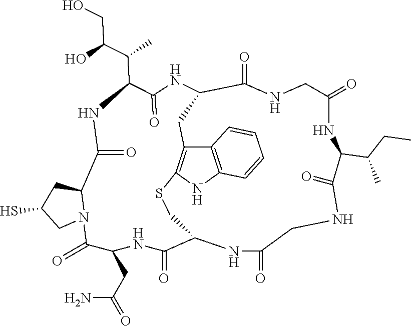 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](S)C[C@H]1C(=O)N[C@@H]([C@@H](C)[C@@H](O)CO)C(=O)N2]

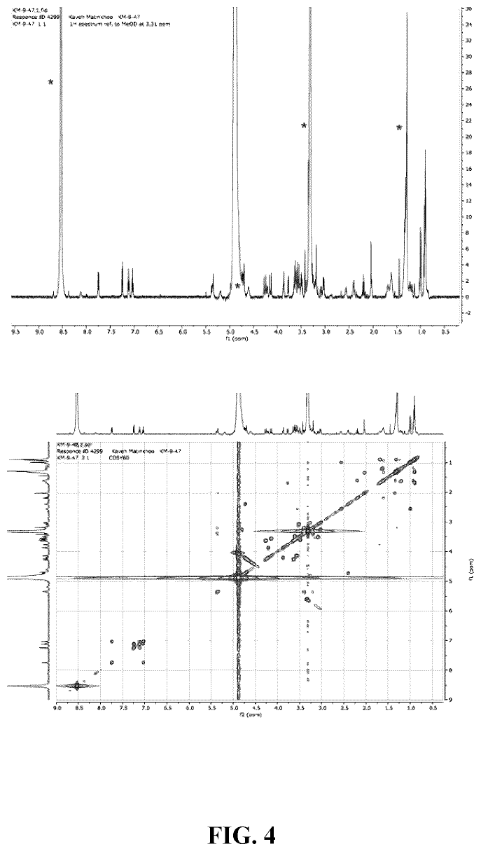

(SAcm-Pro)-bicyclic octapeptide (8, 200 μg, 206 nmol) was added to a solution of PdCl₂ (0.66 mg, 3.7 μmol) in a 6 M aqueous solution of guanidine hydrochloride (Gdn HCl, 200 μL)) at 37° C. (PdCl₂ was used as a stock solution: 9.9 mg of PdCl₂ was dissolved in 3 mL of 6 M aq. Gdn HCl, and 200 μL of this solution was added to the starting material). After 30 minutes, dithiothreitol (DTT; 100 μmol) was added to the reaction for quenching, which formed an orange precipitate in the reaction mixture. The mixture was centrifuged, and the supernatant was diluted with 200 μL of 0.10% formic acid in H₂O/ACN (80:20). The crude solution was directly purified using HPLC to afford (SH-Pro)-bicyclic octapeptide 5. The isolated yield for this reaction varied between 10-40%. While not wishing to be limited by theory, it is believed that the Acm-deprotected product can become trapped in the formed orange precipitate upon addition of DTT. Several rounds of dissolution/sonication were required to extract the product from this precipitate. HPLC (gradient B): t_{R}=16.5 min; λₘₐₓ 290 nm HRMS-ESI (m/z): [M+Na]⁺ calcd. for C₃₉H₅₄N₁₀NaO₁₁S₂, 925.3313; found 925.3293. FIG. 4 shows the ¹H NMR (upper spectrum) and COSY45 (lower spectrum) of 5 (600 MHz, CD₃OD).

### (x) Synthesis of (MeOCHNH-Pro)-bicyclic octapeptide (32)

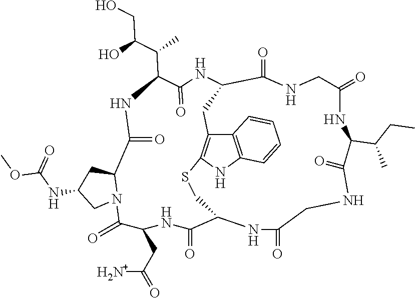 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC([NH3+])=O)C(=O)N1C[C@H](NC(=O)OC)C[C@H]1C(=O)N[C@@H]([C@@H](C)[C@@H](O)CO)C(=O)N2]

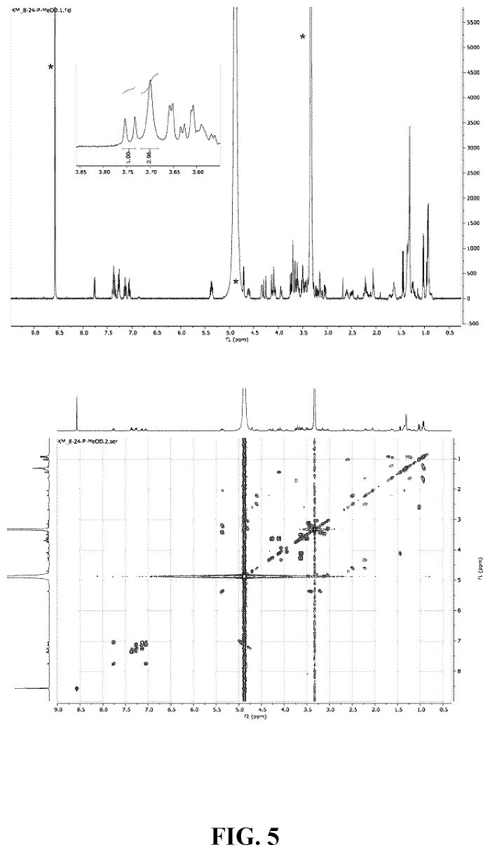

To a solution of N₃-Pro-amanitin (AMA) (3) (500 μg, 550 nmol) in dry DMSO (200 μL) was added PPh₃ (1.5 mg, 6.8 μmol). The reaction mixture was let stand at room temperature for 16 hours, at which point 700 μL of 0.1% formic acid in H₂O was added to the reaction mixture. This mixture was directly purified using HPLC to afford 223 nmol (40% isolated yield) of the speculated methyl carbamate of amino-proline amanitin (32). The HPLC yield for this reaction was higher than the isolated yield, while not wishing to be limited by theory, possibly due to the unwanted retention of the product on the HPLC column. HPLC (gradient C): t_{R}=19.8 min; λₘₐₓ 290 nm HRMS-ESI (m/z): [M+H]⁺ calcd. for C₃₉H₅₈N₁₁O₁₃S, 944.3931; found 944.3939. FIG. 5 shows the ¹H NMR (upper spectrum) and COSY45 (lower spectrum) of 32 (400 MHz, CD₃OD).

### (y) Synthesis of (NH₂-Pro)-bicyclic octapeptide (6)

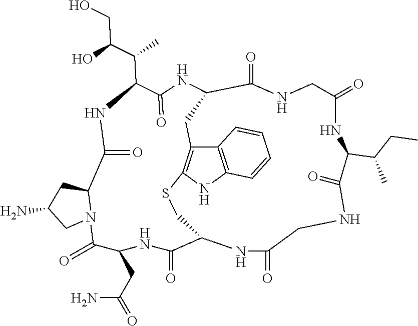 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](N)C[C@H]1C(=O)N[C@@H]([C@@H](C)[C@@H](O)CO)C(=O)N2]

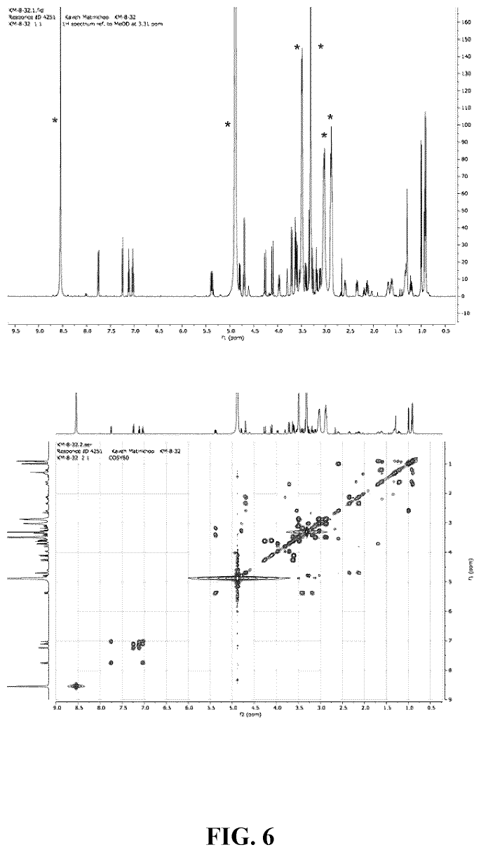

(N₃-Pro)-bicyclic octapeptide 3 (1.58 mg, 1.73 μmol) and DTT (1.34 mg, 8.65 μmol) were dissolved in dry DMSO (1.5 mL). The reaction mixture was let stand at room temperature for 4 hours, at which point 1 mL of 0.1% formic acid in H₂O was added to it. This solution was directly purified using HPLC to afford the product (851 μg, 55.7%). The HPLC yield for this reaction was higher than the isolated yield, while not wishing to be limited by theory, possibly due to the unwanted retention of the product on the HPLC column. HPLC (gradient C): t_{R}=15.2 min; λₘₐₓ 290 nm HRMS-ESI (m/z): [M+H]⁺ calcd. for C₃₉H₅₆N₁₁O₁₁S, 886.3876; found 886.3884. FIG. 6 shows the ¹H NMR (upper spectrum) and COSY45 (lower spectrum) of 6 (600 MHz, CD₃OD).

### (z) Synthesis of (Boc-Gdn-Pro)-bicyclic octapeptide (9)

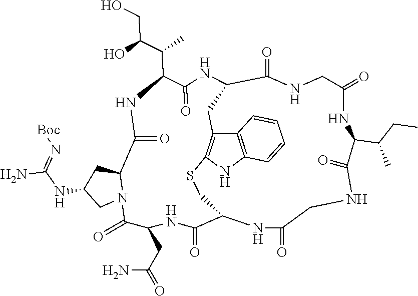 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](N/C(N)=N/C(=O)OC(C)(C)C)C[C@H]1C(=O)N[C@@H]([C@@H](C)[C@@H](O)CO)C(=O)N2]

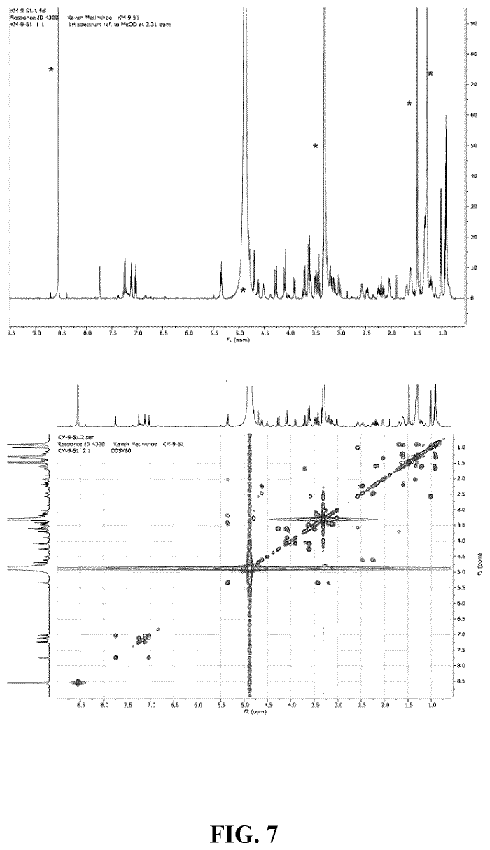

(NH₂-Pro)-Bicyclic octapeptide 6 (708 μg, 800 nmol), N-Boc-N′-TFA-pyrazole-1-carboxamidine (1.47 mg, 4.81 μmol) and DIPEA (DIPEA was added as a stock solution: 1 μL of DIPEA was dissolved in 1 mL DMSO, and 275 μL of this solution was added to the reaction; 283 nL) were dissolved in THF/DMSO (550 μL/275 μL). The resulting solution was let stand at room temperature for 24 hours, at which point MeOH (177 μL), H₂O (71 μL) and K₂CO₃ (1.42 mg, 10.3 μL) were added. After 3 hours, the pH of the reaction mixture was adjusted to 3 by adding 1 M aq. HCl, and the volatiles were evaporated in vacuo. The resulting mixture was re-suspended in 400 μL of 0.1% formic acid in H₂O/ACN (80:20) and was directly purified using HPLC to afford 308 μg (37.5%) of the product. The HPLC yield for this reaction was higher than the isolated yield, while not wishing to be limited by theory, possibly due to the unwanted retention of the product on the HPLC column. HPLC (gradient C): t_{R}=19.3 min; λₘₐₓ 290 nm LRMS-ESI (m/z): [M+H]⁺ calcd. for C₄₅H₆₆N₁₃O₁₃S, 1028.5; found 1028.8. FIG. 7 shows the ¹H NMR (upper spectrum) and COSY45 (lower spectrum) of 9 (600 MHz, CD₃OD).

### (aa) Synthesis of (Gdn-Pro)-bicyclic octapeptide (7)

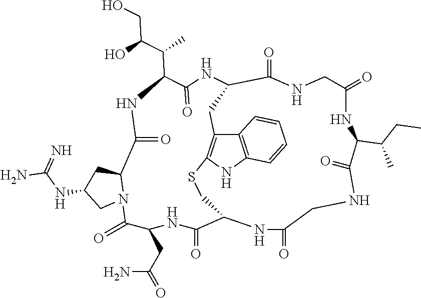 [SMILES: CC[C@H](C)[C@@H]1NC(=O)CNC(=O)[C@@H]2Cc3c([nH]c4ccccc34)SC[C@H](NC(=O)CNC1=O)C(=O)N[C@@H](CC(N)=O)C(=O)N1C[C@H](NC(=N)N)C[C@H]1C(=O)N[C@@H]([C@@H](C)[C@@H](O)CO)C(=O)N2]

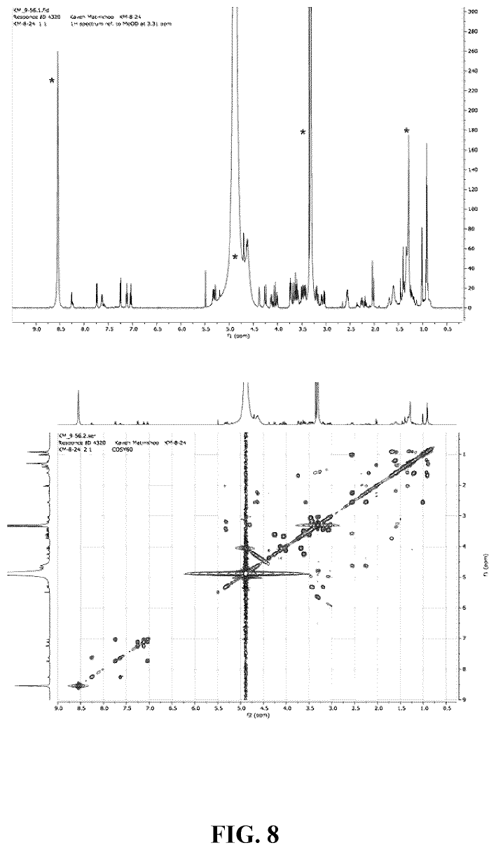

A mixture of TFA/DCM (120 μL/240 μL) was added to (Boc-Gdn-Pro)-bicyclic octapeptide 9 (300 nmol), and the resulting solution was let stand at room temperature for 1 hour. At this point, the volatiles were removed under reduced pressure and the resulting residue was re-suspended in 400 μL of 0.1% formic acid in H₂O/ACN (80:20) and directly purified using HPLC to afford the product (180 nmol, 60%). The HPLC yield for this reaction was higher than the isolated yield, while not wishing to be limited by theory, possibly due to the unwanted retention of the product on the HPLC column. HPLC (gradient C): t_{R}=15.3 min; λₘₐₓ 289 nm HRMS-ESI (m/z): [M+H]⁺ calcd. for C₄₀H₅₈N₁₃O₁₁S, 928.4094; found 928.4101. FIG. 8 shows the ¹H NMR (upper spectrum) and COSY45 (lower spectrum) of 7 (600 MHz, CD₃OD).

### III. Results

Owing to greater synthetic ease, “dideoxy-amanitin” analogs with a tryptathionine staple lacking the (R)-sulfoxide were prepared herein. This rationale was predicated on confirming through total synthesis and previously published results obtained on the natural product and synthetic analogs that the thioether is equipotent with respect to the (R)-sulfoxide.^{17,28} In terms of the 6-hydroxy group, the naturally occurring amaninamide, which retains the (R)-sulfoxide but lacks the 6-hydroxytryptathionine, is equipotent,²⁹ corroborating a well-established notion that the 6-hydroxytryptathionine can be replaced with tryptathionine. Furthermore, synthetic derivatives that have been based on the more synthetically accessible tryptathionine staple also show near-native potencies^{4,19,20} and an N-propargyl(Asn) analog lacking the sulfoxide and hydroxytryptathionine showed only 3-4 fold lower potency.^{12(d)} Hence, it was in the context of a “dideoxy” amanitin scaffold that new (4R)-hydroxyproline (Hyp) analogs were evaluated.

The conformational bias of trans-(4R)-hydroxyproline that has been studied in terms of its conformational bias favoring the more sterically occluded Cγ-exo conformer was considered in the design. Extensive studies on collagen have also evaluated conformationally biased (4R)-substituted prolines and these works have led to questioning the contribution of H-bonding by the Hyp to embedded water molecules or to carbonyls on opposing collagen strands.³⁰ Indeed, (4R)-substituted prolines that are incapable of H-bonding, e.g. 4-fluoro-, 4-chloro-, 4-azido-proline form highly stable collagen helices^{31,32} as reviewed,³³ suggesting that ring-pucker contributions are more important to determining the structural integrity of collagen. Yet in devising new Hyp analogs for use in amanitin, this conformational bias contributes to the challenge of analog development because of the need to address clearly defined H-bonds to Glu822 and His1085 with a suitable functional group, while also ensuring a Cγ-exo conformation that is seen in the crystal structures of α- and β-amanitin.^{19,28,34} To probe these H-bonds while generally seeking to maintain the conformational bias of Hyp, five trans-4-substituted Hyp analogs into amanitin were prepared, each substituted with thiol, azide, amine (ammonium), cyano, and guanidine (guanidinium). Additionally, two intermediates, Acm-protected trans-4-mercapto-proline and tert-butoxycarbonyl (Boc)-protected trans-4-guanidino-proline were investigated along with a trans-4-methylcarbamoyl-proline, that adventitiously formed upon azide reduction, all of which provide further H-bonding functionalities (Table 1).

TABLE 1
Analogs of Hyp for incorporation into amanitin.

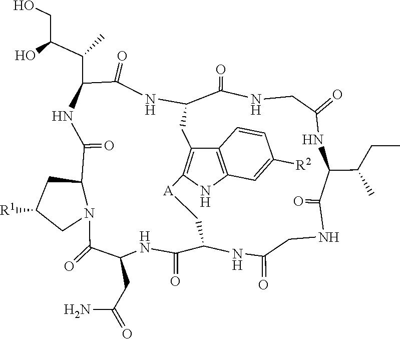

| Compound* | R¹ | A | R² |
|---|---|---|---|
| 1 | OH | (R)—SO | OH |
| 2 | OH | S | H |
| 3 | N₃ | S | H |
| 4 | CN | S | H |
| 5 | SH | S | H |
| 6 | NH₂ | S | H |
| 7 | Gdn | S | H |
| 8 | SAcm | S | H |
| 9 | Boc-Gdn | S | H |
| 32 | MeOCONH | S | H |

*Compound 1 represents the natural product, α-amanitin, while 2 shows dideoxy-α-amanitin, which is the basis for the synthetic analogs. Compound 32 is an adventitious by-product.

Two factors were considered in selecting these analogs: (i) potential for H-bond donation or acceptance and (ii) the ring pucker induced by the presence of a trans-4-substitutent on the proline residue. Given limitations in throughput, readily available prolines that would provide key information as to the role of Hyp in toxicity were sought. Of these, the cyano-, amino-, mercapto-, and guanidino-analogs appeared compelling due to their potential to form at least one H-bond with the neighboring Glu822 or His1085. It was further anticipated that the guanidine analog would form bifurcated H-bonds or alternatively a salt-link with Glu822.³⁵ The use of the amino-proline was rationalized similarly in terms of favorable electrostatic interactions. Exploration of the mercapto-proline was also justified based on the potential for creating disulfide-conjugated or other immolating linkers that would readily undergo bioreduction upon cytosolic internalization.

In terms of ring-pucker, whereas the mercapto-proline prefers a Cγ-endo conformation of approximately 5:1 ratio,³⁶ both the amino- and guanidino-prolines (extant as ammonium and guanidinium cations) have been reported to strongly favor a Cγ-exo conformation.³⁷ Trans-4-amido prolines as well as carbamoylated ones (N-Boc-modified) have also been reported to adopt a Cγ-exo conformation.³⁸,3⁹ The cyano-proline, which is strongly electron withdrawing and, while not wishing to be limited by theory, expected to favor the Cγ-exo conformation, has the understated potential to accept an H-bond, albeit weakly.⁴⁰ Finally, the azido-proline, which has been reported to favor the Cγ-exo conformation,⁴¹ provides a control for conformation in the absence of H-bonding. The 4-fluoroproline derivative was prepared but not carried forward in this analysis as it was not found to be cytotoxic.

The synthesis of the Hyp analogs began with preparation of cis-N^{α}-Boc-4-hydroxyproline methyl ester (cis-Boc-Hyp-OMe, 12) from the commercially available trans-N^{α}-Boc-4-hydroxyproline (Boc-Hyp, 10): Boc-Hyp was subjected to an intramolecular Mitsunobu reaction using PPh₃ and DIAD to afford the lactone of cis-Hyp 11. An azide-assisted saponification of this lactone with methanol afforded the methyl ester of cis-Boc-Hyp 12.⁴² Subsequently, the hydroxyl group was mesylated and the resulting mesylated compound was subjected to S_{N}2 conditions to yield the desired trans-isomer of various analogs (Scheme 2).

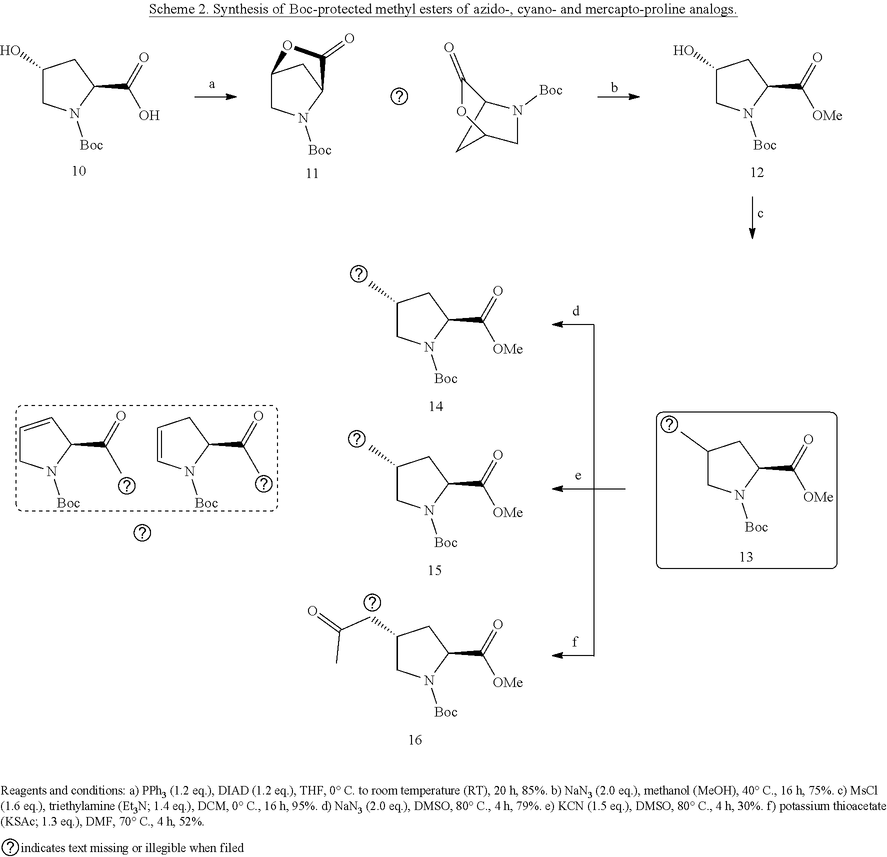

For the synthesis of azido- and cyano-proline analog, their corresponding methyl esters were saponified to the free acid, and the Boc protecting group was swapped with a fluorenylmethoxycarbonyl (Fmoc) protecting group to yield solid phase peptide synthesis (SPPS) compatible monomers 21 and 22 (Scheme 3, A). In the case of mercapto-proline, the thioacetate group and the methyl ester of 16 were concomitantly saponified, the Boc protecting group was removed, then an Acm (acetamidomethyl) protecting group was introduced on the free thiol. Finally, the free amine was protected with Fmoc to yield the fully protected monomer 26 (Scheme 3, B).

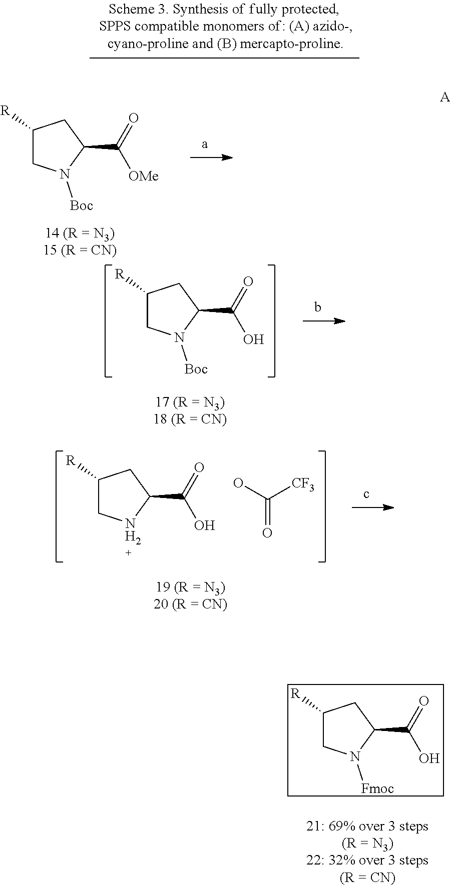

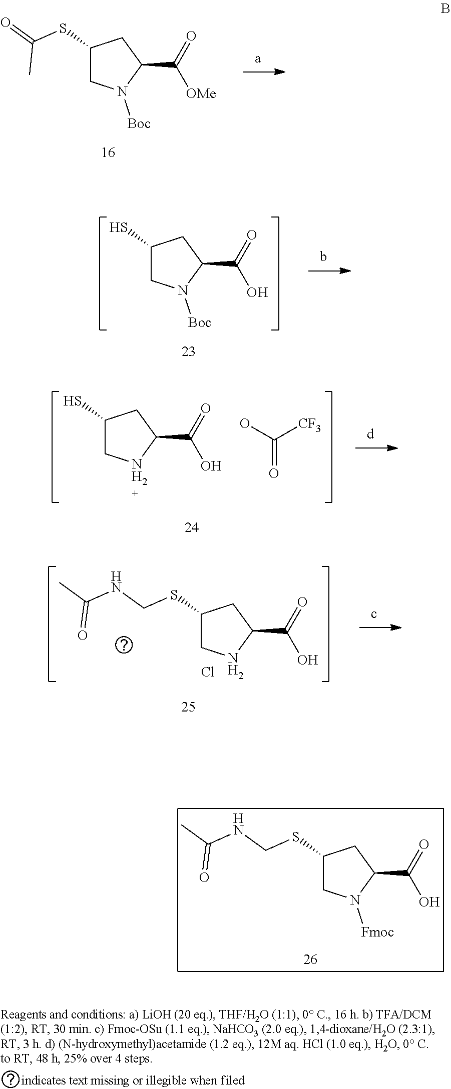

Following a solid phase strategy similar to the previously reported total synthesis of α-amanitin, and reproducing the synthesis of the (2S,3R,4R)-dihydroxyisoleucine residue 31, the three aforementioned monomers were incorporated into the corresponding dideoxy-amanitins (Scheme 4). To summarize, the synthetic proline analogs were separately loaded on 2-chlorotrityl chloride (CTC) resin, followed by the coupling of Fmoc-Asn(Trt)—OH, Fmoc-Cys(Trt)-OH, Fmoc-Gly-OH, Fmoc-Ile-OH, Fmoc-Gly-OH and Boc-Fpi-OH (30), where Fpi=3a-fluoro-hexahydropyrrolo-[12,3-b]indoline,⁴³ to afford the linear heptapeptide. Treating the resin with TFA/DCM (1:1) resulted in the concomitant, global deprotection of the acid-labile protecting groups and tryptathionine formation via the Savige-Fontana reaction⁴⁴ to yield monocyclic heptapeptides of various amanitin analogs (27c, 28c, 29c). Next, the activated, fully protected, SPPS compatible DHIle 31 was grafted onto the N-terminus, followed by in situ deprotection of Fmoc and TBS protecting groups. The resulting monocyclic octapeptides (27d, 28d, 29d) were macrolactamized to yield the final bicyclic octapeptides containing different Hyp analogs (3, 4 and 8).

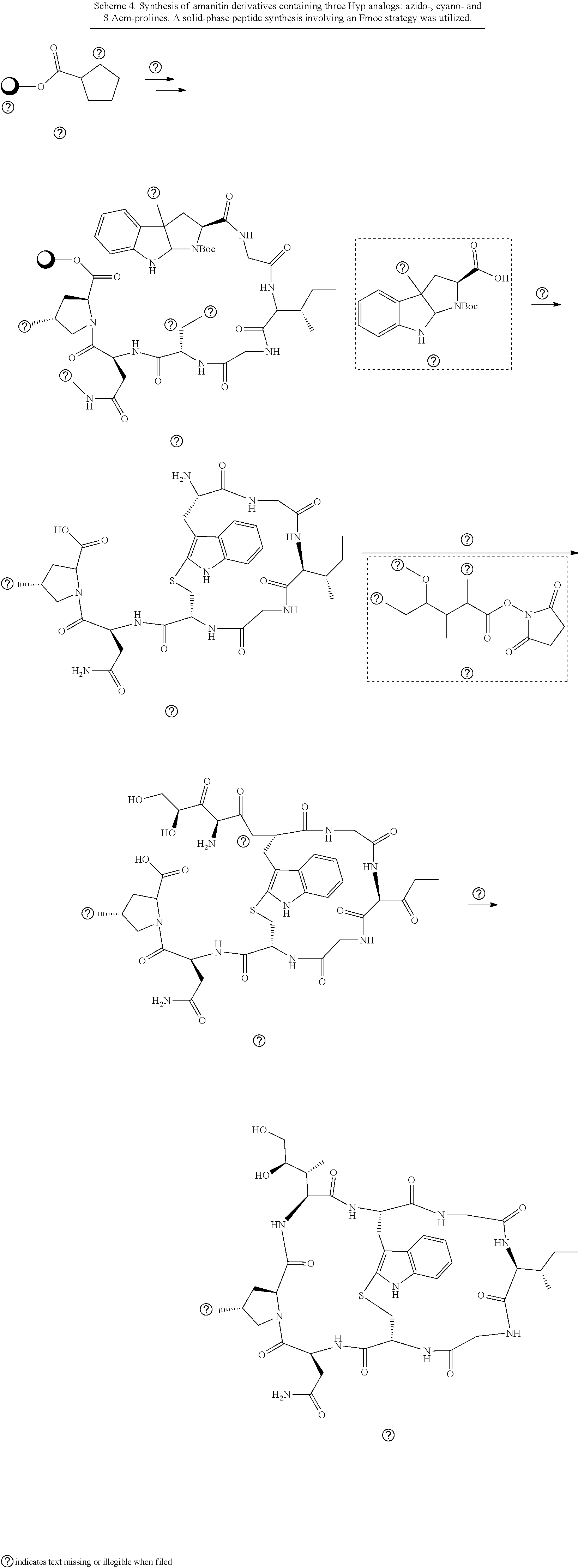

To synthesize an amanitin analog containing the unprotected mercapto-proline residue 5, the Acm-protected intermediate 8 was treated with a large excess of PdCl₂ in 6M aq. guanidinium hydrochloride (Gdn-HCl) as the solvent, for 30 minutes at 37° C., followed by quenching with dithiothreitol, DTT (Scheme 5, A).⁴⁵

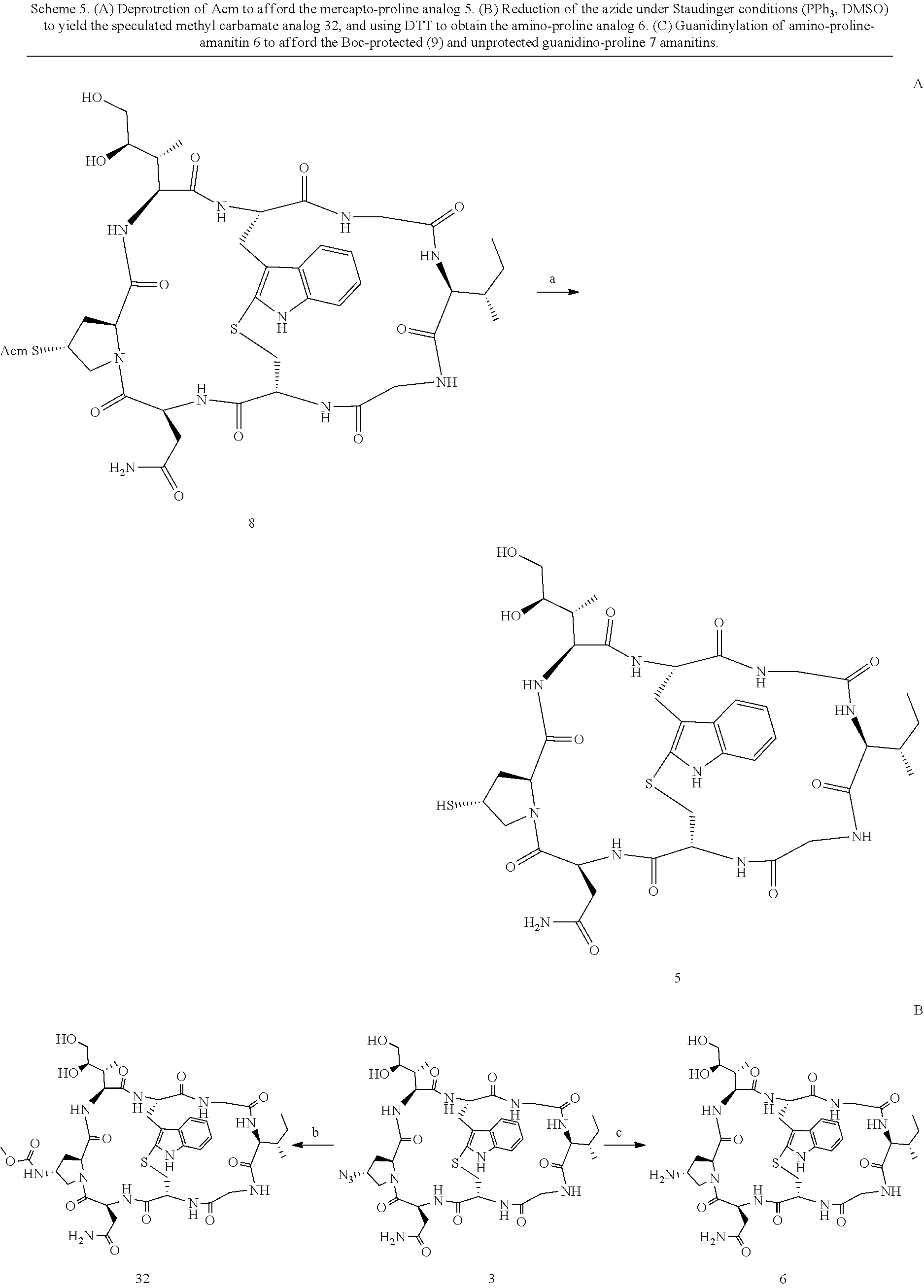

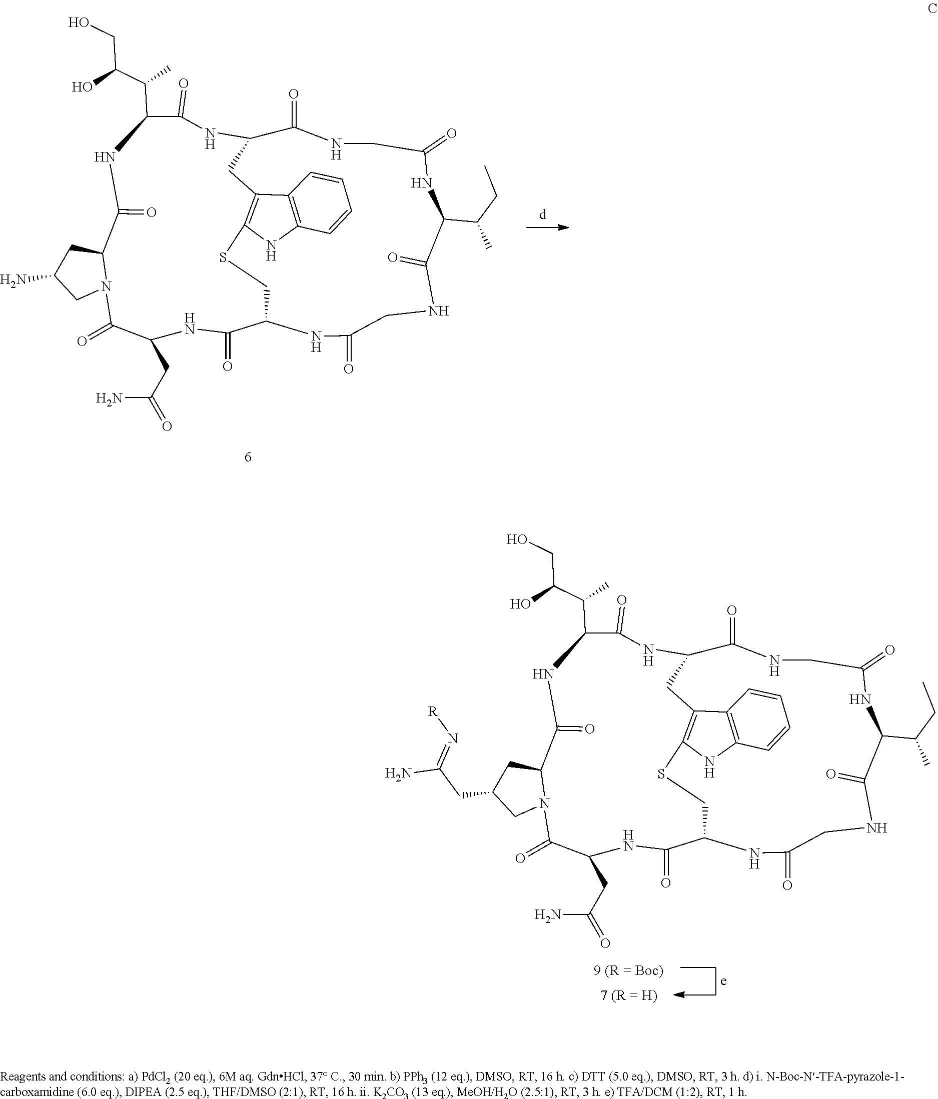

To prepare the amino-proline analog from the azido-prolyl amanitin, Staudinger reduction conditions were initially employed using PPh₃ in aqueous DMSO.⁴⁶ Surprisingly, instead of the desired amino analog, a by-product was obtained, that based on the mass spectrometry and preliminary ¹H-NMR studies, was characterized as the methyl carbamate of the amino-proline residue (32). There is literature precedent for the reaction of organic azides with triphenyl phosphine and C₀₂ to give isocyanates that may in turn react with solvent nucleophiles to yield an array of new functional groups.^{47,48} While not wishing to be limited by theory, Scheme 6 shows a proposed mechanism for the formation of the by-product resulting from the reaction of N₃-Pro-AMA with PPh₃ in wet DMSO. While not wishing to be limited by theory, residual methanol in the reaction might be responsible for the nucleophilic attach on the isocyanate intermediate to yield the methylcarbamate product (in Scheme 6, R^{P}=the proline residue on dideoxy-amanitin). This derivative was also incorporated into the panel of analogs for biochemical characterization.

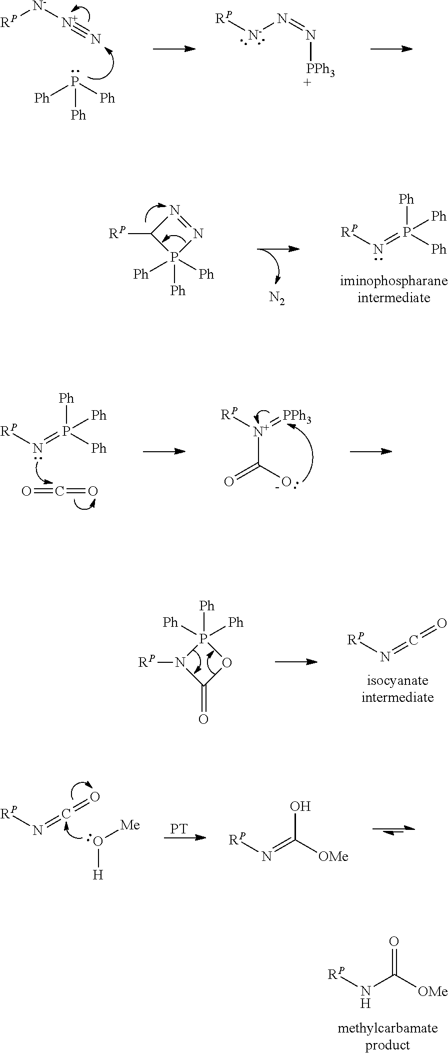

Scheme 6. Proposed mechanism for the formation of the methylcarbamate by-product (32).

Attempting alternative conditions for the reduction of the azido-proline residue to the corresponding amine, the azide group on 3 was successfully reduced to an amine 6 using DTT in DMSO (Scheme 5, B).⁴⁹ Lastly, to prepare the guanidino analog 7, the amino-proline-containing amanitin 6 was reacted with N-Boc-N′-TFA-pyrazole-1-carboxamidine in THF/DMSO in the presence of DIPEA.⁵⁰ The resulting doubly protected guanidino-moiety was treated with K₂CO₃ in MeOH and H₂O to remove the TFA protecting group, affording intermediate 9. Finally, the Boc protecting group was removed using TFA in DCM (1:1) to yield an amanitin analog containing a guanidino-proline residue (7) (Scheme 5, C).

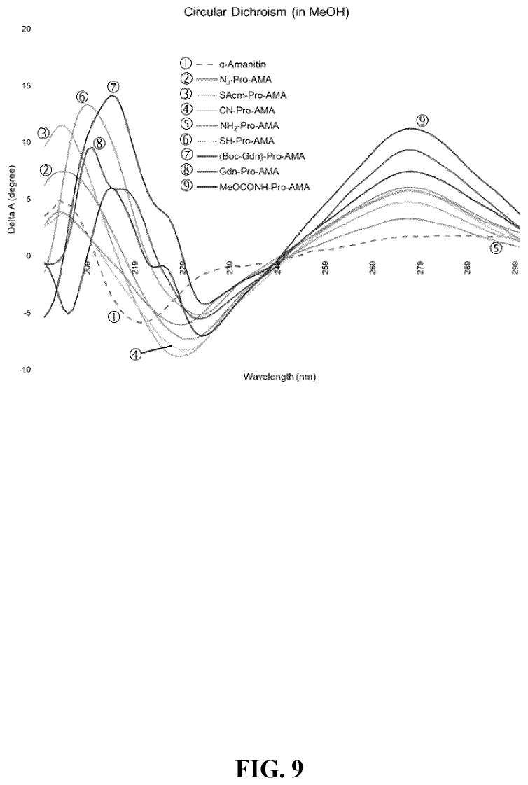

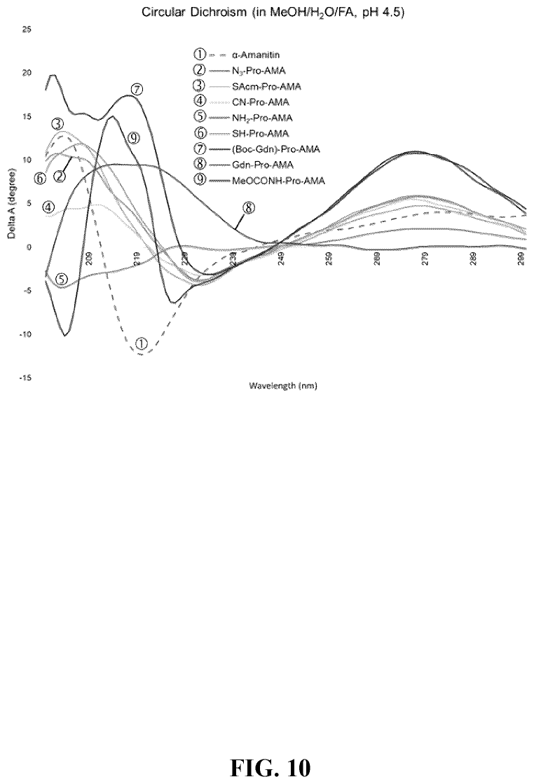

At this stage, eight amanitin analogs that contained various analogs of the hydroxyproline residue were assembled and purified. As a general measure to compare the overall conformation of these amanitins, circular dichroism (CD) spectra of all the analogs were obtained in two solvents: 1) MeOH, and 2) MeOH/(0.1% formic acid in H₂O) (10:1) at pH 4.5. Most analogs exhibited similar CD spectra in methanol (FIG. 9), with a negative cotton effect generally observed between 215 and 250 nm, and a positive cotton effect above 250 nm. However, curiously, in the acidic environment of the second solvent system (MeOH/H₂O at pH 4.5), only four analogs (N₃-, CN-, Acm-S- and mercapto-proline amanitins) demonstrated similar CD spectra, while the other proline-modified amanitins showed unpredictable spectral behavior (FIG. 10).

CHO (Chinese hamster ovary) cells can provide a convenient means of studying cytotoxicity as, for example, they are readily killed by α-amanitin (Kᵢ about 0.5 μM) even though they do not overexpress the organic anion-transporting protein (OATP) that has been implicated in active toxin transport.⁵¹ To assess the cytotoxicity of these synthetic analogs, CHO cells were treated with various concentrations of said analogs (generally ranging from 0.078 to 20 μM), and the percentages of viable cells were measured using an MTT colorimetric assay (Tables 2-10).

TABLE 2
Viability assay against CHO cells (MTT colorimetric assay) for
α-amanitin (1).

| | % viable cells | | |
|---|---|---|---|
| Concentration (M) | n = 1 | n = 2 | n = 3 |
| 0 (DMSO) | 86 | 99 | 114 |
| 9e-009 | 89 | 91 | 117 |
| 2.7e-008 | 84 | 90 | 109 |
| 8.2e-008 | 76 | 73 | 97 |
| 2.5e-007 | 76 | 82 | 81 |
| 7.4e-007 | 59 | 65 | 81 |
| 2.22e-006 | 16 | 13 | 14 |
| 6.67e-006 | 2 | 2 | 1 |
| 2e-005 | 1 | 1 | 1 |

TABLE 3
Viability assay against CHO cells (MTT colorimetric assay) for
N₃-Pro-amanitin (3).

| | % viable cells | | |
|---|---|---|---|
| Concentration (M) | n = 1 | n = 2 | n = 3 |
| 0 (DMSO) | 107 | 92 | 101 |
| 1.56e-007 | 101 | 89 | 88 |
| 3.125e-007 | 99 | 89 | 91 |
| 6.25e-007 | 101 | 93 | 87 |
| 1.25e-006 | 105 | 88 | 89 |
| 2.5e-006 | 104 | 95 | 88 |
| 5e-006 | 104 | 88 | 89 |
| 1e-005 | 107 | 100 | 94 |
| 2e-005 | 112 | 112 | 109 |

TABLE 4
Viability assay against CHO cells (MTT colorimetric assay) for
CN-Pro-amanitin (4).

| | % viable cells | | |
|---|---|---|---|
| Concentration (M) | n = 1 | n = 2 | n = 3 |
| 0 (DMSO) | 109 | 97 | 95 |
| 1.56e-007 | 102 | 93 | 87 |
| 3.125e-007 | 99 | 92 | 90 |
| 6.25e-007 | 102 | 89 | 92 |
| 1.25e-006 | 102 | 91 | 92 |
| 2.5e-006 | 101 | 91 | 87 |
| 5e-006 | 103 | 92 | 90 |
| 1e-005 | 110 | 101 | 90 |
| 2e-005 | 121 | 118 | 108 |

TABLE 5
Viability assay against CHO cells (MTT colorimetric assay) for
SAcm-Pro-amanitin (8).

| | % viable cells | | |
|---|---|---|---|
| Concentration (M) | n = 1 | n = 2 | n = 3 |
| 0 (DMSO) | 95 | 97 | 109 |
| 1.56e-007 | 94 | 93 | 113 |
| 3.125e-007 | 88 | 93 | 110 |
| 6.25e-007 | 93 | 90 | 108 |
| 1.25e-006 | 85 | 92 | 106 |
| 2.5e-006 | 87 | 92 | 105 |
| 5e-006 | 86 | 89 | 109 |
| 1e-005 | 90 | 99 | 109 |
| 2e-005 | 110 | 105 | 99 |

TABLE 6
Viability assay against CHO cells (MTT colorimetric assay) for
SH-Pro-amanitin (5).

| | % viable cells | | |
|---|---|---|---|
| Concentration (M) | n = 1 | n= 2 | n =3 |
| 0 (medium) | 105 | 97 | 98 |
| 7.8e-008 | 94 | 91 | 89 |
| 1.56e-007 | 93 | 94 | 93 |
| 3.125e-007 | 94 | 91 | 90 |
| 6.25e-007 | 98 | 92 | 95 |
| 1.25e-006 | 93 | 92 | 94 |
| 2.5e-006 | 91 | 94 | 99 |
| 5e-006 | 100 | 90 | 96 |
| 1e-005 | 94 | 100 | 95 |
| 2e-005 | 106 | 98 | 97 |

TABLE 7
Viability assay against CHO cells (MTT colorimetric assay) for
MeOCOHN-Pro-amanitin (32).

| | % viable cells | | |
|---|---|---|---|
| Concentration (M) | n = 1 | n = 2 | n = 3 |
| 0 (medium) | 93 | 97 | 109 |
| 7.8e-008 | 88 | 89 | 97 |
| 1.56e-007 | 88 | 87 | 96 |
| 3.125e-007 | 89 | 87 | 94 |
| 6.25e-007 | 91 | 91 | 98 |
| 1.25e-006 | 89 | 92 | 93 |
| 2.5e-006 | 88 | 89 | 89 |
| 5e-006 | 92 | 88 | 87 |
| 1e-005 | 86 | 88 | 96 |
| 2e-005 | 99 | 103 | 102 |

TABLE 8
Viability assay against CHO cells (MTT colorimetric assay) for
NH₂-Pro-amanitin (6).

| | % viable cells | | |
|---|---|---|---|
| Concentration (M) | n = 1 | n = 2 | n =3 |
| 0 (medium) | 106 | 97 | 97 |
| 7.8e-008 | 96 | 89 | 91 |
| 1.56e-007 | 95 | 86 | 97 |
| 3.125e-007 | 86 | 92 | 91 |
| 6.25e-007 | 92 | 85 | 87 |
| 1.25e-006 | 95 | 87 | 88 |
| 2.5e-006 | 86 | 85 | 82 |
| 5e-006 | 87 | 79 | 84 |
| 1e-005 | 91 | 81 | 83 |
| 2e-005 | 92 | 89 | 85 |

TABLE 9
Viability assay against CHO cells (MTT colorimetric assay) for
Boc-Gdn-Pro-amanitin (9).

| | % viable cells | | |
|---|---|---|---|
| Concentration (M) | n = 1 | n = 2 | n = 3 |
| 0 (medium) | 92 | 96 | 113 |
| 3.9e-008 | 88 | 88 | 102 |
| 7.8e-008 | 85 | 88 | 101 |
| 1.56e-007 | 87 | 84 | 97 |
| 3.125e-007 | 82 | 90 | 103 |
| 6.25e-007 | 84 | 87 | 102 |
| 1.25e-006 | 84 | 89 | 100 |
| 2.5e-006 | 86 | 84 | 100 |
| 5e-006 | 86 | 87 | 104 |
| 1e-005 | 103 | 98 | 119 |

TABLE 10
Viability assay against CHO cells (MTT colorimetric assay) for
Gdn-Pro-amanitin (7).

| | % viable cells | | |
|---|---|---|---|
| Concentration (M) | n = 1 | n = 2 | n = 3 |
| 0 (medium) | 99 | 101 | 100 |
| 7.8e-008 | 96 | 92 | 92 |
| 1.56e-007 | 88 | 91 | 80 |
| 3.125e-007 | 85 | 86 | 88 |
| 6.25e-007 | 88 | 83 | 84 |
| 1.25e-006 | 89 | 86 | 86 |
| 2.5e-006 | 90 | 82 | 90 |
| 5e-006 | 93 | 85 | 85 |
| 1e-005 | 100 | 102 | 102 |
| 2e-005 | 99 | 101 | 100 |

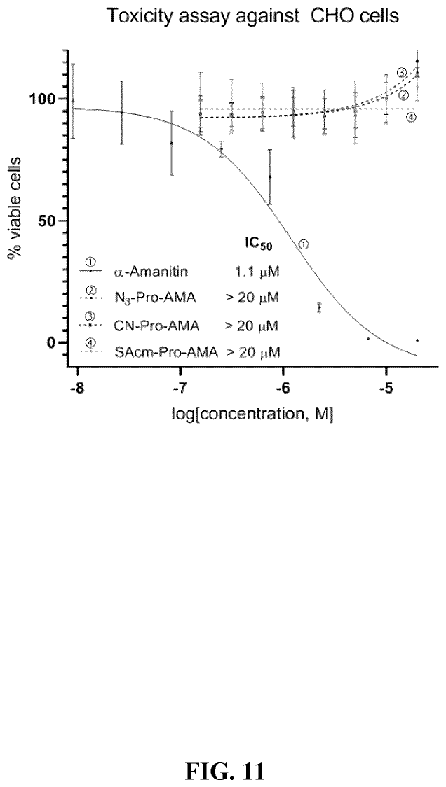

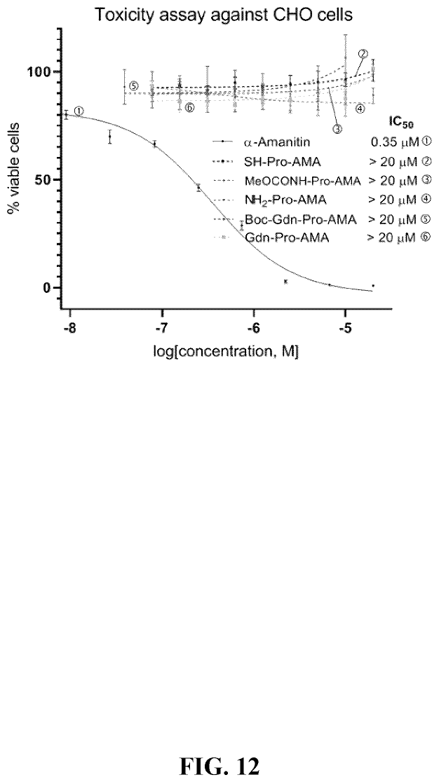

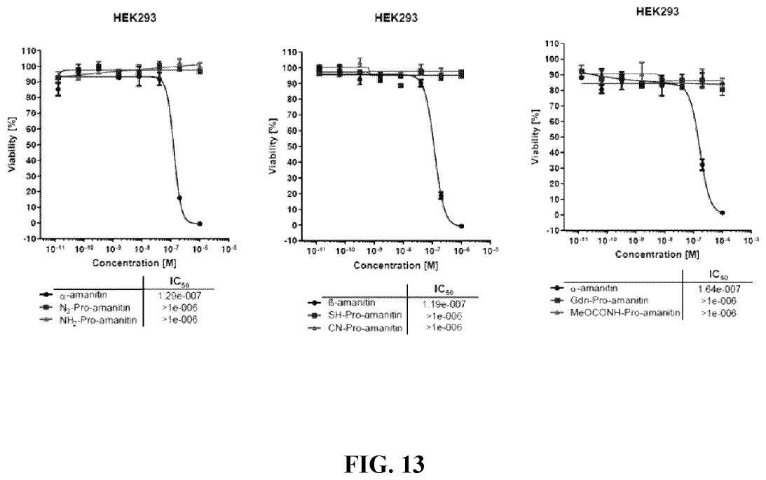

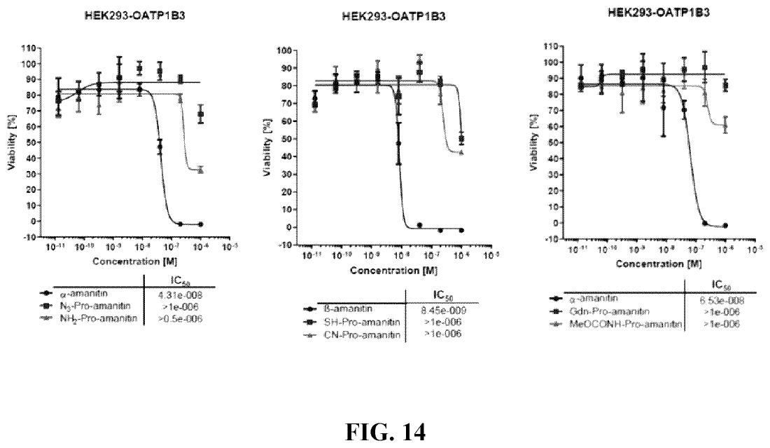

Surprisingly, none of the synthetic analogs exhibited any appreciable level of toxicity towards CHO cells, even at concentrations as high as 20 μM (FIG. 11 and FIG. 12; Tables 2-10). As a control, the IC₅₀ values of 0.35-1.1 μM obtained for α-amanitin in this assay were in line with the values previously reported.^{4,12(d),18,19,24} In a separate experiment, the unprotected analogs (3, 4, 5, 6, 7 and 32) were tested against human embryonic kidney 293 (HEK293) cells, with and without overexpression of the OATP1B3 protein, to investigate the effect of this anion-transporting protein on the cytotoxicity of the synthetic analogs. While some analogs showed slightly elevated toxicity against the HEK293 cells that overexpressed OATP1B3, this increase was marginal, and no conclusive IC₅₀ values could be obtained for these analogs (FIG. 13 and FIG. 14).

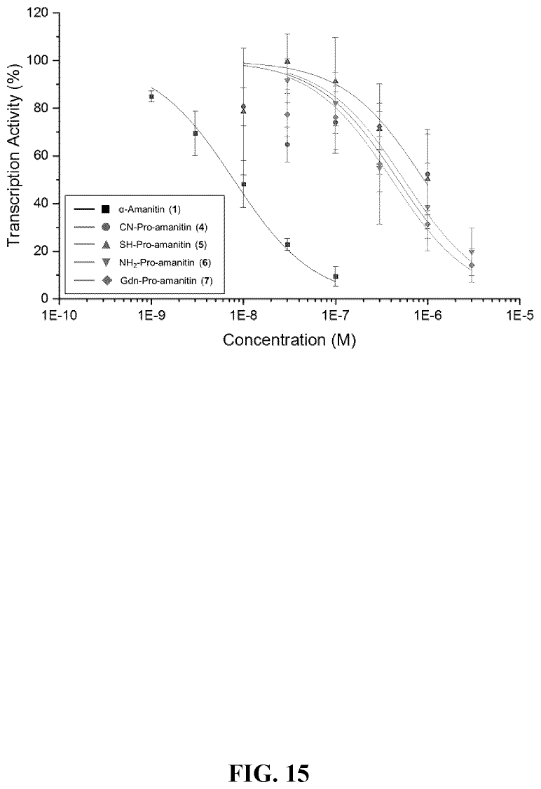

Surprised by these findings, it was questioned whether poor internalization of certain derivatives, in particular with respect to 6 (amino-proline) and 7 (guanidino-proline), resulted in apparent lack of toxicity. To address this hypothesis, all synthetic analogs (with the exception of the presumed MeOCONH-proline analog 32) were subjected to an in vitro transcription assay using HeLaScribe® nuclear extract containing Pol II and all other transcription factors using a DNA template containing a strong cytomegalovirus (CMV) promoter.⁵² To test the inhibition of Pol II by α-amanitin, dose-escalating concentrations of toxin (1 to 100 nM) were added to the transcription reaction and an inhibition curve for α-amanitin was obtained, with an IC₅₀ value of 7.9±0.9 nM that matched the reported values in the literature.^{53,54} The in vitro activity of all synthetic analogs was measured at various concentrations (generally ranging from 10 nM to 3 μM) and inhibition curves for most of the tested analogs were obtained (FIG. 15). A full IC₅₀ curve was not acquired for the analogs that did not exhibit any appreciable inhibitory effects up to 1 μM. The results for the transcription assay of amanitin analogs containing various proline residues are summarized in Tables 11 and 12.

TABLE 11
Transcription activities of HeLa nuclear extract in varying amanitin
analog concentrations.

| Con- | | | | | |
|---|---|---|---|---|---|
| cen- | | | | | |
| tra- | Transcription activity E (%) | | | | |
| tion | α-amanitin | CN-Pro- | SH-Pro- | NH₂-Pro- | Gdn-Pro- |
| (nM) | (1) | AMA (4) | AMA (5) | AMA (6) | AMA (7) |
| 1 | 85.0 ± 2.4 | — | — | — | — |
| 3 | 69.5 ± 9.4 | — | — | — | — |
| 10 | 48.1 ± 9.8 | 80.7 ± 7.9 | 78.5 ± 26.6 | — | — |
| 30 | 22.9 ± 2.6 | 64.8 ± 7.4 | 99.6 ± 11.6 | 91.7 ± 9.3 | 77.3 ± 9.0 |
| 100 | 9.5 ± 4.1 | 74.0 ± 13.0 | 91.2 ± 18.5 | 82.3 ± 12.8 | 76.1 ± 13.2 |
| 300 | — | 72.4 ± 9.7 | 71.2 ±19.0 | 55.0 ± 23.6 | 56.6 ± 11.6 |
| 1000 | — | 52.3 ± 17.0 | 50.3 ± 20.8 | 38.6 ± 18.3 | 31.3 ± 5.8 |
| 3000 | — | — | — | 19.8 ± 9.9 | 14.1 ± 7.1 |
| IC₅₀ | 7.9 ± 0.9 | 480 ± 380 | 910 ± 330 | 550 ± 90 | 410 ± 80 |
| (nM) | | | | | |
| R² | 0.991 | −5.762 | 0.860 | 0.983 | 0.918 |

TABLE 12
Results of the in vitro transcription assay for α-amanitin and
synthetic analogs using the HeLa nuclear extract (n = 3).

| Compound* | R¹ | A | R² | IC₅₀ (nM) | R² |
|---|---|---|---|---|---|
| 1 | OH | (R)-SO | OH | 7.9 ± 0.90 | 0.991 |
| 3 | N₃ | S | H | >1000 | — |
| 4 | CN | S | H | 480 ± 380 | −5.760 |
| 5 | SH | S | H | 910 ± 330 | 0.860 |
| 6 | NH₂ | S | H | 550 ± 90 | 0.983 |
| 7 | Gdn | S | H | 410 ± 80 | 0.918 |
| 8 | SAcm | S | H | >1000 | — |
| 9 | Boc-Gdn | S | H | >600 | — |
| 32 | MeOCONH | S | H | N/A | N/A |

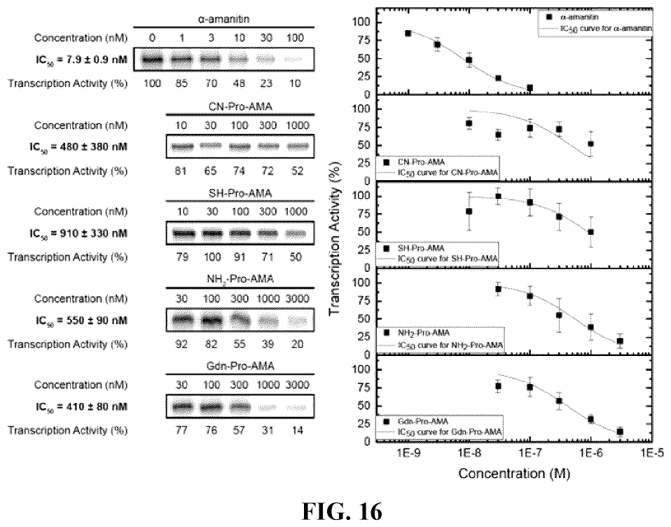

Of all synthetic analogs, 7 (guanidine) was the most active inhibitor with an IC₅₀ value of about 400 nM, which is approximately 50-fold higher than that of the natural product. Other potentially active analogs include amino-proline 6 (IC₅₀=550±90 nM) and mercapto-proline 5 (IC₅₀=910±230 nM). Analogs that did not exhibit any noticeable inhibitory effects up to 1 μM concentrations include N₃-, SAcm- and Boc-guanidino-pro amanitins. All compounds were investigated three times (n=3) except for 4 (n=4), which proved exceptional in terms of its inhibitory effects on Pol II leading to an unexplained but reproducible inability to fit an IC₅₀ curve to the data points making curve-fitting especially difficult (FIG. 15 and FIG. 16). While not wishing to be limited by theory, based on the transcription results for the mercapto-proline analog, with an IC₅₀ value of 910±230 nM (R²=0.860), the possibility of disulfide formation to dimers of 5 or other side products cannot be excluded even though 5 was synthesized fresh and immediately subjected to the transcription assay in buffer that contained DTT.

For amanitin analogs with IC₅₀ of 600 nM or above, upon rearrangement of equation 1, with transcription activity E,

$${IC}_{50} = ( \frac{E}{1 - E} ) [ I ]$$

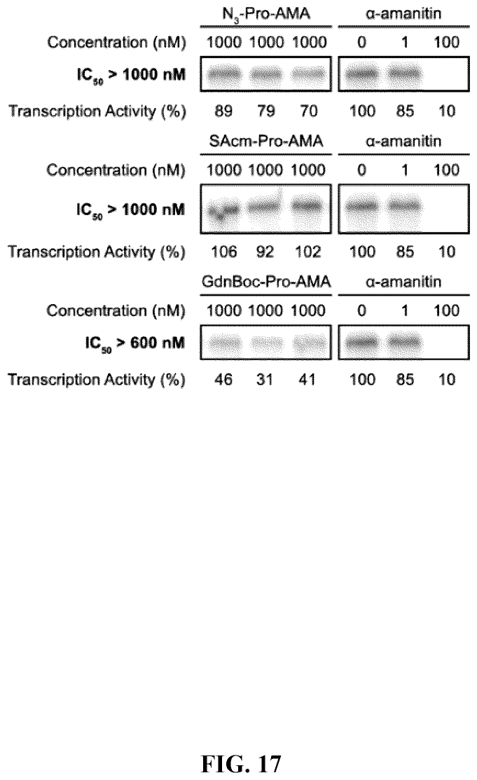

Using [I]=1.0 μM, the averaged transcription activities Ē was used to estimate high values of IC₅₀. FIG. 17 shows a [³²P]-autoradiogram of transcription runoff assay resolved by 8% denaturing PAGE.

### IV. Discussion and Conclusions

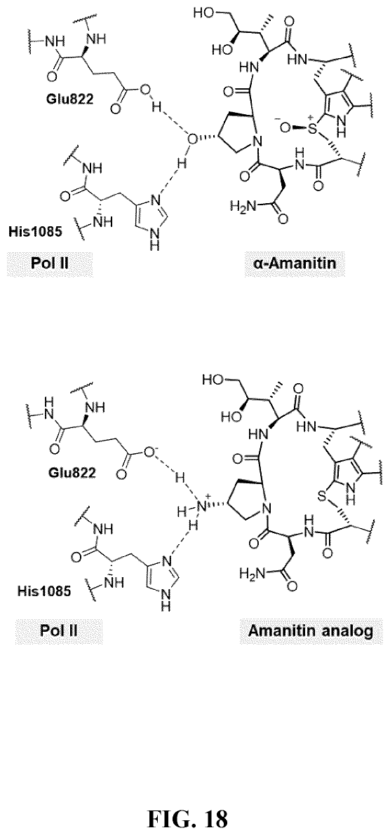

Among the Hyp analogs that were synthesized and incorporated into the structure of dideoxy-amanitin, surprisingly none was as active as the natural product, α-amanitin, as assayed in CHO-based cytotoxicity assays and by run-off in vitro transcription assays that remove the variable of poor internalization. The installation of various substituents at the C-4 position of proline in a trans orientation can impose changes in the ring pucker, which in turn may globally affect the 3D structure of the corresponding amanitin by analogy to other model peptides.^{55,56} In support of the notion that these Hyp analogs may impart long-range yet subtle conformational effects, the obtained CD spectra suggest that the overall 3D structures of the synthetic analogs differ from that of the natural product, but generally resemble each other, at least in methanol (FIG. 9). Nevertheless, at pH 7.4, α-amanitin and all the synthetic analogs generally show a positive cotton effect past 250 nm, yet behave differently at wavelengths below 250 nm with the synthetic analogs exhibiting a local minimum at 230-234 nm, while α-amanitin shows a local minimum at 220 nm. Greater differences were observed when the spectra were acquired at pH 4.5 (FIG. 10). Regarding the question of H-bonding at the binding site within Pol II, to date, cryo-EM and XRD structures have not directly observed the toxin to sufficient resolution and have been refined in accord with the ground-state structure determined by Lipscomb and coworkers for β-amanitin.^{57,58} Yet hydrogen bonding is possibly the most evident motif that can impact the activity of the synthesized analogs. As shown by Kornberg^{14,59,60} and Cramer^{15,16,61} in several reports, the most notable interaction of α-amanitin with Pol II is an H-bond between the hydroxyl group of Hyp and the glutamic acid residue (A822 Glu) of Pol II (FIG. 18).

Nevertheless, the thermodynamic gain afforded by these H-bonding interactions must be questioned given that the toxin must be desolvated from bulk solvent wherein the same H-bonding interactions occur. Notwithstanding the overall thermoneutrality of H-bonding in this case, we reasoned that the (4R)-amino-, (4R)-guanidino-, (4R)-methylcarbamoyl-, and even (4R)-cyano-proline could all be recognized by Glu822 and/or His1085, either through H-bonding, charge-charge complementarity, or both. As a control, the (4R)-azido-proline was used that is incapable of H-bonding yet should afford the Cγ-exo conformation that proline cannot. With respect to the mercapto-prolyl-amanitin, we recognized that the thiol may serve as a possible analog of a hydroxyl group yet is a poor H-bond donor and incapable of accepting an H-bond.^{23,62} While not wishing to be limited by theory, with a preference for Cγ-endo ring puckering in this case, such may explain the generally poor inhibitory activity of this analog. Protected versions including the Boc-Gdn-proline and Acm-mercapto-proline were also evaluated as these present additional functionalities capable of further H-bonding interactions that may found supplementary interactions within the binding site. The methylcarbamoyl-proline proved inactive despite the strong potential for H-bonding and an expected Cγ-exo conformation. We expected lower IC₅₀ values for NH₂- and Gdn-Pro amanitins due to their suspected ability to form H-bonds or salt bridges with A822 Glu residue of Pol II at the physiological pH. As these functional groups most certainly exist in their cationic form (NH₃⁺ and GdnH⁺, respectively), which would prevent them from acting as H-bond acceptors, they could still form salt bridges with the Glu822 while donating an H-bond to His1085.

In considering the Cγ-exo conformation of these two cationic derivatives, along with the azide- and cyano-prolines favor, the energetic difference between the exo and endo forms for (4R)-fluoroproline is about −1 kcal/mol at 25° C., which translates to an exo endo ratio of about 6,⁶³ a ratio that extends to all derivatives except the mercapto-proline. While the Cγ-exo conformation favors a trans-amide bond, the trans-geometry is already found within the strained macrolactam backbone. Hence, all toxin analogs except that bearing the mercapto-proline should have mimicked the conformation of the natural product. The fact that 6 (amino) and 7 (guanidino) were the strongest inhibitors of Pol II in this series may indeed point to the potential for accessing the interactions seen in the crystal and cryo-EM structures while assuming the correct Cγ-exo conformation.

The in vitro toxicity of α-amanitin and the synthetic analogs was determined in HEK293 (FIG. 13) and in HEK293-OATP1B3 cells that overexpress the organic anion-transporting polypeptide 1B3 (OATP1B3) (FIG. 14) to assess whether OATP1B3 mediates active transport of α-amanitin and the Hyp analogues of amanitin into the cell.

Human embryonic kidney HEK293 cells lack the OATP1B3 transporter (wt-HEK293) and hence showed only micromolar susceptibility to α-amanitin (IC₅₀=0.13±0.08 μM) and β-amanitin (IC₅₀=0.12±0.09 μM), while the amanitin derivatives showed no cytotoxic effect. On the contrary, transfected HEK293 cells constitutively expressing OATP1B3 (HEK293-OATP1B3) showed clearly enhanced sensitivity to α-amanitin, with an IC₅₀ value of 43.1±7.4 nM. The 3-fold increase in toxicity of α-amanitin on HEK293-OATP1B3 cells relative to HEK293 cells indicates that this toxin is subject to OATP1B3-mediated transport. β-amanitin is an even better substrate for the OATP1B3 transporter as indicated by a 15-fold reduction of the IC₅₀ value to 8.5±1.1 nM.

Hyp-substituted amanitin analogs mediated no cytotoxic effect on wt-HEK293 cells and a very mild cytotoxic effect only at high concentrations in the μM range on HEK293-OATP1B3 cells. For example, at the highest used concentration of 1 μM, cell viability of NH₂-Pro-amanitin 6 showed the lowest value of 33%, in comparison to N₃-Pro-amanitin (68%), SH-Pro-amanitin (50%), CN-Pro-amanitin (43%), Gdn-Pro-amanitin (86%), MeOCONH-Pro-amanitin (610%), α-amanitin (0%) and β-amanitin (0%). While not wishing to be limited by theory, this slightly lower cell viability of OATP1B3-overexpressing cells compared to wt-HEK cells after exposure to Hyp analogs at identical concentrations could be an indication that these variants are only partially transported into the cell via the organic anion-transporting polypeptide 1B3 (OATP1B3). However, in direct comparison to α-amanitin, these variants exhibit substantially lower cytotoxicity on these overexpressing cells compared to the natural compound. While not wishing to be limited by theory, this reduced cytotoxic potential could be attributed to (i) a reduced binding affinity to RNA Pol II and/or (ii) limited uptake into the cell via the OATP1B3 transporter. The correlation between the inhibitory effect on Pol II and the cytotoxic potency on HEK-OATP1B3 cells of the different Hyp analogs supports the assumption that Pol II binding affinity plays a decisive role in mediating cytotoxicity. This finding is in line with the absence of any cytotoxic effect on wt-HEK cells; assuming unspecific and thus comparable uptake of all variants into these cells lacking the OATP1B3 transporter.

On the other hand, the inhibitory activity of Hyp variants on Pol II was more comparable to the effect of α-amanitin in contrast to the cytotoxic potency on HEK-OATP1B3 cells which deviated by more than two orders of magnitude from the potency of α-amanitin. While not wishing to be limited by theory, this observation suggests that the Hyp variants represent poor/unfavorable substrates for the OATP1B3 transporter. While not wishing to be limited by theory, in the case of some variants, this could be explained by the cationic form predominant at physiological pH (e.g. R—NH₃⁺ and R-GdnH⁺).

As can be seen from the results of the present Example, the Hyp residue is critical for the cytotoxicity and inhibitory activity of α-amanitin and is not readily replaceable with analogs known to mimic the conformational bias of Hyp while in certain cases complementing the H-bonding interactions seen amanitin-bound structures of Pol II. These studies addressed the role of the Hyp by testing analogs thereof, four of which were chosen for Cγ-exo conformation seen with Hyp. None of the synthetic toxins replaced Hyp with a synthetic mimic having similar activity. Nevertheless, replacing the hydroxyl group on Hyp with, for example, an amine, guanidine, cyanide or thiol may provide useful chemical handles for the design of bioreducible and self-immolating handles. If potency is effectively augmented by the use of a targeting agent such as an antibody, lower cytotoxicity may, for example, prove advantageous for medicinal applications in cancer patients, for example, if the less-potent composition provided a chemical handle for further mediating biological activation. Hence the amine, guanidine, cyanide and thiol may, for example, be useful in the design of new amanitin-bioconjugates. ADCs incorporating the NH₂-Pro and CN-Pro were synthesized and tested on cells (Example 2).

### Example 2: Synthesis and Biological Evaluation of Bioconjugates of Select Amanitin Analogs

### I. Materials and Methods

Analytical HPLC chromatograms were generated on a VWR-Hitachi Chromaster system equipped with an auto injector and a diode array detector. Analytical injections were performed on a Phenomenex Luna 10 μm C18(2) column (4.6×250 mm) fitted with a column guard at a flow rate of 1.4 mL/min. Chromatograms were recorded at 210 and 290 nm and evaluated after blank correction. Preparative HPLC purification was performed on a VWR LaPrepΣ-System equipped with an auto injector, variable wavelength detector and fraction collector on a Phenomenex Axia-packed Luna 10 μm C18(2) column (21.2×250 mm) fitted with a column guard at a flow rate of 30 mL/min. Chromatograms were recorded at 290 nm and peaks were collected by automated slope dependent peak recognition with EZ Chrome software. Analytical and preparative methods use the same gradient, with a linear adjustment of the flow. Gradient J: Solvent A=0.05% trifluoroacetic acid in water, solvent B=acetonitrile. 0-3 min 5%-30% B; 3-26 min 30%-44% B; 26-27 min 44%-100% B; 27-29 min 100% B; 29-30 min 100%-5% B; 30-32 min 5% B. Gradient K: Solvent A=pure water, solvent B=acetonitrile. 0-15 min 5%-100% B; 15-18 min 100% B; 18-18.5 min 100%-5% B; 18.5-22 min 5% B.

Antibody design and expression: The anti-HER2antibody (T-D265C) is based on the humanized antibody Trastuzumab (Herceptin® Roche) developed by Genentech. T-D265C, the THIOMAB derivative of Trastuzumab, was generated by introducing a cysteine residue into the heavy chain (D265C) of trastuzumab, thereby providing T-D265C antibody with two defined, favorable positions for payload attachment. The antibody was expressed in Expi293 cells (Life Technologies, Carlsbad, CA, USA) using transient transfection methods.

Tumor cell lines/cell culture: Human cancer cell lines SK-BR-3, SK-OV-3, and JIMT-1 (HER2⁺) and MDA-MB-231 (HER2⁻) were obtained from ATCC (Manassas, VA, USA), or from DSMZ (Braunschweig, Germany). All cell lines were cultured as recommended and periodically tested for contamination with mycoplasma by PCR. Cell lines were authenticated using Multiplex Cell Authentication by Multiplexion (Heidelberg, Germany). The SNP profiles matched known profiles or were unique.

In vitro cytotoxicity assay of ADCs: The potency of HER2-targeted T-D265C-amatoxin conjugates was evaluated in vitro on a number of HER2⁺ cancer cell lines, including SK-BR-3, SK-OV-3, and JIMT-1 cells. Cells were seeded at a density of 2×10³ cells/well in 96-well plates. After a 24 h growth period, treatment was performed using a concentration series of the T-D265C amanitin conjugates starting at 100 nM (1:5 serial dilution). One row of cells on each plate was kept untreated as a negative control. 96 h post treatment, cell viability was determined using the BrdU incorporation assay (Cell Proliferation ELISA, BrdU (colorimetric) Kit, Roche) according to manufacturer's instructions. Chemiluminescence was measured using a 96-well plate reader (FLUOstar Optima, BMG LABtech, Ortenberg, Germany). All measurements were taken in triplicates and raw counts were normalized as the percentage of signal relative to untreated cells. Sigmoidal dose-response curve fitting for IC₅₀ calculation was performed using GraphPad Prism 8 (GraphPad Software, Inc., La Jolla, CA). Prism 8.4.2 software (GraphPad Software, San Diego, CA, USA) was used to perform statistical tests.

### II. Synthetic Procedures

### (a) Synthesis of (NH₂-Pro)-dideoxy-Ama MP-Val-Ala-PAB-linker (34)

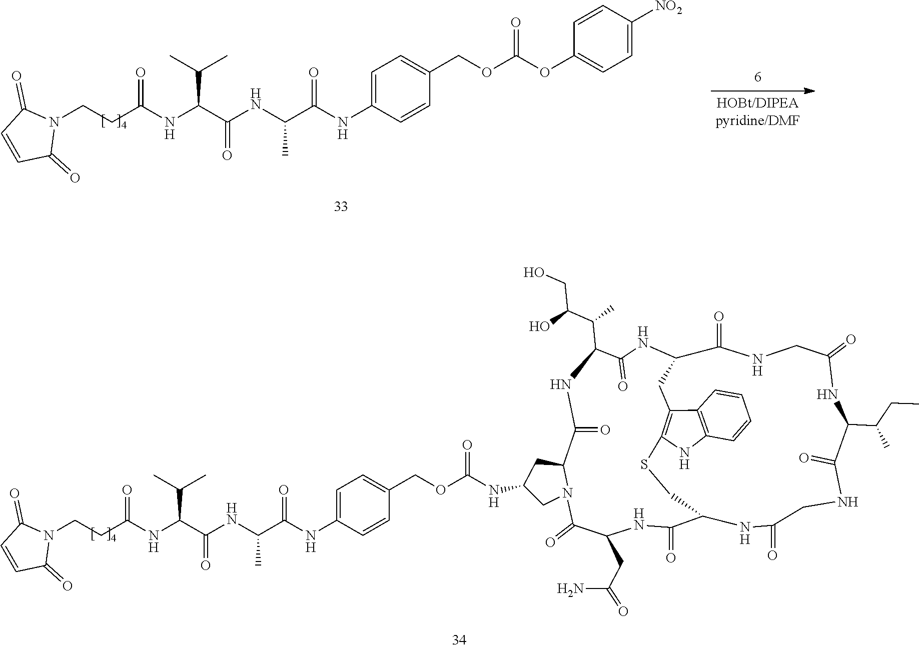

(NH₂-Pro)-bicyclic octapeptide 6 (1.15 mg, 1.3 μmol) was dissolved in abs. pyridine/DMF 4:1 (v/v). MP-Val-Ala-PAB-PNP 33 (1.27 mg, 1.95 μmol) was dissolved in 35 μL of a 0.24 M HOBt solution in DMF and added to the amine, followed by DIPEA (0.443 μL, 1.95 μmol). The reaction mixture was stirred at room temperature for 23 h, then filtrated though a centrifugal filter with 0.2 μm nylon membrane and purified by preparative reversed-phase high-performance liquid chromatography (RP-HPLC). The peak corresponding to the target material was collected and lyophilized to afford the product (1.00 mg, 55%) as a white powder. HPLC (gradient J): t_{R}=13.54 min; λₘₐₓ 286 nm. LRMS-ESI (m/z): [M+H]⁺ calcd. for C₆₅H₈₈N₁₅O₁₈S, 1399.6; found 1399.7.

### (b) Synthesis of (CN-Pro)-bicyclic octapeptide Fmoc-Val-Ala-PAB-linker (36)

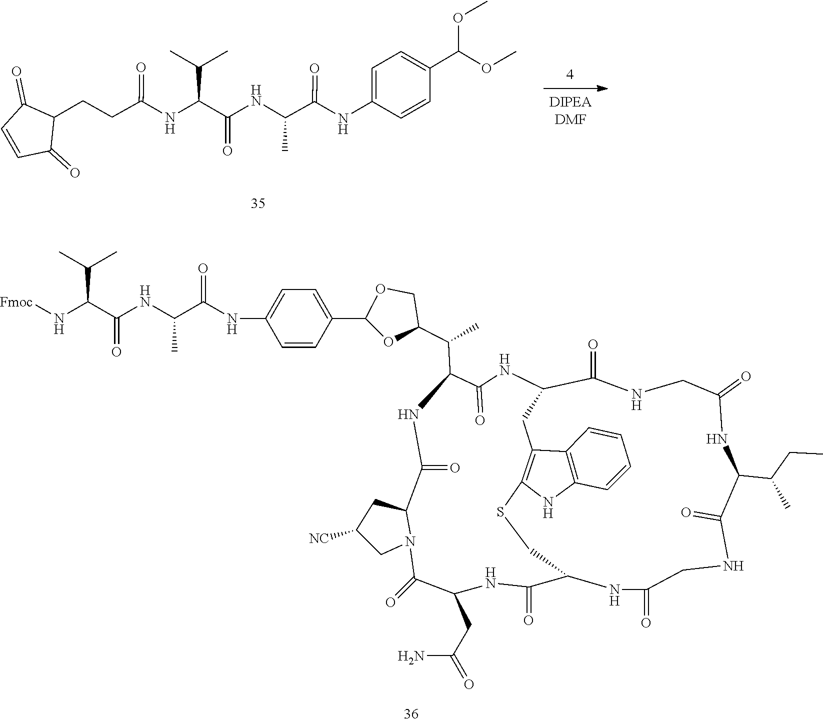

(CN-Pro)-bicyclic octapeptide 4 (3.14 mg, 3.5 μmol) was dissolved in 1 mL dry DMF and evaporated to dryness to remove traces of water. A solution of Fmoc-Val-Ala-PAB-dimethyl acetal 35 (19.6 mg, 10 eq.) in 1 mL dry DMF was added, followed by 10 μL TFA. The reaction mixture was stirred on a rotary evaporator at room temperature and 250 mbar for 30 min followed by evaporation to dryness. The residue was re-dissolved in 1 mL DMF. A sample of 0.25 μL was diluted in 50 μL of 1% triethylamine in methanol and analyzed on HPLC (gradient K) to show 33% conversion to the cyclic acetal. The process of stirring and evaporation was repeated five times with 250 μL (2.5 eq) aliquots of additional 35 solution until a conversion of 95% was reached. The residue was then dissolved in 400 μL DMF and dropped into 10 mL of ice cooled tert-butyl methyl ether (MTBE) containing 0.1% triethylamine. After resting for 10 min on ice, the suspension was centrifuged at 3900×g, the supernatant was discarded, the precipitated solids were resuspended in a second portion of MTBE/Et₃N before being centrifuged again and dried in vacuo. The crude pellet was dissolved in 400 μL DMF and purified on preparative HPLC (gradient K). The peak eluting from 11.58-12.22 min was collected and lyophilized to afford the product (3.05 mg, 72%) as a white solid. HPLC (gradient K): t_{R}=12.29 min; λₘₐₓ 256 nm, 299 nm. LRMS-ESI (m/z): [M+H]⁺ calcd. for C₇₀H₈₃N₁₄O₁₅S, 1391.6; found 1391.6.

### (c) Synthesis of (CN-Pro)-bicyclic octapeptide MP-Val-Ala-PAB-linker (37)

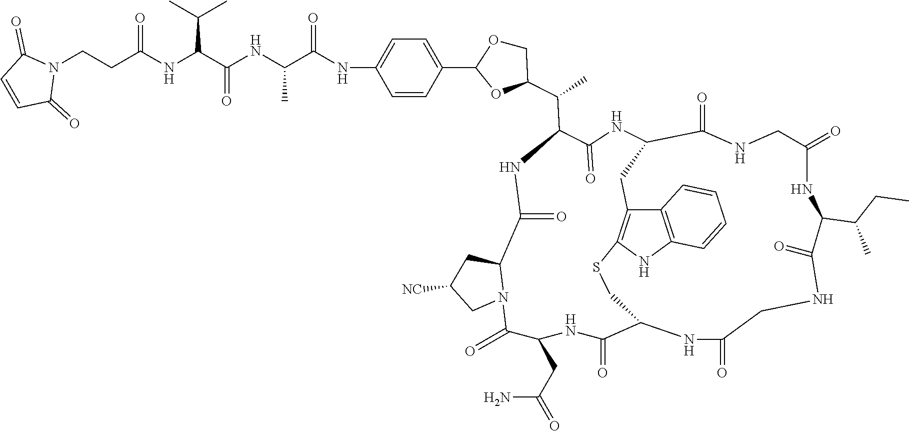

Intermediate 36 (3.05 mg, 2.19 μmol) was dissolved in 500 μL DMF. Diethylamine (15.46 μL, 150 μmol) was added and the resulting mixture was stirred for 15 min at room temperature and evaporated to dryness afterwards. HPLC (gradient K) showed complete Fmoc deprotection and the dibenzofulvene byproduct was removed by dissolving the crude in 400 μL methanol and dropping into 10 mL MTBE. Isolation and washing of the precipitate were performed by centrifugation as described herein above in the previous step II(b). The resulting pellet of amino intermediate was dissolved in a 15 mL centrifugal tube with a solution of 3-(maleimido)propionic acid N-hydroxysuccinimide ester (BMPS, 1.17 mg, 4.38 μmol) in 250 μL DMF. DIPEA (0.75 μL, 4.38 μmol) was added and the tube was shaken overnight. MTBE (10 mL) was added to the tube and the precipitate was washed and isolated as above. The crude product was then dissolved in 200 μL methanol and applied to prep. HPLC (gradient K). The product peak at 8.67-9.17 min was collected and freeze-dried to result in 1.61 mg (56%) maleimide product as a white powder. HPLC (gradient K): t_{R}=9.41 min; λₘₐₓ 289 nm. LRMS-ESI (m/z): [M+H]⁺ calcd. for C₆₂H₇₈N₁₅O₁₆S, 1320.55; found 1321.7.

### (d) Synthesis of T-D265C-Amatoxin Antibody Conjugates

The cysteine reactive linker-amanitin compounds NH₂-Pro-amanitin-VA linker (35) and CN-Pro-amanitin-VA linker (37) were site-specifically conjugated to the engineered cysteine residues (D265C) of anti-HER2 THIOMAB using maleimide chemistry. In brief, T-D265C in PBS pH 7.4 at 5 mg/mL was reduced with tris(2-carboxyethyl)phosphine hydrochloride (TCEP), and following 2 dialysis steps, inter-chain disulfides were re-oxidized by dehydroascorbic acid. Subsequently, the cysteine-reactive linker-amanitin compounds NH₂-Pro-amanitin-VA linker 35 and CN-Pro-amanitin-VA linker 37 were site-specifically conjugated to engineered cysteines. After quenching of the unconjugated toxins by N-acetyl-L-cysteine, the conjugates were purified by size-exclusion fast protein liquid chromatography (SE-FPLC) using HiLoad 16/600-Superdex 200 pg and an XK-16 column (GE Healthcare) on an ÄKTA Start System and eluted with PBS pH 7.4 at a flow rate of 1.6 mL/min. Quality analysis of the ADCs comprised size-exclusion chromatography-HPLC (SEC-HPLC) for determination of aggregates, SDS-PAGE and anti-amanitin Western Blotting under reducing and non-reducing conditions, DAR (drug-antibody ratio) analysis by liquid chromatography-mass spectrometry (LC-MS), as well as free amanitin ELISA.

### III. Results and Discussion

The retained binding affinity of selected Hyp variants on Pol II, along with the considerably reduced cytotoxic effect on OATP1B3-overexpressing HEK293 cells, prompted us to investigate ADCs based on specific variants for example, for targeted delivery to cancer cells and controlled release of the payload through cleavage of the inter-positioned protease-sensitive linker. A maleimide-based Val-Ala linker with a para-aminobenzyl conjugation site (MA-Val-Ala-PAB) was employed (Scheme 7). NH₂-Pro-amanitin (6), which showed a strong inhibitory potential on Pol II, was selected for attachment of the valine-alanine linker via carbamate linkage. Additionally, CN-Pro-amanitin (4) was modified with this Val-Ala linker through a cyclic acetal.

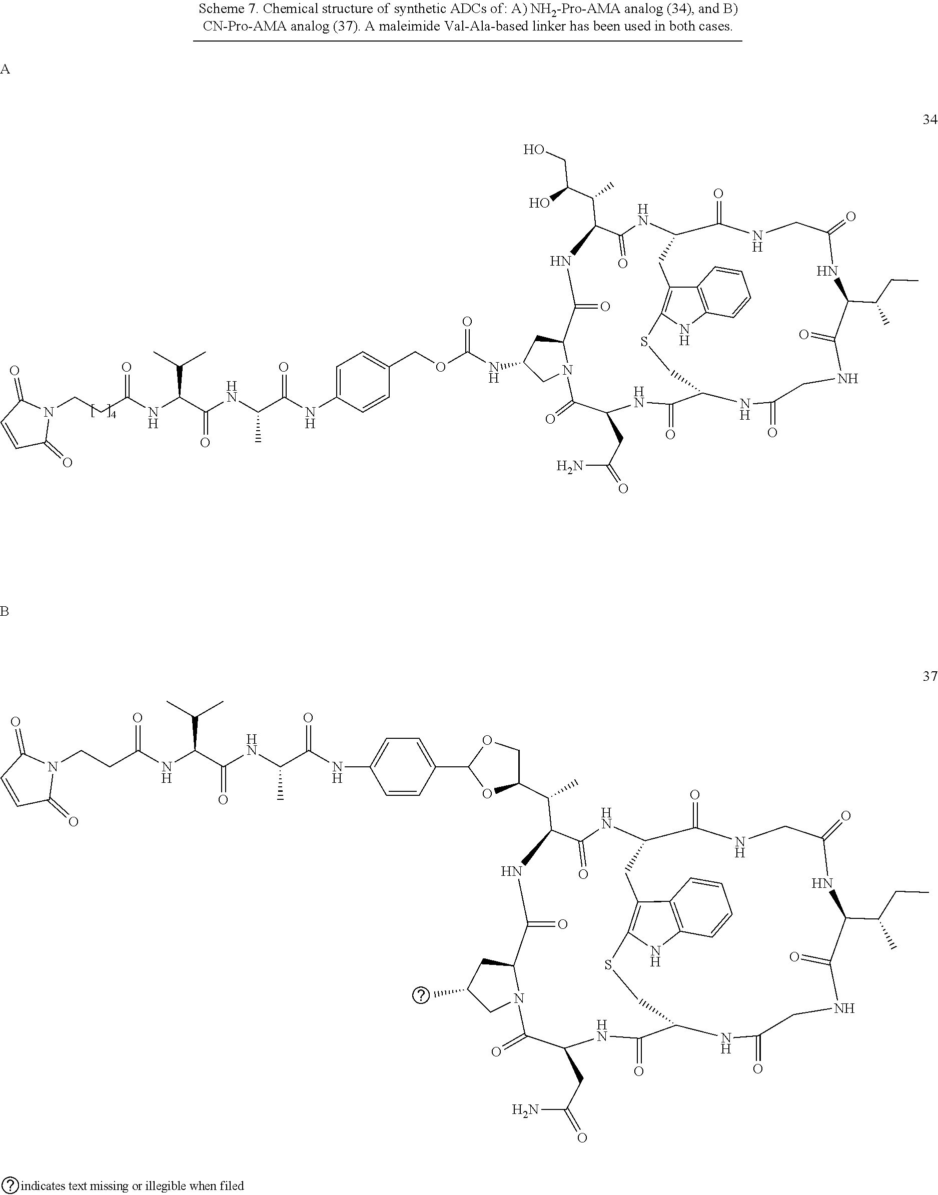

As detailed above, in the case of NH₂-Pro analog, the amine group on NH₂-Pro was conveniently used to attach the linker as a carbamate (Scheme 7, A). However, to install the linker on CN-Pro-amanitin, the two hydroxyl groups of the DHIle residue were used to form a cyclic acetal with the para-aminobenzyl terminus (Scheme 7, B). For PoC in vitro studies, these amanitin-linker variants derived from NH₂-Pro-amanitin and CN-Pro-amanitin were conjugated to the trastuzumab THIOMAB T-D265C for targeted delivery to HER2⁺ cells using site-specific conjugation chemistry to furnish the corresponding ADCs.

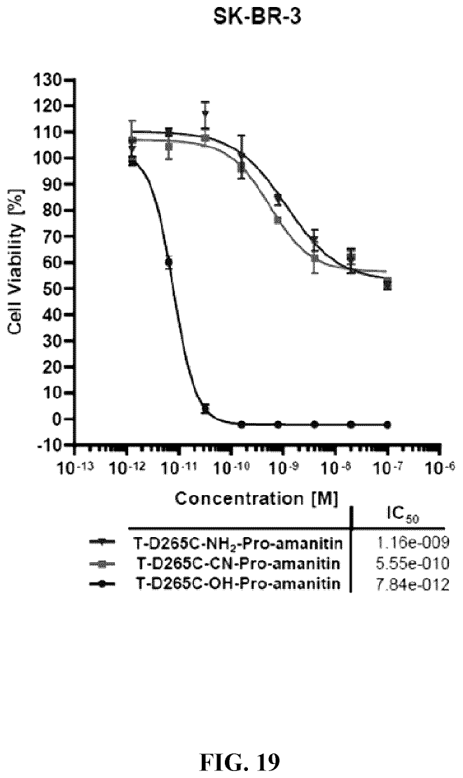

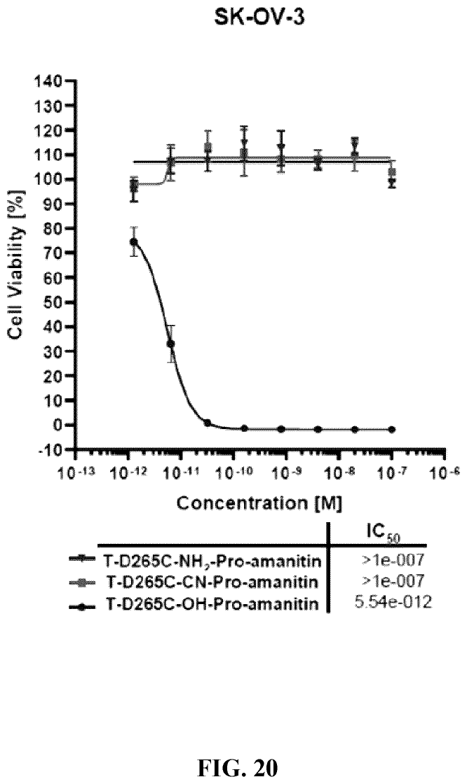

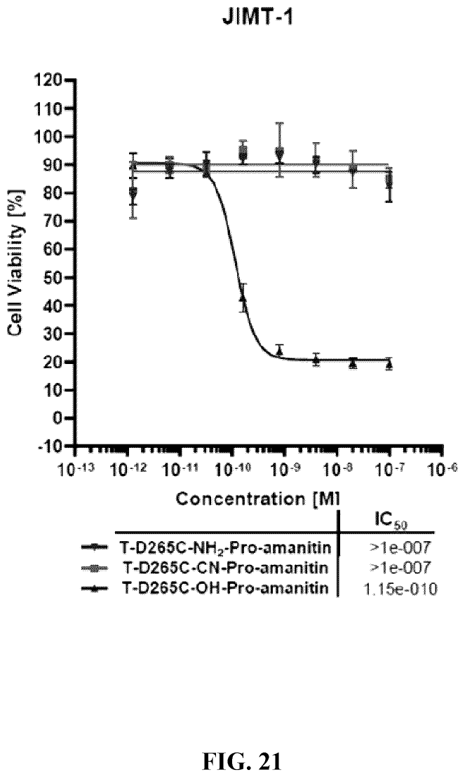

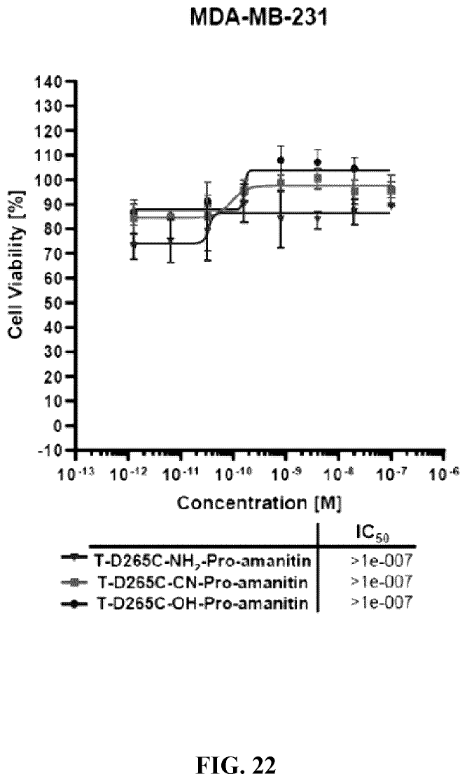

The resulting ADCs were subjected to in vitro cell-based assays against three HER2⁺ cell lines, namely SK-BR-3 (FIG. 19), SK-OV-3 (FIG. 20) and JIMT-1 (FIG. 21) and the HER2⁻ cell line MDA-MB-231 (FIG. 22). The cytotoxic potency of the two ADCs with Hyp analogs was compared to an ADC with an α-amanitin variant as the cytotoxic payload using the same Val-Ala linker (T-D265C-OH-Pro-amanitin).

All HER2⁺ cell lines showed high sensitivity towards the ADC based on α-amanitin with IC₅₀ values of 0.12±0.01 nM for JIMT-1, 7.84±0.14 pM for SK-BR-3 and 5.54±0.71 pM for SK-OV-3 cells, respectively. In contrast, JIMT-1 and SK-OV-3 cells did not show any response towards the Hyp-amanitin variants (FIG. 21 and FIG. 20, respectively). However, on SK-BR-3 cells (highest HER2-expression), the Hyp variants were able to induce a mild cytotoxic effect by reducing cell viability to approx. 50% with an IC₅₀ value of 1.16±0.89 nM for T-D265C-NH₂-Pro-amanitin, and an IC₅₀ value of 0.55±0.10 nM for T-D265C-CN-Pro-amanitin, respectively (FIG. 19). The HER2⁻ cell line MDA-MB-231 did not respond to ADC treatment indicating that no unspecific effects are responsible for the cytotoxicity on HER2⁺ cell lines (FIG. 22).

These in vitro results confirm that the Hyp residue plays a major role for the cytotoxicity and inhibitory activity of α-amanitin. However, the reduced but still substantial inhibitory activity of some of the modified amanitin derivatives show the promising potential of these analogs for therapeutic applications if used as payload for ADCs. The combination of being a poor substrate for OATP1B3 transporters whilst retaining inhibitory activity of amanitin to a certain degree might, for example help to develop ADCs with reduced payload-mediated toxicity and an improved target-specific effect. ADCs based on low potency payloads like SN38 (e.g. ENHERTU®) make use of high DAR to overcome the limitation of the payload. Additionally, the hydrophilic nature of amanitin could ease this approach.

While the invention has been described with reference to what are presently considered to be the preferred examples, it is to be understood that the invention is not limited to the disclosed examples. To the contrary, the present invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE DESCRIPTION

¹Wieland, H.; Hallermayer, R., Justus Liebigs Ann. Chem. 1941, 548, 1-18.
²Wieland, T.; Faulstich, H., CRC Critical Reviews in Biochemistry 1978, 5 (3), 185-260.
³Wieland, T., Int. J Pept. Protein Res. 1983, 22 (3), 257-276.
⁴Zanotti, G.; Mohringer, C.; Wieland, T., Int. J. Pept. Protein Res. 1987, 30 (4), 450-459.
⁵Zanotti, G.; Birr, C.; Wieland, T., Int. J Pept. Protein Res. 1981, 18 (2), 162-168.
⁶Cochetme, M.; Chambon, P., Biochim. Biophys. Acta 1974, 353 (2), 160-184.
⁷Kedinger, C.; Gniazdow. M; Mandel, J. L.; Gissinge. F; Chambon, P., Biochem. Biophys. Res. Commun. 1970, 38 (1), 165-171.
⁸Nguyen, V. T.; Giannoni, F.; Dubois, M. F.; Seo, S. J.; Vigneron, M.; Kedinger, C.; Bensaude, O., Nucleic Acids Res. 1996, 24 (15), 2924-2929.
⁹Davis, M. T. B.; Preston, J. F., Science 1981, 213 (4514), 1385-1388.
¹⁰Moldenhauer, G.; Salnikov, A. V.; Luttgau, S.; Herr, I.; Anderl, J.; Faulstich, H., J Natl. Cancer Inst. 2012, 104 (8), 622-634.
¹¹Ramkumar, P.; Abarientos, A. B.; Tian, R.; Seyler, M.; Leong, J. T.; Chen, M.; Choudhry, P.; Hechler, T.; Shah, N.; Wong, S. W.; Martin III, T. G.; Wolf, J. L.; Roybal, K. T.; Pahl, A.; Taunton, J.; Wiita, A. P.; Kampmann, M., Blood Adv 2020, 4 (13), 2899-2911.
¹² (a) Krzyscik, M. A.; Opalinski, L.; Otlewski, J., Mol. Pharm. 2019, 16 (8), 3588-3599; (b) Li, Y. J.; Liu, Y. H.; Xu, H. C.; Jiang, G. L.; Van der Jeught, K.; Fang, Y. Z.; Zhou, Z. L.; Zhang, L.; Frieden, M.; Wang, L. F.; Luo, Z. H.; Radovich, M.; Schneider, B. P.; Deng, Y. B.; Liu, Y. L.; Huang, K.; He, B.; Wang, J.; He, X. M.; Zhang, X. N.; Ji, G.; Lu, X. B., Nature Communications 2018, 9, 15; (c) Bodero, L.; Rivas, P. L.; Korsak, B.; Hechler, T.; Pahl, A.; Muller, C.; Arosio, D.; Pignataro, L.; Gennari, C.; Piarulli, U., Beilstein J. Org. Chem. 2018, 14, 407-415; (d) Zhao, L.; May, J. P.; Blanc, A.; Dietrich, D. J.; Loonchanta, A.; Matinkhoo, K.; Pryyma, A.; Perrin, D. M., ChemBioChem 2015, 16 (10), 1420-1425; (e) Moshnikova, A.; Moshnikova, V.; Andreev, O. A.; Reshetnyak, Y. K., Biochemistry 2013, 52 (7), 1171-1178.
¹³Kume, K.; Ikeda, M.; Miura, S.; Ito, K.; Sato, K. A.; Ohmori, Y.; Endo, F.; Katagiri, H.; Ishida, K.; Ito, C.; Iwaya, T.; Nishizuka, S. S., Scientific Reports 2016, 6, 15.
¹⁴Bushnell, D. A.; Cramer, P.; Kornberg, R. D., Proc. Natd. Acad. Sci. USA 2002, 99 (3), 1218-1222.
¹⁵Liu, X. Y.; Famung, L.; Wigge, C.; Cramer, P., J. Biol. Chem. 2018, 293 (19), 7189-7194.
¹⁶Brueckner, F.; Cramer, P., Nat. Struct. Mol. Biol. 2008, 15 (8), 811-818.
¹⁷Buku, A.; Altmann, R.; Wieland, T., Annalen Der Chemie-Justus Liebig 1974, (10), 1580-1586.
¹⁸Wieland, T.; Gotzendorfer, C.; Zanotti, G.; Vaisius, A. C., Eur. J. Biochem. 1981, 117 (1), 161-164.
¹⁹Shoham, G.; Rees, D. C.; Lipscomb, W. N.; Zanotti, G.; Wieland, T., J Am. Chem. Soc. 1984, 106 (16), 4606-4615.
²⁰Zanotti, G.; Petersen, G.; Wieland, T., Int. J. Pept. Protein Res. 1992, 40 (6), 551-558.
²¹Panasik, N.; Eberhardt, E. S.; Edison, A. S.; Powell, D. R.; Raines, R. T., Int. J Pept. Protein Res. 1994, 44 (3), 262-269.
²²Holmgren, S. K.; Bretscher, L. E.; Taylor, K. M.; Raines, R. T., Chem. Biol. 1999, 6 (2), 63-70.
²³DeRider, M. L.; Wilkens, S. J.; Waddell, M. J.; Bretscher, L. E.; Weinhold, F.; Raines, R. T.; Markley, J. L., J. Am. Chem. Soc. 2002, 124 (11), 2497-2505.
²⁴Matinkhoo, K.; Pryyma, A.; Todorovic, M.; Patrick, B. O.; Perrin, D. M., J Am. Chem. Soc. 2018, 140 (21), 6513-6517.
²⁵Siegert, M. A. J.; Knittel, C. H.; Sussmuth, R. D., Angew. Chem.-Int. Edit. 2020, 59 (14), 5500-5504.
²⁶Lutz, C.; Simon, W.; Werner-Simon, S.; Pahl, A.; Muller, C., Angew. Chem.-Int. Edit. 2020,59(28), 11390-11393.
²⁷Pahl, A.; Lutz, C.; Hechler, T., Cytotoxic Payloadsfor Antibody-Drug Conjugates In: Amatoxins as RNA Polymerase II Inhibiting Antibody-Drug Conjugate (ADC) Payloads CHAPTER 19, The Royal Society of Chemistry 2019, ISBN: 978-1-78801-077-1.
²⁸Wieland, T.; Gotzendorfer, C.; Dabrowski, J.; Lipscomb, W. N.; Shoham, G., Biochemistry 1983, 22 (5), 1264-1271.
²⁹Buku, A.; Wieland, T.; Bodenmuller, H.; Faulstich, H., Experientia 1980, 36 (1), 33-34.
³⁰Holmgren, S. K.; Taylor, K. M.; Bretscher, L. E.; Raines, R. T., Nature 1998, 392 (6677), 666-667.
³¹Kotch, F. W.; Guzei, I. A.; Raines, R. T., J Am. Chem. Soc. 2008, 130 (10), 2952-2953.
³²Shoulders, M. D.; Guzei, I. A.; Raines, R. T., Biopolymers 2008, 89 (5), 443-454.
³³Shoulders, M. D.; Raines, R. T., Annu. Rev. Biochem 2009, 78, 929-958.
³⁴Shoham, G.; Lipscomb, W. N.; Wieland, T., J Am. Chem. Soc. 1989,111 (13), 4791-4809.
³⁵Eggleston, D. S.; Hodgson, D. J., Int. J. Pept. Protein Res. 1985, 25 (3), 242-253.
³⁶Cadamuro, S. A.; Reichold, R.; Kusebauch, U.; Musiol, H. J.; Renner, C.; Tavan, P.; Moroder, L., Angew. Chem.-Int. Edit. 2008, 47 (11), 2143-2146.
³⁷ (a) Babu, I. R.; Ganesh, K. N., J. Am. Chem. Soc. 2001, 123 (9), 2079-2080; (b) Umashankara, M.; Babu, I R.; Ganesh, K. N., Chem. Commun. 2003, (20), 2606-2607; (c) Umashankara, M.; Nanda, M.; Sonar, M.; Ganesh, K. N., Chem. 2012, 66 (12), 936-940; (d) Nanda, M.; Ganesh, K. N., J Org. Chem. 2012, 77 (8), 4131-4135; (e) Umashankara, M.; Sonar, M. V.; Bansode, N. D.; Ganesh, K. N., J Org. Chem. 2015, 80 (17), 8552-8560; (f) Egli, J.; Siebler, C.; Maryasin, B.; Erdmann, R. S.; Bergande, C.; Ochsenfeld, C.; Wennemers, H., Chem.-Eur. J 2017, 23 (33), 7938-7944.
³⁸Kuemin, M.; Nagel, Y. A.; Schweizer, S.; Monnard, F. W.; Ochsenfeld, C.; Wennemers, H., Angew. Chem.-Int. Edit. 2010, 49 (36), 6324-6327.
³⁹Erdmann, R. S.; Wennemers, H., J. Am. Chem. Soc. 2012, 134 (41), 17117-17124.
⁴⁰ (a) Laurence, C.; Berthelot, M., Perspect. Drug Discovery Des. 2000, 18, 39-60; (b) Ziao, N.; Graton, J.; Laurence, C.; Le Questel, J. Y., Acta Crystallogr. Sect. B-Struct. Sci. Cryst. Eng. Mat. 2001, 57, 850-858; (c) Chen, D. L.; Oezguen, N.; Urvil, P.; Ferguson, C.; Dann, S. M.; Savidge, T. C., Sci. Adv. 2016, 2 (3), 16.
⁴¹ (a) Sonntag, L. S.; Schweizer, S.; Ochsenfeld, C.; Wennemers, H., J Am. Chem. Soc. 2006, 128 (45), 14697-14703; (b) Kumin, M.; Sonntag, L. S.; Wennemers, H., J Am. Chem. Soc. 2007, 129 (3), 466-467; (c) Erdmann, R. S.; Kumin, M.; Wennemers, H., Chimia 2009, 63 (4), 197-200.
⁴²Gomez-Vidal, J. A.; Forrester, M. T.; Silverman, R. B., Org. Lett. 2001, 3 (16), 2477-2479.
⁴³Pryyma, A.; Bu, Y. J.; Wai, Y.; Patrick, B. O.; Perrin, D. M., Org. Lett. 2019, 21 (20), 8234-8238.
⁴⁴Savige, W. E.; Fontana, A., Int. J. Pept. Protein Res. 1980, 15 (2), 102-112.
⁴⁵Jbara, M.; Laps, S.; Morgan, M.; Kamnesky, G.; Mann, G.; Wolberger, C.; Brik, A., Nature Communications 2018, 9, 11.
⁴⁶Cimecioglu, A. L.; Ball, D. H.; Kaplan, D. L.; Huang, S. H., Macromolecules 1994, 27 (11), 2917-2922.
⁴⁷Yagodkin, A.; Loschcke, K; Weisell, J.; Azhayev, A., Tetrahedron 2010, 66 (12), 2210-2221.
⁴⁸Camaroglio, D.; Martina, K.; Palmisano, G.; Penoni, A.; Domini, C.; Cravotto, G., Beilstein J Org. Chem. 2013, 9, 2378-2386.
⁴⁹Gao, C. Z.; Fisher, Z. B.; Edgar, K. J., Cellulose 2019, 26 (1), 445-462.
⁵⁰Tian, M. Q.; Yan, M.; Baran, P. S., J. Am. Chem. Soc. 2016, 138 (43), 14234-14237.
⁵¹Letschert, K.; Faulstich, H.; Keller, D.; Keppler, D., Toxicol. Sci. 2006, 91 (1), 140-149.
⁵²Dignam, J. D.; Lebovitz, R. M.; Roeder, R. G., NucleicAcids Res. 1983, 11 (5), 1475-1489.
⁵³Novello, F.; Fiume, L.; Stirpe, F., Biochem. J 1970, 116 (2), 177-180.
⁵⁴Lindell, T. J.; Weinberg, F.; Morris, P. W.; Roeder, R. G.; Rutter, W. J., Science 1970, 170 (3956), 447-9.
⁵⁵Krieger, F.; Moglich, A.; Kiefhaber, T., J. Am. Chem. Soc. 2005, 127 (10), 3346-3352.
⁵⁶Saha, I.; Shamala, N., Biopolymers 2013, 99 (9), 605-610.
⁵⁷Kostansek, E. C.; Lipscomb, W. N.; Yocum, R. R.; Thiessen, W. E., J. Am. Chem. Soc. 1977, 99 (4), 1273-1274.
⁵⁸Kostansek, E. C.; Lipscomb, W. N.; Yocum, R. R.; Thiessen, W. E., Biochemistry 1978, 17 (18), 3790-3795.
⁵⁹Wang, D.; Bushnell, D. A.; Huang, X. H.; Westover, K. D.; Levitt, M.; Kornberg, R. D., Science 2009, 324 (5931), 1203-1206.
⁶⁰Kaplan, C. D.; Larsson, K. M.; Komberg, R. D., Mol. Cell 2008, 30 (5), 547-556.
⁶¹Cheung, A. C. M.; Cramer, P., Cell 2012, 149 (7), 1431-1437.
⁶²Biswal, H. S.; Shirhatti, P. R.; Wategaonkar, S., J. Phys. Chem. A 2010, 114 (26), 6944-6955.
⁶³Newberry, R. W.; Raines, R. T., Topics in heterocyclic chemistry 2017, 48, 1-25.

What is claimed is:

1. A compound of formula Ama-R¹, wherein Ama is an amatoxin, and R¹ is a trans-4-substituent on the proline residue of the amatoxin, wherein R¹ is not OH.

2. The compound of claim 1, wherein Ama-R¹ is a compound of Formula I: 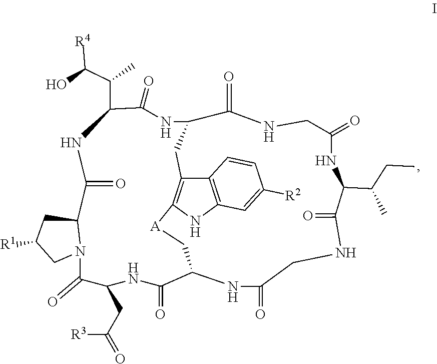
wherein
R¹ is as defined in claim 1;
R² is H or OH;
R³ is NHR⁵ or OR⁶;
R⁴ is H, CH₃, CH₂OH or CH₂OC(O)R⁷;
R⁵ is selected from H, NHR^{B}, NHOR⁹, C₁₋₆alkyl and aryl;
R⁶, R⁷, R⁸ and R⁹ are each independently selected from H, C₁₋₆alkyl and aryl; and
A is S, (R)—SO or SO₂.

3. The compound of claim 2, wherein R³ is NH₂.

4. The compound of claim 2 or 3, wherein R⁴ is CH₂OH.

5. The compound of claim 2, wherein the compound of Formula I is a compound of Formula I(a): 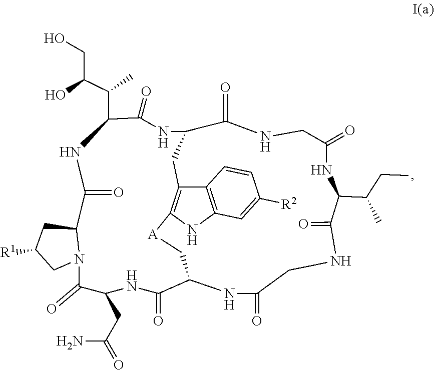
wherein
R¹ is as defined in claim 1;
R² is H or OH; and
A is S, (R)—SO or SO₂.

6. The compound of any one of claims 2 to 5, wherein R² is H.

7. The compound of any one of claims 2 to 5, wherein R² is OH.

8. The compound of any one of claims 2 to 7, wherein A is S.

9. The compound of any one of claims 2 to 7, wherein A is (R)—SO.

10. The compound of any one of claims 1 to 9, wherein R¹ is —NH₂, —NC(NH₂)₂, —CN or —SH.

11. The compound of claim 10, wherein R¹ is —CN.

12. The compound of claim 10, wherein R¹ is —NH₂.

13. The compound of claim 10, wherein R¹ is —NC(NH₂)₂.

14. The compound of claim 10, wherein R¹ is —SH.

15. A compound-linker construct comprising the compound of any one of claims 1 to 14, coupled to a linker, wherein the linker comprises a reactive group R¹⁰ for conjugating the compound-linker construct to a target-binding moiety.

16. The compound-linker construct of claim 15, wherein the linker is a stable linker.

17. The compound-linker construct of claim 15, wherein the linker is a cleavable linker.

18. The compound-linker construct of claim 17, wherein the linker further comprises a self-immolating moiety.

19. The compound-linker construct of claim 15, wherein the linker is cleavable by at least one agent selected from the group consisting of cysteine protease, metalloproteinase, serine protease, threonine protease and aspartic protease.

20. The compound-linker construct of any one of claims 17 to 19, wherein the linker comprises a motif selected from the group consisting of Val-Ala, Val-Cit, Val-Lys, Val-Arg, Phe-Lys-Gly-Pro-Leu-Gly, Ala-Ala-Pro-Val, β-glucuronide and β-galactoside.

21. The compound-linker construct of any one of claims 15 to 20, wherein R¹⁰ is selected from: 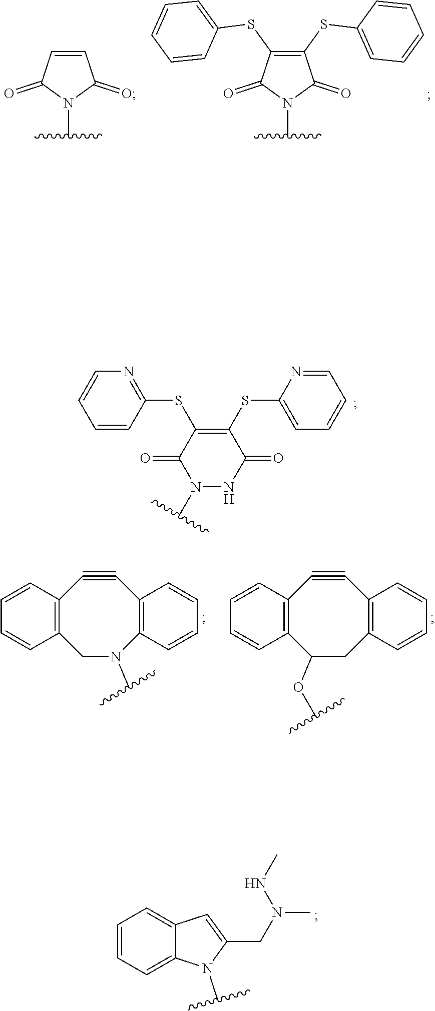 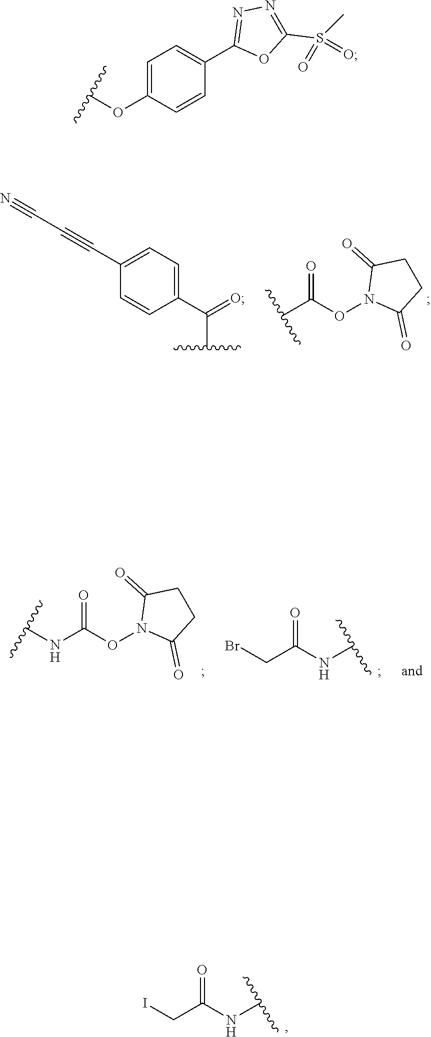
wherein [symbol] represents the site of attachment of R¹⁰ to the remainder of the linker.

22. The compound-linker construct of claim 21, wherein R¹⁰ is

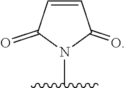 [SMILES: *N1C(=O)C=CC1=O]

23. The compound-linker construct of claim 15, wherein the linker-R¹⁰ comprises a motif of the following structure: 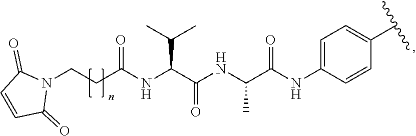
wherein n is an integer from 1 to 6 and [symbol] represents the site of coupling of the linker to the compound or to a functional group that couples the linker to the compound.

24. The compound-linker construct of any one of claims 15 to 23, as dependent from any one of claims 1 to 10, wherein Ama comprises a 4,5-dihydroxyleucine moiety and the linker is coupled to the compound via a cyclic acetal obtained from reaction of the hydroxyl groups of the 4,5-dihydroxyisoleucine moiety with a ketone moiety on the linker.

25. The compound-linker construct of claim 24, wherein R¹ is —CN.

26. The compound-linker construct of any one of claims 23 to 25, wherein n is 1.

27. The compound-linker construct of any one of claims 15 to 23, as dependent from any one of claims 1 to 10, wherein the linker is coupled to the compound via reaction of R¹ with a reactive group thereto on the linker.

28. The compound-linker construct of claim 27, wherein R¹ is —NH₂.

29. The compound-linker construct of claim 28, wherein the reactive group is a para-nitrophenyl ester.

30. The compound-linker construct of any one of claims 15 and 27 to 29, wherein n is 4.

31. A conjugate comprising a target-binding moiety conjugated to a compound of any one of claims 1 to 14, or a compound-linker construct of any one of claims 15 to 30.

32. The conjugate of claim 31, wherein the conjugate comprises the compound-linker construct, R¹⁰ is a thiol-reactive group, the target-binding moiety comprises an engineered cysteine residue and the compound-linker construct is conjugated to the target-binding moiety via a moiety resulting from the reaction of the thiol of the engineered cysteine residue with R¹⁰.

33. The conjugate of claim 32, wherein the engineered cysteine residue is selected from the group consisting of heavy chain 118Cys, heavy chain 239Cys, and heavy chain 265Cys.

34. The conjugate of claim 33, wherein the engineered cysteine residue is heavy chain 265Cys.

35. The conjugate of any one of claims 31 to 34, wherein the target-binding moiety is an antibody, an antigen-binding fragment thereof or an antibody-like protein.

36. The conjugate of claim 35, wherein the target-binding moiety is an anti-HER2 antibody.

37. A pharmaceutical composition comprising the compound of any one of claims 1 to 14, or the conjugate of any one of claims 31 to 36 and a pharmaceutically acceptable carrier.

38. A use of an effective amount of a compound of any one of claims 1 to 14, or a conjugate of any one of claims 31 to 36 for treatment of a disease associated with cells presenting a target in a subject in need thereof, wherein the target-binding moiety is specific for the target.

39. A use of an effective amount of a compound of any one of claims 1 to 14, or a conjugate of any one of claims 31 to 36 for preparation of a medicament for treatment of a disease associated with cells presenting a target in a subject in need thereof, wherein the target-binding moiety is specific for the target.

40. The use of claim 38 or 39, wherein the target-binding moiety is an anti-HER2 antibody and the disease is HER2-positive breast cancer.

41. A use of an effective amount of a compound of any one of claims 1 to 14, or a conjugate of any one of claims 31 to 36 for treatment of cancer in a subject in need thereof.

42. A use of an effective amount of a compound of any one of claims 1 to 14, or a conjugate of any one of claims 31 to 36 for preparation of a medicament for treatment of cancer in a subject in need thereof.

43. A compound of any one of claims 1 to 14, or a conjugate of any one of claims 31 to 36 for use to treat a disease associated with cells presenting a target in a subject, wherein the target-binding moiety is specific for the target.

44. The compound or conjugate for the use of claim 43, wherein the target-binding moiety is an anti-HER2 antibody and the disease is HER2-positive breast cancer.

45. A compound of any one of claims 1 to 14, or a conjugate of any one of claims 31 to 36 for use to treat cancer in a subject.

46. A method of treating a disease associated with cells presenting a target in a subject in need thereof, the method comprising administering an effective amount of a compound of any one of claims 1 to 14, or a conjugate of any one of claims 31 to 36 to the subject, wherein the target-binding moiety is specific for the target.

47. The method of claim 46, wherein the target-binding moiety is an anti-HER2 antibody and the disease is HER2-positive breast cancer.

48. A method of treating cancer in a subject in need thereof, the method comprising administering an effective amount of a compound of any one of claims 1 to 14, or a conjugate of any one of claims 31 to 36 to the subject.
